(19) United States
(12) Patent Application Publication
(10) Pub. No.: US 2022/0125951 A1
(43) Pub. Date: Apr. 28, 2022

(54) MICROENVIRONMENT SENSORS TO REGULATE ENGINEERED GENE EXPRESSION

(71) Applicant: Seattle Children's Hospital (dba Seattle Children's Research Institute), Seattle, WA (US)

(72) Inventors: Courtney Crane, Seattle, WA (US); Jennifer Gardell, Seattle, WA (US); Harrison Kikuo Chinn, Seattle, WA (US)

(73) Assignee: Seattle Children's Hospital (dba Seattle Children's Research Institute), Seattle, WA (US)

(21) Appl. No.: 17/424,140

(22) PCT Filed: Jan. 30, 2020
(86) PCT No.: PCT/US2020/015809

§ 371 Date: Jul. 19, 2021

Related U.S. Application Data
(60) Provisional application No. 62/800,049, filed on Feb. 1, 2019.

(51) Int. Cl. A61K 48/00; C12N 15/86

(52) U.S. Cl. CPC A61K 48/0066; C12N 2740/15043; C12N 15/86

(57) ABSTRACT

Some embodiments of the methods and compositions provided herein relate to transgenes comprising regulatory elements capable of inducing specific transcription of an operably-linked therapeutic payload in a cell in an in vivo microenvironment. In some embodiments, the regulatory elements are responsive to endogenous stimuli of the microenvironment. In some embodiments, the regulatory elements are response to stimuli from chimeric receptors in the cell.

### CROSS-REFERENCE TO RELATED APPLICATIONS

This application claims priority to U.S. Prov. App. No. 62/800,049 filed Feb. 1, 2019 entitled “MICROENVIRONMENT SENSORS TO REGULATE ENGINEERED GENE EXPRESSION,” which is hereby expressly incorporated by reference in its entirety.

### REFERENCE TO SEQUENCE LISTING

The present application is being filed along with a Sequence Listing in electronic format. The Sequence Listing is provided as a file entitled SCRI207WOSEQLIST, created Jan. 21, 2020, which is approximately 105 Kb in size. The information in the electronic format of the Sequence Listing is incorporated herein by reference in its entirety.

### FIELD OF THE INVENTION

Some embodiments of the methods and compositions provided herein relate to transgenes comprising regulatory elements configured to induce transcription of an operably-linked therapeutic payload in a cell in an in vivo microenvironment. In some embodiments, the regulatory elements are responsive to endogenous stimuli presented by the microenvironment. In some embodiments, the regulatory elements are responsive to stimuli from a chimeric receptor on a cell.

### BACKGROUND OF THE INVENTION

Modulation of a patient's immune system using immunotherapeutic approaches has shown remarkable success against hematological neoplasms and some solid tumors, including metastatic melanoma and colorectal carcinoma. In contrast to these successes, solid tumors, including glioblastoma (GBM) tumors have not yet responded to immunotherapy approaches. This is largely due to the fact that many solid tumors and the microenvironments that they create are highly immunosuppressive and tumor promoting, supporting tumor growth and preventing the localization and functions of cytotoxic immune cells. Therefore, an approach to overcome the influence of the tumor microenvironment (TME) and the impact on infiltrating immune cells that are responsible for the elimination of transformed cells is required as a first step in developing successful immunotherapies for GBM and other solid tumors.

For example, while childhood leukemias have shown remarkable responses to T cell-based therapeutics; treatment of solid tumors has not been nearly as successful. Along with a lack of tumor-specific antigens, the immunosuppressive microenvironment of many solid tumors has thus far been an insurmountable barrier, precluding CAR T-cell immunotherapy. Solid tumors, such as brain tumors, which represent 20% of childhood cancers, are highly infiltrated by myeloid cells that render the tumor highly resistant to the cytotoxic functions. As such, an approach to overcome the influence of the TME and the impact on infiltrating immune cells that are responsible for the elimination of transformed cells is strongly needed as a first step in developing successful immunotherapies for GBM and other solid tumors.

### SUMMARY OF THE INVENTION

Some embodiments of the methods and compositions provided herein include a polynucleotide comprising: a first nucleic acid comprising a regulatory element, wherein the regulatory element is capable of or is configured to induce transcription of a therapeutic payload in a cell in an in vivo microenvironment; and a second nucleic acid encoding the payload, wherein the therapeutic payload is operably-linked to the first nucleic acid.

In some embodiments, the in vivo microenvironment is selected from a tumor microenvironment, or an inflammation microenvironment.

In some embodiments, specific transcription is induced by the regulatory element in response to a stimulus in the microenvironment. In some embodiments, the stimulus comprises: an increased level of a protein or nucleic acid encoding the protein, in the microenvironment as compared to a systemic circulation selected from vascular endothelial growth factor (VEGF), transforming growth factor (TGF), a tumor necrosis factor (TNF), IL-6, an interferon, C3b, or macrophages colony-stimulating factor (M-CSF); or decreased levels of oxygen in the microenvironment, as compared to a systemic circulation.

In some embodiments, specific transcription is induced by the regulatory element in response to a stimulus from a chimeric receptor in the cell. In some embodiments, the stimulus comprises a phosphorylated Syk protein.

In some embodiments, the regulatory element comprises a promoter, an enhancer, or a functional fragment thereof capable of or configured to induce specific transcription of a payload in a cell in a tumor microenvironment.

In some embodiments, the promoter, enhancer, or functional fragment thereof is derived from or selected from APOE, C1QA, SPP1, RGS1, C3, HSPA1B, TREM2, A2M, DNAJB1, HSPB1, NR4A1, CCL4L2, SLC1A3, PLD4, HSPA1A, OLR1, BIN1, CCL4, GPR34, EGR1, HLA-DQA1, FCGR3A, VSIG4, LILRB4, CSF1R, HSPA6, TUBA1B, BHLHE41, GSN, JUN, CX3CR1, HLA-DQB1, HSPE1, FCGR1A, CCL3L1, OLFML3, ADAM28, YWHAH, GADD45B, SLCO2B1, HSP90AA1, HSPA8, RNASET2, HLA-DPA1, CDKN1A, CD83, HAVCR2, DDIT4, C3AR1, HSPD1, LGMN, TMIGD3, CD69, IFI44L, SERPINEL HLA-DMA, ALOX5AP, EPB41L2, HSP90AB1, HSPH1, RHOB, CH25H, FRMD4A, CXCL16, FCGR1B, HLA-DMB, GPR183, HLA-DPB1, SLC2A5, EGR2, ID2, RGS10, APBB HP, EVL, CSF2RA, SGK1, FSCN1, BEST1, ADORA3, IFNGR1, MARCKS, MT2A, SRGAP2, ARL5A, ADGRG1, HMOX1, RHBDF2, ATF3, SOCS6, NR4A3, PLK3, APMAP, AKR1B1, UBB, HERPUD1, CTSL, BTG2, IER5, LPAR6, USP53, ST6GAL1, ADAP2, HTRA1, KCNMB1, DNAJAL LPCAT2, ZFP36L1, CCL3, BAG3, TMEM119, LTC4S, EGR3, FCGBP, ABI3, IFNγ, TNFα, IFNα, IL-6, or IL-12.

In some embodiments, the regulatory element comprises an element selected from a hypoxia response element (HRE), a SRC binding element, a SMAD 2 response element, a SMAD 3 response element, an ATF binding site, a STAT 2 binding site, a CBP binding site, or a SYK binding element. In some embodiments, the regulatory element comprises an HRE.

In some embodiments, the therapeutic payload encodes a cytokine.

In some embodiments, the therapeutic payload encodes an interferon. In some embodiments, the interferon is selected from interferon alpha, interferon beta, or interferon gamma.

In some embodiments, the therapeutic payload encodes a tumor necrosis factor (TNF). In some embodiments, the TNF is selected from TNF-alpha, TNF-beta, TNF-gamma, CD252, CD154, CD178, CD70, CD153, or 4-1BBL.

In some embodiments, the therapeutic payload encodes an interleukin. In some embodiments, the interleukin is selected from IL-10 IL-12, IL-1, IL-6, IL-7, IL-15, IL-2, IL-18 or IL-21.

In some embodiments, the therapeutic payload encodes a chemokine. In some embodiments, the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15.

In some embodiments, the regulatory element further comprises a constitutive promoter. In some embodiments, the constitutive promoter is selected from a MiniTK promoter, or an EFla promoter

Some embodiments also include a third nucleic acid comprising a vector. In some embodiments, the vector comprises a viral vector. In some embodiments, the vector comprises a lentiviral vector.

Some embodiments of the methods and compositions provided herein include a cell comprising any one of the foregoing polynucleotides. Some embodiments also include a polynucleotide encoding a chimeric receptor, wherein the chimeric receptor comprises an extracellular binding domain, a transmembrane domain, and an intracellular signaling domain.

In some embodiments, the extracellular binding domain, the transmembrane domain, or the intracellular signaling domain is derived from a receptor selected from a LILRB receptor, CD115 receptor, M-CSF receptor; CXCR4; Neuropilin (NRP2); Epidermal Growth Factor receptor; Vascular Endothelial Growth Factor receptor 2; Transforming Growth Factor beta receptor 2; Tumor necrosis factor alpha receptor; Interleukin 6 receptor; Interferon gamma receptor 2; Granulocyte-macrophages colony-stimulating factor receptor subunit alpha; Toll Like receptor 4; Cytokine receptors; TGFb; GM-CSF; IL-6; IL-4; IL-1beta; IL-13; IL-10; IFN-alpha, beta, gamma; Chemokine receptors; CCR1-10; CXCR1, 2, 3, 4, 5, 6; Growth Factor receptor; PDGF; VEGF; EGF; LPS receptor; LDH receptor; MDH receptor; CpG receptor; ssRNA receptor; or Folate receptor. In some embodiments, the extracellular domain is derived from an extracellular domain of a protein selected from LILRB, or CD115.

In some embodiments, the transmembrane domain is derived from a transmembrane domain of a protein selected from an IgG4 hinge connected to a CH2 domain to a CH3 domain, an IgG4 hinge connected to a CH3 domain, or an IgG4 hinge domain.

In some embodiments, the intracellular signaling domain is derived from an intracellular domain of a protein selected from CD3, or 41BB.

In some embodiments, the cell is an immune cell.

In some embodiments, the cell is a myeloid cell. In some embodiments, the cell is selected from a basophil, neutrophil, esosinophil, or monocyte. In some embodiments, the cell is a macrophage. In some embodiments, the cell is prepared by contacting a monocyte with GM-CSF and/or M-CSF to obtain a macrophage.

In some embodiments, the cell is a lymphoid cell. In some embodiments, the cell is selected from a natural killer cell, or a T cell.

In some embodiments, the cell is mammalian. In some embodiments, the cell is human.

In some embodiments, the cell is an ex vivo cell.

Some embodiments of the methods and compositions provided herein include a method of treating, inhibiting or ameliorating a disorder in a subject, comprising: administering any one of the foregoing cells to the subject. Accordingly, use of any one or more of the aforementioned compositions as a medicament are contemplated.

In some embodiments, the disorder is selected from a cancer, or an inflammatory disorder. Accordingly, any one or more of the compositions described herein for treating a cancer or an inflammatory disease are also contemplated.

In some embodiments, the disorder is a cancer. In some embodiments, the cancer comprises a solid tumor. In some embodiments, the cancer is selected from a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcoma, neuroblastoma, prostate cancer, or ovarian cancer. In some embodiments, the cancer is a glioblastoma.

In some embodiments, the disorder is an inflammatory disorder or inflammatory disease. In some embodiments, the inflammatory disorder or inflammatory disease is selected from acne vulgaris, asthma, certain autoimmune diseases, certain autoinflammatory diseases, celiac disease, chronic prostatitis, colitis, diverticulitis, glomerulonephritis, hidradenitis suppurativa, certain hypersensitivities, certain inflammatory bowel diseases, interstitial cystitis, lichen planus, mast cell activation syndrome, mastocytosis, otitis, pelvic inflammatory disease, reperfusion injury, rheumatic fever, rheumatoid arthritis, rhinitis, sarcoidosis, transplant rejection, vasculitis, acute bacterial infection, chronic bacterial infection, post-transplant associated inflammation, or post-transplant associated inflammation suppression.

In some embodiments, the subject is mammalian. In some embodiments, the subject is human.

### BRIEF DESCRIPTION OF THE DRAWINGS

FIG. 1 depicts constructs including: (A) a CD19t construct encoding a truncated CD19 (CD19t); (B) an EF1 construct including an eF 1a promoter and a GFP/luciferase reporter gene; (C) a miniTK construct including a minimal thymidine kinase promoter and a GFP/luciferase reporter gene; and (D) an HRE miniTK construct including a series of three hypoxia response elements (EIRE), a minimal thymidine kinase promoter and a GFP/luciferase reporter gene (HRE MiniTK eGFP:ffluc-t2a-CD19t).

FIG. 2 depicts a graph of the level of luminescence in 293T cells or Raji cells transduced with a transgene comprising hypoxia response elements and a luciferase reporter gene, incubated for 20 hr in a hypoxia chamber; control transduced cells were incubated at normal levels of oxygen (normoxia).

FIG. 3 depicts a graph of levels of variability for the level of luminescence in 293T cells or Raji cells transduced with a transgene and incubated for 20 hr in a hypoxia chamber; control transduced cells were incubated at normal levels of oxygen (normoxia).

FIG. 4 depicts a graph of the levels of luminescence in primary human macrophages transduced with various transgenes and either incubated for 24 hr in a hypoxia chamber; control transduced cells were incubated at normal levels of oxygen (normoxia).

FIG. 5 depicts a graph of the relative levels of luminescence in primary human macrophages transduced with various transgenes and either incubated for 24 hr in a hypoxia chamber; control transduced cells were incubated at normal levels of oxygen (normoxia).

FIG. 6 depicts a Western blot prepared from protein extracts from primary human macrophages transduced with various transgenes before incubation in a hypoxia chamber.

FIG. 7 depicts a graph of the relative levels of luciferase protein expression in primary human macrophages transduced with various transgenes before incubation in a hypoxia chamber.

FIG. 8 depicts a graph of the relative levels of luminescence in 293T cells after removal of the cells from a hypoxia chamber.

FIG. 9A depicts a graph of the relative levels of luciferase gene expression in primary human macrophages transduced with various transgenes up to 2 days after removal of the cells from a hypoxia chamber.

FIG. 9B depicts a graph of the relative levels of luciferase gene expression in primary human macrophages transduced with various transgenes up to 5 days after removal of the cells from a hypoxia chamber.

FIG. 10 depicts a graph of the relative levels of luciferase gene expression in primary human macrophages transduced with various transgenes up to 5 days after removal of the cells from a hypoxia chamber. For each time point, 1st 2^{nd}, and 3rd columns are fold change for cells transduced with an EFla construct, a miniTK construct, or an HRE-miniTK construct, respectively.

FIG. 11 depicts a graph of the relative levels of luciferase protein expression in primary human macrophages transduced with various transgenes up to 3 days after removal of the cells from a hypoxia chamber.

FIG. 12 depicts a graph of the relative levels of luciferase protein expression in primary human macrophages transduced with various transgenes up to 5 days after removal of the cells from a hypoxia chamber. For each time point, 1^{st} and 2^{nd} columns are relative luciferase expression in cells transduced with a miniTK construct, or an HRE-miniTK construct, respectively.

FIG. 13A depicts a schematic of systemic injection of a subject at day 0 with 1×10⁶ U87 cells, and systemic injection of the subject at day 11 with 1×10⁶ genetically engineered macrophages (GEMs) containing a test transgene comprising hypoxia response elements and a luciferase reporter gene or a control transgene (left panel). Right panel depicts detection of luminescence in subjects receiving the therapy at day 1, day 6 and day 8 for subjects that had been administered the test transgene or a control transgene.

FIG. 13B depicts a graph for average radiance from GEMs transduced with a construct containing a HRE MiniTK eGFP:ffluc-t2a-CD19t, or a construct containing a CD19t.

FIG. 13C depicts photographs showing levels and location of luciferase expression in mice containing U87 glioblastoma tumors and injected with GEMs containing a CD19t construct (left panel), or a HRE MiniTK eGFP:ffluc-t2a-CD19t construct (right panel).

FIG. 13D is a series of photographs showing levels and location of luciferase expression in mice containing flank U87 glioblastoma tumors and injected with GEMs containing a HRE MiniTK eGFP:ffluc-t2a-CD19t construct.

FIG. 13E is a series of photographs showing levels and location of luciferase expression in mice containing intracranial U87 glioblastoma tumors and injected with PBS, or GEMs containing a HRE MiniTK eGFP:ffluc-t2a-CD19t construct at doses of 2.5e6 cells, or 5e6 cells.

FIG. 14 depicts a graph of the in vitro concentration of IL-12 in supernatant from primary human macrophages transduced with various transgenes up to 5 days after removal of the cells from a hypoxia chamber.

FIG. 15A depicts constructs including: (A) an EFla construct including an eF 1a promoter (EF1a), and encoding a truncated CD19 (CD19t), and human interleukin 12 p40 and p35 subunits (hIL21p40p35); (B) a miniTK construct including a minimal thymidine kinase promoter (miniTK) and encoding a CD19t, and hIL21p40p35; (C) an HRE miniTK construct including a series of three hypoxia response elements (HRE), a miniTK promoter and encoding a CD19t, and hIL21p40p35; (D) an EFla GFP-luciferase construct including an EF1a promoter, and encoding a GFP/luciferase reporter (eGFP:ffluc), and hIL21p40p35; (E) an miniTK GFP-luciferase construct including a miniTK promoter, and encoding eGFP:ffluc and hIL21p40p35; and (F) an HRE miniTK GFP-luciferase construct including a series of three HREs, a miniTK promoter and encoding eGFP:ffluc and hIL21p40p35.

FIG. 15B depicts a graph of the in vitro concentration of IL-12 in supernatant from primary human macrophages transduced with lentiviral vectors containing constructs A, B, or C, over a period of 21 days. For each time point, the 1^{st}, 2^{nd}, and 3^{rd} columns are IL-21 levels for cells transduced with constructs A, B, or C, respectively.

FIG. 15C depicts a flow cytometry study in which transduced cells were treated with either hypoxic or normoxic conditions, and sorted according to GFP expression. In FIG. 15C, left upper and lower panels represent sorted cells transduced with a positive control EF1a construct; center upper and lower panels represent sorted cells transduced with a negative control miniTK construct; and right upper and lower panels represent sorted cells transduced with an HRE-miniTK construct.

FIG. 16 depicts a graph of relative levels of GFP expression with regard to percentage GFP+ EPCAM+ cells in colorectal carcinoma slices cultured with GEMs in hypoxic conditions in which the GEMs contain an EFL1a construct, a miniTK construct, or an HRE-miniTK construct.

FIG. 17A is a schematic of an embodiment of a system in which tumor cells express M-CSF, which binds to a chimeric receptor expressed on the surface of a macrophage, the chimeric receptor comprising a CD115 domain, a transmembrane linker, and a TLR cytoplasmic domain. Binding of M-CSF to the CD115 domain induces intracellular signaling from the TLR4 domain, which activates endogenous gene expression from genes such as IL-12, IL-1, IL6, TNF, or ROS.

FIG. 17B is a schematic of an embodiment of a system in which tumor cells express MHCI, which binds to a chimeric receptor expressed on the surface of a macrophage, the chimeric receptor comprising a LILRB domain, a transmembrane linker, and a CD3ξ/41BB cytoplasmic domain. Binding of MHC I molecules to the LILRB domain induces intracellular signaling from the CD3ξ/41BB domain, which induces phosphorylation of SYK protein, which in turn activates gene expression from transgenes containing a lentiviral vector backbone (epHIV7.2), a phosphorylated SYK binding element (pSyk), and a payload, such as IL-12.

FIG. 18A depicts a map of a vector containing an example polynucleotide for the chimeric receptor.

FIG. 18B depicts a map of a vector containing an example polynucleotide for a transgene comprising regulatory elements response to phosphorylated Syk.

FIG. 18C depicts a micrograph of genetically engineered primary human macrophages (GEMs) containing a control CD19t transgene (left panel), or a test transgene encoding a LILRB1 chimeric receptor (right panel) and stained for phosphorylated syk (arrows).

FIG. 18D depicts a micrograph of genetically engineered primary human macrophages (GEMs) containing a control CD19t transgene (left panel), or a test transgene encoding a LILRB1 chimeric receptor (right panel) and stained for autologous CFSE labeled T cells (center of crosshairs).

FIG. 19A depicts an embodiment of a chimeric receptor containing a MCSF receptor extracellular domain (MCSF-R ECD), a hinge domain, a CD28 transmembrane domain (CD28TM), a TLR4 intracellular domain (TLR4.ISD), a T2A ribosome skip sequences, and a truncated CD19 marker domain (CD19t).

FIG. 19B depicts graphs of the in vitro levels of TNF-alpha or IL-12 from cells stimulated with M-CSF or LPS/IFN-gamma, and containing chimeric receptors (CR-1, or CR-2), or cells containing no chimeric receptor (UT).

FIG. 20A depicts an embodiment of a chimeric receptor containing a MCSF receptor extracellular domain which also included a hinge domain, a CD28 transmembrane domain, a TLR4 intracellular domain (MCSFR.TLR4), and also a reporter luciferase gene, a T2A ribosome skip sequences, and a truncated CD19 marker domain (CD19t).

FIG. 20B depicts photographs of xenograft mouse models administered U87 cells, and genetically modified macrophages containing either the chimeric receptor of FIG. 23A, or a CD19t control.

FIG. 21 depicts an example protocol for determining differential gene expression.

FIG. 22A depicts the number of mRNAs mapping to known translated sequences in the human genome that are detected per cell following 10× genomics single cell mRNA sequencing using two different single cell analysis algorithms, nGene and nUMI. Each dot represents a cell from a representative analysis of monocytes

FIG. 22B depicts the fraction of immune cell types contained in scRNAseq samples following 10× Genomics single cell capture and library preparation, as defined by known gene signatures for each cell type of cells prior to (left) and after (right) magnetic selection for myeloid cells. Following CD14 selection, the percentage of monocytes and macrophages significantly increases.

FIG. 23 depicts a nanostring heat map expression analysis of a myeloid panel of 770 genes. Lane 1: low grade; lane 2: GBM; lane 3: monocytes low grade glioma patient; lane 4: monocytes GBM patient; lane 5: in vitro cultured GM-CSF macrophages; lane 6: in vitro cultured M-CSF macrophages.

FIG. 24A depicts a graph for relative level of expression for certain genes in glioma patients over survival time.

FIG. 24B depicts a graph for relative level of expression for certain genes in ovarian cancer patients over time to relapse.

FIG. 24C depicts a graph for relative level of expression for certain genes in ovarian cancer patients over survival time.

FIG. 25 depict a graph for a principal component analysis of patient monocytes and matched tumor associated macrophages (TAMs).

FIGS. 26A-26N depict graphs for relative levels of certain gene expression for circulating monocytes (mono) and TAMs for genes: C1QA, C1QB, C1QC, C3, CSF1R, CCL2, RGS1, DNAJB1, HSPA6, SPP1, TREM2, TUBA1B, DNASE2, and APOE, respectively.

### DETAILED DESCRIPTION

Some embodiments of the methods and compositions provided herein relate to transgenes comprising regulatory elements capable of or configured to induce specific transcription of an operably-linked therapeutic payload in a cell in an in vivo microenvironment. In some embodiments, the regulatory elements are responsive to endogenous stimuli presented by the microenvironment. In some embodiments, the regulatory elements are a response to stimuli from chimeric receptors on the cell. In some embodiments, a microenvironment includes a tumor microenvironment (TME), and/or an inflammatory microenvironment.

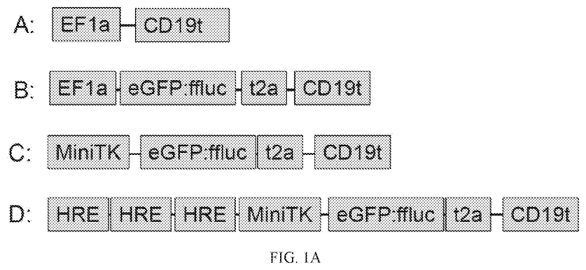

Some embodiments include polynucleotides, and/or cells containing such polynucleotides in which the polynucleotide includes or comprises a regulatory element capable of or configured to induce specific transcription of an operably-linked therapeutic payload. In some such embodiments, the regulatory elements induce specific transcription in response to a stimulus. In some embodiments, the stimulus comprises a signal associated with a microenvironment. In some embodiments, the signal is associated with a microenvironment, and the signal can include or comprise an increased or decreased level of certain signaling molecules compared to levels in other compartments of an organism, such as other populations of cells and/or tissues. In some embodiments, the signal can include or comprise a decreased level of oxygen, such as an hypoxic condition presented in a microenvironment, compared to levels of oxygen in other compartments of an organism, such as in the vicinity of other populations of cells and/or tissues. In some such embodiments, the cell is a macrophage. Example polynucleotides are depicted in FIG. 1, including construct D.

In some embodiments, the stimulus is provided by an activated chimeric receptor in a cell containing the polynucleotide. In some such embodiments, the chimeric receptor is activated by signals from a microenvironment, such as an increased or decreased level of certain signaling molecules as compared to levels in other compartments of an organism, such as other populations of cells and/or tissues; and/or the presence of certain activated immune cells. An exemplary chimeric receptor and inducible polynucleotide in a cell are depicted in FIG. 1C. In some such embodiments, the cell is a macrophage.

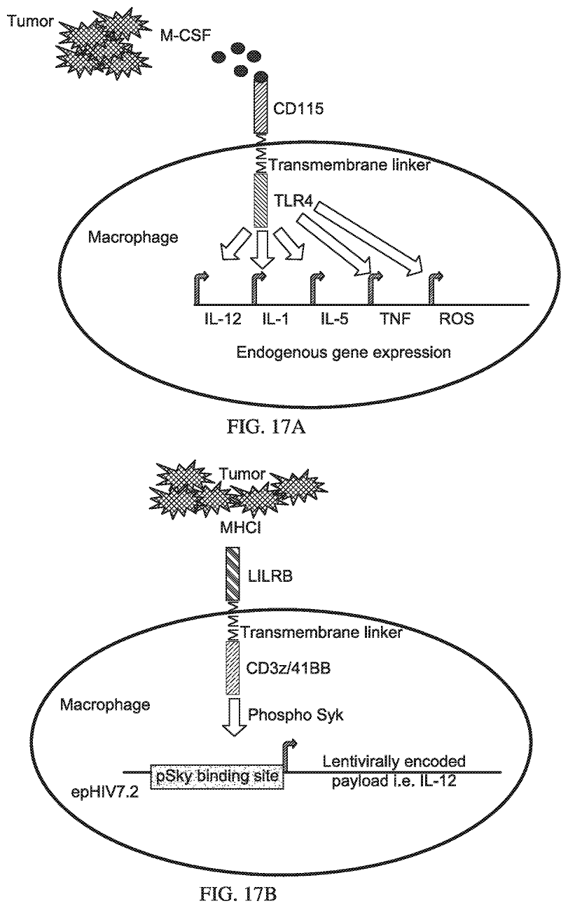

In some embodiments, a cell can contain a chimeric receptor. In some such embodiments, the chimeric receptor in a cell is activated and thereby induces specific transcription of genes endogenous to the cell. In some such embodiments, the chimeric receptor is activated by signals presented in a microenvironment, such as an increased or decreased level of certain signaling molecules as compared to levels in other compartments of an organism, such as other populations of cells and/or tissues; and/or the presence of certain activated immune cells. An example chimeric receptor in a cell is depicted in FIG. 17A. In some such embodiments, the cell is a macrophage.

Certain methods and compositions disclosed in U.S. 2017/0087185, which is expressly incorporated by reference herein in its entirety, are useful with the methods and compositions provided herein.

Some embodiments provided herein relate to immune cell therapy of subjects having inaccessible, multifocal, and/or metastatic disease. In some such embodiments, a lentiviral vector encoding a therapeutic gene is administered systemically, and expression from the vector is specific to a microenvironment in the subject, such as a TME.

Accordingly, a cohort of subjects that may have been previously ineligible for certain cellular therapies may be eligible for such therapies in combination with some embodiments of the methods and combinations provided herein. For example, some potential subjects for a chimeric antigen receptor (CAR) T cell therapy may express target antigens in both healthy tissues and targeted tumor tissues. Administration of the CAR T cell therapy to such potential subjects may cause adverse side-effects. In some embodiments, a CAR T cell therapy can be combined with certain methods and compositions provided herein and targeted to a microenvironment, such as a TME.

Some embodiments provided herein include TME sensing promoter constructs and TME inducible chimeric receptors that activate gene expression in vitro and in vivo in response to microenvironmental stimuli that are restricted to tumor tissues. Following removal from conditions that mimic the TME in vitro, lentiviral gene expression demonstrated an off rate of 2-5 days, demonstrating that as tumor burden is reduced, lentivirally encoded therapeutic payloads were no longer expressed.

In some embodiments, TME sensing promoter constructs and/or TME inducible chimeric receptors, manipulate a TME by regulating gene expression and/or improving immune cell trafficking to a tumor. In some embodiments, TME sensing promoter constructs and/or TME inducible chimeric receptors define parameters or regions in the TME, such as areas of rapid tumor cell proliferation, hypoxic or perivascular regions. In some embodiments, the parameters or regions in the TME are used to precisely deliver lentivirally encoded therapeutic payloads. In some such embodiments, therapeutic payloads activate and/or enhance immune cell functions within a TME, which is typically impenetrable to such immune cell functions, such as the functions of cytotoxic lymphocytes.

Macrophages make an ideal therapeutic cell type for targeting a microenvironment, such as a TME because they play a central role in the crosstalk between the adaptive and innate immune systems, are efficiently recruited to and retained within the tumor, and survive in the TME even after their polarization toward a pro-inflammatory phenotype (Long K B, Beatty G L. Harnessing the antitumor potential of macrophages for cancer immunotherapy. Oncoimmunology 2013; 2:e26860; Peng J, Tsang J Y, Li D et al. Inhibition of TGF-beta signaling in combination with TLR7 ligation re-programs a tumoricidal phenotype in tumor-associated macrophages. Cancer Lett 2013; 331:239-249; Beatty G L, Chiorean E G, Fishman M P et al. CD40 agonists alter tumor stroma and show efficacy against pancreatic carcinoma in mice and humans. Science 2011; 331:1612-1616; Pyonteck S M, Akkari L, Schuhmacher A J et al. CSF-1R inhibition alters macrophages polarization and blocks glioma progression. Nat Med 2013; 19:1264-1272; all expressly incorporated by reference in their entireties). Furthermore, engineered macrophages may be generated from a subject's monocyte population that is discarded during the preparation of therapeutic T Cell Receptor (TCR) or Chimeric Antigen Receptor (CAR) T cells. Some of the embodiments described herein include the use of engineered primary macrophages for therapeutic purposes, such as the use of genetically manipulated macrophages with vectors including but not limited to HIV1-based lentivirus. Macrophages are refractory to lentiviral transduction because of their expression of a restriction factor, SAMHD1, which depletes the pool of nucleotide triphosphates available for reverse transcription (Lahouassa H, Daddacha W, Hofmann H et al. SAMHD1 restricts the replication of human immunodeficiency virus type 1 by depleting the intracellular pool of deoxynucleoside triphosphates. Nat Immunol 2012; 13:223-228; expressly incorporated by reference in its entirety). Recent development of a lentiviral packaging system that generates virions containing viral protein X (Vpx), an SIV and HIV2-associated protein that induces the degradation of SAMHD1, has made it possible to stably deliver genes to primary human myeloid cells (Bobadilla S, Sunseri N, Landau N R. Efficient transduction of myeloid cells by an HIV-1-derived lentiviral vector that packages the Vpx accessory protein. Gene Ther 2013; 20:514-520; expressly incorporated by reference in its entirety).

### Definitions

As used herein, “microenvironment” can include a localized cellular environment for a population of cells, such as tumor cells, or cells associated with an inflammatory response. In some embodiments, a microenvironment can include an in vivo localized cellular environment. A microenvironment can include surrounding blood vessels, immune cells, fibroblasts, bone marrow-derived inflammatory cells, lymphocytes, signaling molecules or the extracellular matrix (ECM). Conditions within a microenvironment can be characterized by the cells and include, for example, increased or decreased levels of intercellular signaling molecules as compared to levels in a systemic circulation or other compartment of an organism. An example of a microenvironment is a TME.

As used herein, the “tumor microenvironment” (TME) can include the surrounding microenvironment that constantly interacts with tumor cells, which is conducive to allow cross-talk between tumor cells and its environment. A TME plays a role in disrupting the cancer immunity cycle and plays a critical role in multiple aspects of cancer progression. For example, the TME can decrease drug penetration, confer proliferative and anti-apoptotic advantages to surviving cells, facilitate resistance without causing genetic mutations and epigenetic changes, and collectively modify disease modality and distort clinical indices. Without being limiting, the TME can include the cellular environment of the tumor, surrounding blood vessels, immune cells, fibroblasts, bone marrow derived inflammatory cells, lymphocytes, signaling molecules or the extracellular matrix. The tumor environment can include tumor cells or malignant cells that are aided and influenced by the TME to ensure growth and survival. The TME can also include tumor-infiltrating immune cells such as lymphoid and myeloid cells, which can stimulate or inhibit the antitumor immune response and stromal cells such as tumor-associated fibroblasts and endothelial cells that contribute to the tumor's structural integrity. Without being limiting, stromal cells can include cells that make up tumor-associated blood vessels, such as endothelial cells and pericytes, which are cells that contribute to structural integrity (fibroblasts), as well as tumor-associated macrophages (TAMs) and infiltrating immune cells including monocytes, neutrophils (PMN), dendritic cells (DCs), T and B cells, mast cells, and/or natural killer (NK) cells. The stromal cells make up the bulk of tumor cellularity while the dominating cell type in solid tumors is the macrophage. A TME can comprise microniches in which the niches are well perfused and oxygenated or poorly perfused and hypoxic. In the case in which the niche is poorly perfused and hypoxic, the niche can be particularly dangerous to the host as it can harbor resistant tumor cells that can survive a nutrient and oxygen deprived environment. The tumor can influence its surrounding environment to be immunosuppressive by the release of extracellular signals, promoting tumor angiogenesis, for example, by the upregulation of VEGF, and induce peripheral immune tolerance.

As used herein, “nucleic acid” or “nucleic acid molecule” can refer to polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes “peptide nucleic acids,” which comprise naturally-occurring or modified nucleic acid bases attached to a polyamide backbone. Nucleic acids can be either single stranded or double stranded.

As used herein, a “vector” or “construct” can include a nucleic acid used to introduce heterologous nucleic acids into a cell that can also have regulatory elements to provide expression of the heterologous nucleic acids in the cell. Vectors include but are not limited to plasmid, minicircles, yeast, or viral genomes. In some embodiments, the vectors are plasmid, minicircles, viral vectors, DNA or mRNA. In some embodiments, the vector is a lentiviral vector or a retroviral vector. In some embodiments, the vector is a lentiviral vector. As used herein, “Vpx” can include a virion associated protein that is encoded by HIV type 2 and in some simian immunodeficiency virus strains. Vpx can enhance HIV-2 replication in humans. Lentiviral vectors packaged with Vpx protein can led to an increase in the infection of myeloid cells, when used in transfections. In some embodiments, the lentiviral vector is packaged with a Vpx protein. As used herein, “Vpr” protein can refer to Viral Protein R, which is a 14 kDa protein, which plays an important role in regulating nuclear import of the HIV-1 pre-integration complex and is required for virus replication in non-dividing cells. Non-dividing cells can include macrophages, for example. In some embodiments, the lentiviral vector can be packaged with a Vpr protein, or a Vpr protein portion thereof. In some embodiments, the lentiviral vector is packaged with a viral accessory protein. In some embodiments, the viral accessory protein is selected from the group consisting of Vif, Vpx, Vpu, Nef and Vpr. These accessory proteins such as, for example vif, Vpx, vpu or nef interact with cellular ligands to act as an adapter molecule to redirect the normal function of host factors for virus-specific purposes. HIV accessory proteins are described in Strebel et al. (“HIV Accessory Proteins versus Host Restriction Factors, Curr Opin Virol. 2013 December; 3(6): 10.1016/j.coviro.2013.08.004; expressly incorporated by reference in its entirety).

As used herein, “transduction” and “transfection” are used equivalently and the terms mean introducing a nucleic acid into a cell by any artificial method, including viral and non-viral methods.

As used herein, “chimeric receptor” can include a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain. Chimeric receptor can also be referred to as artificial T cell receptors, chimeric T cell receptors, chimeric immunoreceptors, and chimeric antigen receptors (CARs). These receptors can be used to graft the specificity of a monoclonal antibody or binding fragment thereof onto a T-cell with transfer of their coding sequence facilitated by viral vectors, such as a retroviral vector or a lentiviral vector. CARs are genetically engineered T-cell receptors designed to redirect T-cells to target cells that express specific cell-surface antigens. T-cells can be removed from a subject and modified so that they can express receptors that can be specific for an antigen by a process called adoptive cell transfer. The T-cells are reintroduced into the patient where they can then recognize and target an antigen. These CARs are engineered receptors that can graft an arbitrary specificity onto an immune receptor cell. The term chimeric antigen receptors or “CARs” are also considered by some investigators to include the antibody or antibody fragment, the spacer, signaling domain, and transmembrane region. Different components or domains of the CARs described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CARs can, for example, lead to stronger binding affinity for a specific epitope or antigen. In some embodiments, the CARs provided herein comprise a T2A cleavage sequence. An example cleavage sequence is SEQ ID NO:51.

As used herein, a “regulatory element” can include a regulatory sequence, which is any DNA sequence that is responsible for the regulation of gene expression, such as promoters, enhancers, and operators. The regulatory element can be a segment of a nucleic acid molecule, which is capable of or configured to increase or decrease the expression of specific genes within an organism. In some embodiments described herein, a protein is under a control of a regulatory element.

As used herein, a “promoter” can include a nucleotide sequence that directs the transcription of a gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, differentiation-specific elements (DSEs; McGehee et al., Mol. Endocrinol. 7:551 (1993); hereby expressly incorporated by reference in its entirety), cyclic AMP response elements (CREs), serum response elements (SREs; Treisman et al., Seminars in Cancer Biol. 1:47 (1990); expressly incorporated by reference in its entirety), glucocorticoid response elements (GREs), and binding sites for other transcription factors, such as CRE/ATF (O'Reilly et al., J. Biol. Chem. 267:19938 (1992); expressly incorporated by reference in its entirety), AP2 (Ye et al., J. Biol. Chem. 269:25728 (1994); expressly incorporated by reference in its entirety), SP1, cAMP response element binding protein (CREB; Loeken et al., Gene Expr. 3:253 (1993); hereby expressly incorporated by reference in its entirety) and octamer factors (see, in general, Watson et al., eds., Molecular Biology of the Gene, 4th ed. (The Benjamin/Cummings Publishing Company, Inc. 1987; expressly incorporated by reference in its entirety)), and Lemaigre and Rousseau, Biochem. J. 303:1 (1994); expressly incorporated by reference in its entirety). As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known. In some embodiments described herein, a method of making a genetically modified immune cell for modifying a tumor microenvironment (TME) is provided, wherein the method comprises delivering a first vector to an immune cell, wherein the first vector comprises a nucleic acid encoding a protein that induces T-cell proliferation, promotes persistence and activation of endogenous or adoptively transferred NK or T cells and/or induces production of an interleukin, an interferon, a PD-1 checkpoint binding protein, HMGB1, MyD88, a cytokine or a chemokine. In some embodiments, the protein is a fusion of a PD-1 checkpoint binding protein and interferon alpha, interferon beta, or interferon gamma. In some embodiments, the nucleic acid encoding said protein is under the control of a regulatory element. In some embodiments, the regulatory element is a promoter that is inducible by a drug. In some embodiments, the regulatory element is a promoter that is inducible by a steroid, such as a ligand for the estrogen receptor. In some embodiments, the regulatory element is a promoter inducible by tamoxifen and/or its metabolites. In some embodiments, promoters used herein can be inducible or constitutive promoters. Without being limiting, inducible promoters can include, for example, a tamoxifen inducible promoter, tetracycline inducible promoter, or a doxycycline inducible promoter (e.g. tre) promoter. Constitutive promoters can include, for example, SV40, CMV, UBC, EFlalpha, PGK, or CAGG.

As used herein, “operably-linked” can refer to two nucleic acids linked in manner so that one may affect the function of the other. Operably-linked nucleic acids may be part of a single contiguous molecule and may or may not be adjacent. For example, a promoter is operably linked with a protein-coding nucleic acid in a polynucleotide where the two nucleic acids are configured such that the promoter can affect or regulate the expression of a transgene. In some embodiments, a regulatory element, for example a promoter and/or an enhancer, can be operably-linked to a nucleic acid encoding a therapeutic payload.

As used herein, “immune cells” can refer to cells of the immune system that are involved in the protection of infectious disease and protection from cancer cells. In some embodiments described herein, a method of making a genetically modified immune cell for modifying a TME is provided, wherein the method comprises delivering a first vector to an immune cell, wherein the first vector comprises a nucleic acid encoding a protein that induces T-cell proliferation, promotes persistence and activation of endogenous or adoptively transferred NK or T cells and/or induces production of an interleukin, an interferon, a PD-1 checkpoint binding protein, HMGB1, MyD88, a cytokine or a chemokine. In some embodiments, the protein is a fusion of a PD-1 checkpoint binding protein and interferon alpha, interferon beta, or interferon gamma. In some embodiments, the immune cell is a myeloid cell. In some embodiments, the myeloid cell is a macrophage. In some embodiments, the myeloid cell is a microglial cell.

Cancer is associated with uncontrolled or dysregulated cell growth. Cancer can present as malignant tumors or malignant neoplasms having abnormal cell growth, which can invade and spread to other parts of the body. In some embodiments described herein, a method of modulating the suppression of the immune response in a TME of a subject in need thereof e.g., a human is provided, wherein the method comprises administering any one or more of the genetically modified immune cells of any one or more of the embodiments described herein to a subject in need thereof e.g., a human and, optionally, selecting or identifying said subject to receive said genetically modified immune cells and/or measuring a modulation of suppression of the immune response in the TME of said subject after administration of said genetically modified immune cells. Subjects that can be addressed using the methods described herein include subjects identified or selected as having cancer, including but not limited to colon, lung, liver, breast, renal, prostate, ovarian, skin (including melanoma), bone, leukemia, multiple myeloma, or brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer. In some embodiments, the subject that receives one of the therapies described herein is also selected to receive an additional cancer therapy, which can include a cancer therapeutic, radiation, chemotherapy, or a cancer therapy drug. In some embodiments, the cancer therapy drug provided comprises Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

As used herein, “natural killer cells” or NK cells are a type of cytotoxic lymphocyte important to the innate immune system. The role NK cells play is analogous to that of cytotoxic T cells in the vertebrate adaptive immune response. NK cells provide rapid responses to viral-infected cells and respond to tumor formation. The function of NK cells is important to the prevention of de novo tumor growth through a process known as immune surveillance (Dunn et al., Cancer immunoediting: from immunosurveillance to tumor escape. Nat Immunol 3, 991-998 (2002); Langers et al., Natural killer cells: role in local tumor growth and metastasis. Biologics: targets & therapy 6, 73-82 (2012); both references expressly incorporated by reference in their entireties herein).

As used herein, “myeloid cells” can refer to a granulocyte or monocyte precursor cell in bone marrow or spinal cord, or a resemblance to those found in the bone marrow or spinal cord. The myeloid cell lineage includes circulating monocytic cells in the peripheral blood and the cell populations that they become following maturation, differentiation, and/or activation. These populations include non-terminally differentiated myeloid cells, myeloid derived suppressor cells, or differentiated macrophages. Differentiated macrophages include non-polarized and polarized macrophages, resting and activated macrophages. Without being limiting, the myeloid lineage can also include granulocytic precursors, polymorphonuclear derived suppressor cells, differentiated polymorphonuclear white blood cells, neutrophils, granulocytes, basophils, eosinophils, monocytes, macrophages, microglia, myeloid derived suppressor cells, dendritic cells or erythrocytes. For example, microglia can differentiate from myeloid progenitor cells.

As used herein, “treat,” “treating,” “treated,” or “treatment” can refer to both therapeutic treatment and prophylactic or preventative treatment depending on the context.

As used herein, “ameliorate,” “ameliorating,” “amelioration,” or “ameliorated” in reference to a disorder can mean reducing the symptoms of the disorder, causing stable disease, or preventing progression of the disorder, For disorders such as cancer, this can include reducing the size of a tumor, reducing cancer cell growth or proliferation, completely or partially removing the tumor (e.g., a complete or partial response), causing stable disease, preventing progression of the cancer (e.g., progression free survival), or any other effect on the cancer that would be considered by a physician to be a therapeutic.

As used herein, “administer,” administering,” or “administered” can refer to all means of introducing the compound, or pharmaceutically acceptable salt thereof, or modified cell composition, to a patient, including, but not limited to, oral, intravenous, intramuscular, subcutaneous, or transdermal.

As used herein, “subject” or “patient,” can refer to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, or humans. In some embodiments, the subject is a cow, sheep, pig, horse, dog, cat, primate or a human.

Certain Polynucleotides

Some embodiments of the methods and compositions provided herein include polynucleotides. In some embodiments, a polynucleotide includes a first nucleic acid comprising a regulatory element operably-linked to a therapeutic payload. In some embodiments, the regulatory element can include a promoter and/or enhancer. In some embodiments the regulatory element is capable of or is configured to induce specific transcription of the therapeutic payload in a cell. For example, the regulatory element may induce transcription of the therapeutic payload in response to a specific stimulus, such as certain a stimulus present in a microenvironment of a cell, and absent in other locations of an organism. In some embodiments, transcription does not occur or is substantially reduced in the absence of the stimulus. For example, in the absence of the stimulus, transcription can be reduced in the absence of the stimulus by at least 50%, 60%, 70%, 80%, 90%, 95%, 98%, 100%, or within a range defined by any two of the foregoing percentages, as compared to the level of transcription in the presence of the stimulus.

In some embodiments, the microenvironment is an in vivo microenvironment, such as a TME, or an inflammation microenvironment.

In some embodiments, the stimulus can include a stimulus endogenous to the microenvironment. Examples of such stimuli include increased or decreased levels of a protein or nucleic acid encoding the protein in the microenvironment as compared to other compartments or locations in an organism, such as a systemic circulation or healthy tissues during homeostasis. In some embodiments, a stimulus can include changes in levels of chemokines, contents of lysed neutrophils, protein or nucleic acid fragments, lipids and fatty acids, sterols, or other metabolic components and byproducts. In some embodiments, the increased or decreased levels of a protein or nucleic acid encoding the protein can include signaling molecules, such as cytokines or chemokines. Examples of signaling molecules include vascular endothelial growth factor (VEGF), transforming growth factor (TGF), a tumor necrosis factor (TNF), IL-6, an interferon, C3b, or macrophages colony-stimulating factor (M-CSF). In some embodiments, an endogenous stimulus can be a decreased level of oxygen in the microenvironment as compared to other compartments or locations in an organism, such as a systemic circulation, or healthy tissues during homeostasis. In some embodiments, an endogenous stimulus can be an increased level of a reactive oxygen species (ROS) in the microenvironment as compared to other compartments or locations in an organism, such as a systemic circulation, or healthy tissues during homeostasis.

In some embodiments, the stimulus can be generated from an activated chimeric receptor in a cell. In some embodiments, the chimeric receptor can be activated by endogenous stimuli presented in a microenvironment. More examples of endogenous stimuli of a microenvironment include activated immune cells.

In some embodiments, the regulatory element comprises a promoter, an enhancer, or a functional fragment thereof capable of or configured to induce transcription of a payload in a cell derived from a gene selected from APOE, C1QA, SPP1, RGS1, C3, HSPA1B, TREM2, A2M, DNAJB1, HSPB1, NR4A1, CCL4L2, SLC1A3, PLD4, HSPA1A, OLR1, BIN1, CCL4, GPR34, EGR1, HLA-DQA1, FCGR3A, VSIG4, LILRB4, CSF1R, HSPA6, TUBA1B, BHLHE41, GSN, JUN, CX3CR1, HLA-DQB1, HSPE1, FCGR1A, CCL3L1, OLFML3, ADAM28, YWHAH, GADD45B, SLCO2B1, HSP90AA1, HSPA8, RNASET2, HLA-DPA1, CDKN1A, CD83, HAVCR2, DDIT4, C3AR1, HSPD1, LGMN, TMIGD3, CD69, IFI44L, SERPINE1, HLA-DMA, ALOX5AP, EPB41L2, HSP90AB1, HSPH1, RHOB, CH25H, FRMD4A, CXCL16, FCGR1B, HLA-DMB, GPR183, HLA-DPB1, SLC2A5, EGR2, ID2, RGS10, APBB1IP, EVL, CSF2RA, SGK1, FSCN1, BEST1, ADORA3, IFNGR1, MARCKS, MT2A, SRGAP2, ARL5A, ADGRG1, HMOX1, RHBDF2, ATF3, SOCS6, NR4A3, PLK3, APMAP, AKR1B1, UBB, HERPUD1, CTSL, BTG2, IER5, LPAR6, USP53, ST6GAL1, ADAP2, HTRA1, KCNMB1, DNAJA1, LPCAT2, ZFP36L1, CCL3, BAG3, TMEM119, LTC4S, EGR3, FCGBP, ABI3, IFNγ, TNFα, IFNα, IL-6, or IL-12. Exemplary promoter sequences useful with embodiments provided herein are listed in TABLE 1. In some embodiments, the regulatory element can include a hypoxia response element (HRE), a SRC binding element, a SMAD 2 response element, a SMAD 3 response element, an ATF binding site, a STAT 2 binding site, a CBP binding site, or a SYK binding element. An example of an HRE from an EPO gene is SEQ ID NO:55 “CCGGGTAGCTGGCGTACGTGCTGCAG”. Another example of an HRE is SEQ ID NO:44

In some embodiments, the regulatory element can include a constitutive promoter. In some such embodiments, additional elements can be inducible to a stimulus presented in a microenvironment. Examples of constitutive promoters include a MiniTK promoter, or an EF1a promoter.

In some embodiments, the polynucleotide includes a second nucleic acid encoding the therapeutic payload. In some embodiments, the therapeutic payload can encode a nucleic acid or protein to treat or ameliorate a microenvironment, such as a TME or inflammatory microenvironment. In some embodiments, the therapeutic payload can encode a nucleic acid or protein that induces T-cell proliferation, promotes persistence and activation of endogenous or adoptively transferred NK or T cells and/or induces production of an interleukin, an interferon, a PD-lcheckpoint binding protein, HMGB1, MyD88, a cytokine or a chemokine. In some embodiments, the therapeutic payload can include an interleukin. Examples of interleukins include IL-10 and IL-12, IL-1, IL-6, IL-7, IL-15, IL-2, IL-18 or IL-21. In some embodiments, a therapeutic payload can encode TGFBRII, interferon alpha, interferon beta, interferon gamma, or TNF-alpha. In some embodiments, the therapeutic payload can encode a chemokine. Examples of chemokines include chemokine comprises CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13 or CXCL15.

In some embodiments, the therapeutic payload can encode a nucleic acid or protein that can modulate an immune response. As used herein, “modulate an immune response” can include an adjustment of an immune response to a desired level, such as, for example, in immunopotentiation, immunosuppression or induction of immunological tolerance. In the embodiments, the therapeutic payload can encode an immunomodulator. Examples of immunomodulators include interleukins, cytokines, immunomodulatory antibodies, or chemokines. More examples of immunomodulators include IL-2, G-CSF, Imiquimod, CCL3, CCL26, CSCL7, TGFBRII, IL-1, IL-6, IL-7, IL-15, IL-2, IL12, IL-18, IL21, interferon alpha, interferon beta, interferon gamma, PD-1 checkpoint binding inhibitor, CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13 or CXCL15.

Certain Vectors

Some embodiments of the methods and compositions provided herein include vectors comprising polynucleotides disclosed herein. In some embodiments, the vector comprises a viral vector. In some embodiments, the vector is a lentiviral vector or a retroviral vector. In some embodiments, the vector is a lentiviral vector. In some embodiments, the lentiviral vector can be packaged with a Vpr protein, or a Vpr protein portion thereof. In some embodiments, the lentiviral vector is packaged with a viral accessory protein. In some embodiments, the viral accessory protein is selected from the group consisting of Vif, Vpx, Vpu, Nef and Vpr. In some embodiments, a vector can include a polynucleotide encoding a chimeric receptor.

Certain Cells

Some embodiments of the methods and compositions provided herein include cells. In some embodiments, a cell can include a polynucleotide and/or a vector disclosed herein. For example, a cell can include a polynucleotide comprising a first nucleic acid comprising a regulatory element operably-linked to a therapeutic payload. In some such embodiments, the regulatory element is capable of or is configured to induce transcription of a therapeutic payload in a cell. In some embodiments, a cell can include a polynucleotide encoding a chimeric receptor. In some embodiments, a cell can include a chimeric receptor protein. In some embodiments, a cell can include a polynucleotide comprising a first nucleic acid comprising a regulatory element operably-linked to a therapeutic payload, such as a regulatory element, which is capable of or is configured to induce specific transcription of a therapeutic payload in the cell, and a polynucleotide encoding a chimeric receptor. In some such embodiments, the chimeric receptor provides a stimulus to induce specific transcription of the first nucleic acid comprising a regulatory element operably-linked to a therapeutic payload.

In some embodiments, the cell is an immune cell. In some embodiments, the cell is a myeloid cell. In some embodiments, the cell is selected from a basophil, neutrophil, eosinophil, or a monocyte. In some embodiments, the cell is a macrophage. In some embodiments, the cell is prepared by contacting a monocyte with GM-CSF to obtain a macrophage. In some embodiments, the cell is a lymphoid cell. In some embodiments, the cell is selected from a natural killer cell, or a T cell. In some embodiments, the cell is mammalian. In some embodiments, the cell is human. In some embodiments, the cell is an ex vivo cell. In some embodiments, the cell is an in vivo cell. In some such embodiments, the in vivo cell can include a genetically modified cell, such as a cell provided for therapy.

Some embodiments include the preparation of cells provided herein. Some such embodiments include introducing a polynucleotide provided herein into a cell. In some embodiments, a polynucleotide comprising a first nucleic acid comprising a regulatory element operably-linked to a therapeutic payload is introduced into a cell. In some embodiments, a polynucleotide encoding a chimeric receptor is introduced into a cell. In some embodiments, a polynucleotide comprising a first nucleic acid comprising a regulatory element operably-linked to a therapeutic payload, such as a regulatory element, which is capable of or configured to induce specific transcription of the therapeutic payload in the cell, and a polynucleotide encoding a chimeric receptor are both introduced into a cell.

Certain Chimeric Receptors

Some embodiments of the methods and compositions provided herein include chimeric receptors. In some embodiments, a chimeric receptor in a cell is activated, and the activated chimeric receptor induces transcription for one or more genes endogenous to the cell. An example embodiment is depicted in FIG. 17A. In some embodiments, a chimeric receptor in a cell can be activated, and the activated chimeric receptor can provide a stimulus to induce specific transcription of a polynucleotide provided herein. An example embodiment is depicted in FIG. 17B. In some such embodiments, the polynucleotide can comprise a first nucleic acid comprising a regulatory element operably-linked to a therapeutic payload.

In some embodiments, a chimeric receptor comprises an extracellular binding domain, a transmembrane domain, and an intracellular signaling domain. In some embodiments, the extracellular binding domain, the transmembrane domain, or the intracellular signaling domain is derived from a receptor selected from a LILRB receptor, a CD115 receptor, a M-CSF receptor; CXCR4; Neuropilin (NRP2); Epidermal Growth Factor receptor; Vascular Endothelial Growth Factor receptor 2; Transforming Growth Factor beta receptor 2; Tumor necrosis factor alpha receptor; Interleukin 6 receptor; Interferon gamma receptor 2; Granulocyte-macrophages colony-stimulating factor receptor subunit alpha; Toll Like receptor 4; Cytokine receptors; TGFb; GM-CSF; IL-6; IL-4; IL-1beta; IL-13; IL-10; IFN-alpha, beta, gamma; Chemokine receptors; CCR1-10; CXCR1, 2, 3, 4, 5, 6; Growth Factor receptor; PDGF; VEGF; EGF; LPS receptor; LDH receptor; MDH receptor; CpG receptor; ssRNA receptor; or a Folate receptor.

Example sequences of components of chimeric receptors are listed in TABLE 2, which include certain example sequences for extracellular, transmembrane, and cytoplasmic domains. In some embodiments, these extracellular, transmembrane, and cytoplasmic domains can be used as modular subunits to create a chimeric receptor. In some such embodiments, the chimeric receptor provides a stimulus to regulate endogenous gene expression, and/or provide a stimulus to induce specific transcription for a polynucleotide provided herein.

In some embodiments, a chimeric receptor provides a stimulus in response to an immune microenvironment signal, such as the presence of soluble factors (chemokines, cytokines, growth factors, nucleic acids, or metabolic enzymes, etc.), or the presence of surface proteins. The receptors listed in TABLE 2 include receptors, which are typically expressed in tumor-associated immune cells and tumor-associated stromal cells, and which can be induced in certain anti-inflammatory programs.

In some embodiments, a chimeric receptor useful in a cancer therapy can include an extracellular and a transmembrane domain of an anti-inflammatory receptor and can include an intracellular domain of a pro-inflammatory, such that the chimeric receptor in a cell is capable of or is configured to initiate an endogenous, pleiotropic pro-inflammatory gene expression profile. In some embodiments, a chimeric receptor useful in a therapy targeted to autoimmune disorder or an inflammatory disorder can include an extracellular domain of a pro-inflammatory receptor and an intracellular domain of an anti-inflammatory receptor, such that the chimeric receptor in a cell is capable of or is configured to initiate an anti-inflammatory gene expression profile.

Certain Methods of Therapy

Some embodiments of the methods and compositions provided herein include methods of therapy. Some such embodiments can include treating or ameliorating or inhibiting a disorder in a subject comprising administering a cell or population of cell provided herein. In some embodiments, the disorder can include a cancer, or an inflammatory disorder or disease. In some embodiments, the subject is mammalian. In some embodiments, the subject is human.

In some embodiments the cancer comprises a solid tumor. In some embodiments the cancer is selected from a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcoma, neuroblastoma, prostate cancer, or ovarian cancer. In some embodiments the cancer is a glioblastoma.

In some embodiments, the disorder includes an inflammatory disorder or disease, and can include a site of inflammation. Examples of disorders and diseases that include sites of inflammation, which respond to administration of one or more of the compositions provided herein include cancer, atherosclerosis, or ischemic heart disease. More examples include acne vulgaris, asthma, certain autoimmune diseases, certain autoinflammatory diseases, celiac disease, chronic prostatitis, colitis, diverticulitis, glomerulonephritis, hidradenitis suppurativa, certain hypersensitivities, certain inflammatory bowel diseases, interstitial cystitis, lichen planus, mast cell activation syndrome, mastocytosis, otitis, pelvic inflammatory disease, reperfusion injury, rheumatic fever, rheumatoid arthritis, rhinitis, sarcoidosis, transplant rejection, or vasculitis.

In some embodiments, a therapy can include the use of autologous cells. In some embodiments, a therapy can include the use of allogeneic cells. In some embodiments, the therapy can include direct injection into a microenvironment, such as a tumor or a site of inflammation. In some embodiments, the therapy can include intravenous administration.

In some embodiments, the tumor can include a tumor bed. A tumor bed can include vascular and stromal tissue that surrounds a cancerous tumor and provides it with oxygen, growth factors, and nutrients. Accordingly, the utility of embodiments of the invention includes non-surgically addressed tumors and other immune suppressive conditions and aspects described herein provide off-the-shelf, ready to administer, allogeneic macrophages products tailored to specific conditions, which support other forms of immunotherapy. In some embodiments of the methods described herein, the genetically modified cells or compositions are injected directly into the tumor beds. In some embodiments, 1×10⁵-2×10⁷ genetically modified cells are injected into a tumor bed. In some embodiments, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷,3×10⁷, 4×10⁷, 5×10⁷, 6×10⁷, or 7×10⁷ genetically modified cells or an amount, which is within a range defined by any two of the aforementioned values are injected into a tumor bed. In some embodiments, the genetically modified cells or compositions are injected within a 1, 2, 5, 10, 20, 30, 40, 50, 60, 70, 80, 90, 100, 110, 120, 130, 140, 150, 160, 170, 180, 190 or 200 mm radius of the tumor bed, or within a radius that is within a range defined by any two of the aforementioned distances.

Certain Kits and Systems

Some embodiments of the methods and compositions provided herein include kits. Some such embodiments include a polynucleotide provided herein. Some such embodiments can include a vector comprising a polynucleotide provided herein. In some embodiments, a kit can include a polynucleotide comprising a first nucleic acid comprising a regulatory element operably-linked to a therapeutic payload. In some such embodiments, the regulatory element is capable of or configured to induce specific transcription of the therapeutic payload in a cell. In some embodiments, a kit can include a polynucleotide encoding a chimeric receptor provided herein. In some embodiments, a kit can include a first polynucleotide comprising a first nucleic acid comprising a regulatory element operably-linked to a therapeutic payload, such as a regulatory element capable of or configured to induce specific transcription of the therapeutic payload in the cell, and a second polynucleotide encoding a chimeric receptor provided herein. In some such embodiments, the chimeric receptor can provide a stimulus to induce specific transcription of the first nucleic acid comprising a regulatory element operably-linked to a therapeutic payload.

Some embodiments of the methods and compositions provided herein include systems. Some such embodiments include a polynucleotide provided herein. Some such embodiments can include a vector comprising a polynucleotide provided herein. In some embodiments, a system can include a polynucleotide comprising a first nucleic acid comprising a regulatory element operably-linked to a therapeutic payload. In some such embodiments, the regulatory element is capable of or configured to induce a specific transcription of a therapeutic payload in a cell. In some embodiments, a system can include a polynucleotide encoding a chimeric receptor provided herein. In some embodiments, a system can include a first polynucleotide comprising a first nucleic acid comprising a regulatory element operably-linked to a therapeutic payload, such as a regulatory element capable of or configured to induce specific transcription of the therapeutic payload in the cell, and a second polynucleotide encoding a chimeric receptor provided herein. In some such embodiments, the chimeric receptor can provide a stimulus to induce specific transcription of the first nucleic acid comprising a regulatory element operably-linked to a therapeutic payload.

### EXAMPLES

### Example 1—Hypoxia-Induced Gene Expression in 293T Cells In Vitro

Transgenes depicted in FIG. 1 were constructed, and included: (A) a CD19t construct encoding a truncated CD19 (CD19t); (B) an EF1 construct including an eF 1a promoter and a GFP/luciferase reporter gene; (C) a miniTK construct including a minimal thymidine kinase promoter and a GFP/luciferase reporter gene; and (D) an HRE miniTK construct including a series of three hypoxia response elements (HRE), a minimal thymidine kinase promoter and a GFP/luciferase reporter gene (HRE MiniTK eGFP:ffluc-t2a-CD19t) The HRE included the sequence SEQ ID NO:44. The CD19t provided a marker for selection and/or transduction efficiency.

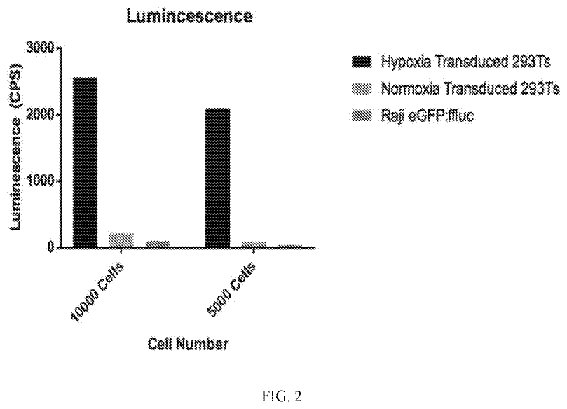

Human 293T cells (human embryonic kidney cell line) or Raji cells (human lymphoblast-like cell line) were transduced with construct (D) and incubated for 20 hr in a hypoxia chamber. Control transduced cells were incubated at normal levels of oxygen (normoxia). Levels of luminescence were determined for the transduced cells. As shown in FIG. 2, hypoxic conditions induced expression of the luciferase reporter gene at levels significantly greater than cells incubated at normal levels of oxygen. FIG. 3 depicts a graph of levels of variability for the level of luminescence in 293T cells or Raji cells transduced with a transgene and incubated for 20 hours in a hypoxia chamber, control transduced cells were incubated at normal levels of oxygen (normoxia).

### Example 2—Hypoxia-Induced Gene Expression in Primary Human Macrophages In Vitro

Primary human macrophages were obtained by treating monocytes with GM-CSF. Differentiated macrophages were plated at 1×10³ cells/well in 96-well plates. Cells were transduced with transgenes depicted in FIG. 1. At day 7, test plates were incubated in a hypoxia chamber for 24 hr (hypoxia conditions: 5% O₂, 10% CO₂, 85% N₂). At day 8, levels of luciferase expression was determined for the transduced cells. An example sequence for a HRE, MiniTK and luciferase construct is depicted in TABLE 3.

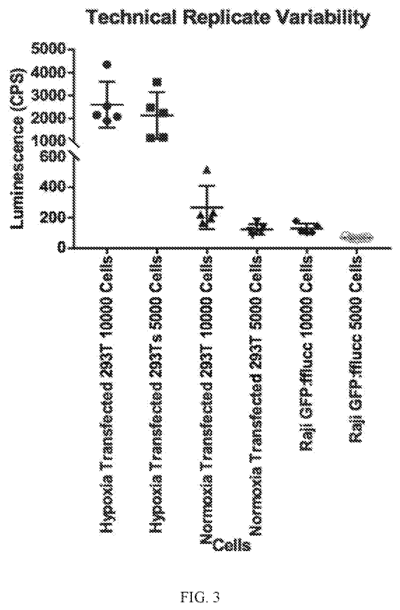

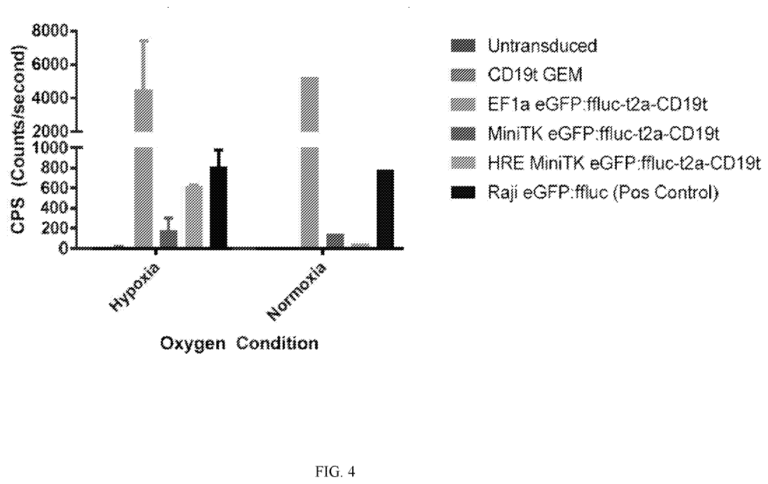

Levels of luciferase activity were measured. As shown in FIG. 4, hypoxia induced expression from a transgene including hypoxia response elements in primary human engineered macrophages as measured by luciferase activity. The relative increase in luciferase activity expressed from a transgene including hypoxia response elements was about 10-fold in hypoxic conditions, compared to non-hypoxic conditions (FIG. 5).

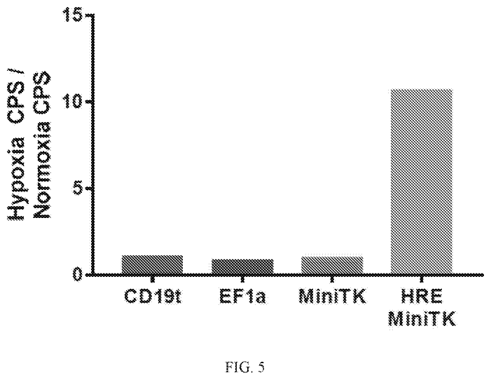

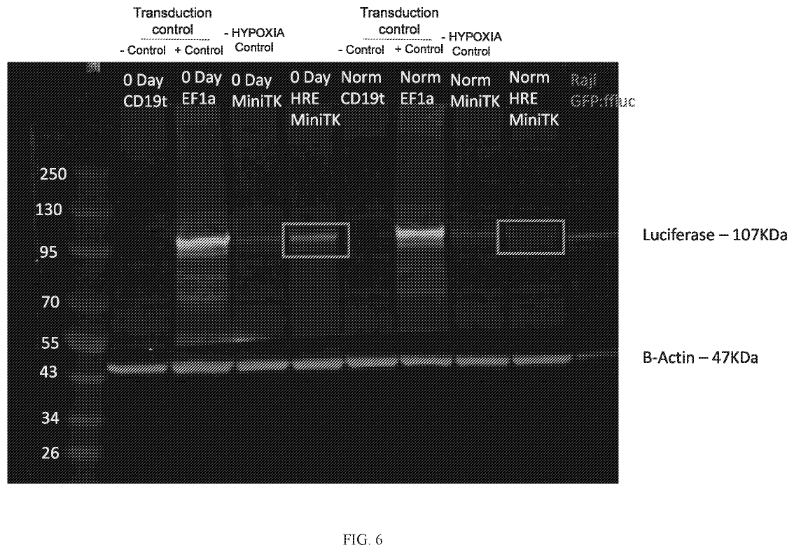

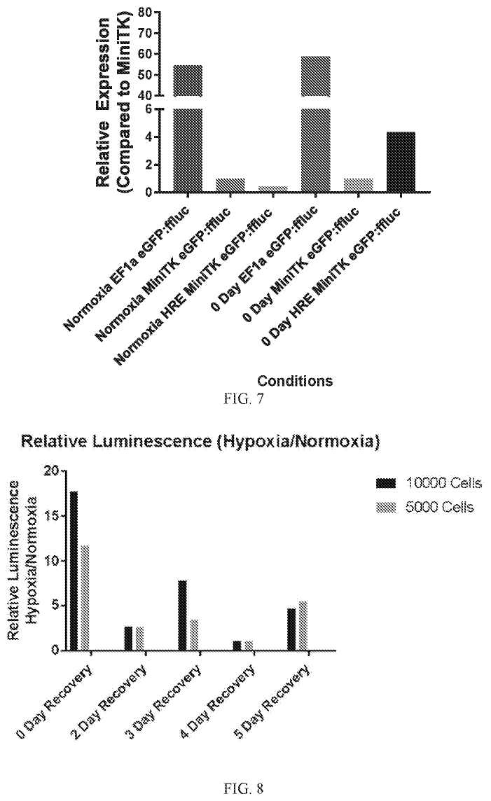

Levels of luciferase protein were measured. The levels of luciferase protein expression was measured at Day 0. FIG. 6 depicts a Western blot prepared from protein extracts from primary human macrophages transduced with various transgenes before incubation in a hypoxia chamber. FIG. 7 depicts a graph of the relative levels of luciferase protein expression in primary human macrophages transduced with various transgenes before incubation in a hypoxia chamber. Some luciferase protein was detected from a transgene including hypoxia response elements, and this was about 4 fold greater than levels detected in cells transduced with a control transgene (construct (C)—miniTK eGFP:ffluc-t2a-CD19t).

### Example 3—Post-Hypoxia Transgene Expression in 293T Cells

Human 293T cells were transduced with a transgene comprising hypoxia response elements and a luciferase reporter gene and incubated in a hypoxia chamber. The relative levels of luciferase activity were measured for transduced cells incubated in a hypoxia chamber compared to transduced cells incubated at normal conditions at day 0, 2, 3, 4, and 5 after hypoxic conditions were removed. As shown in FIG. 8, the relative levels of luciferase activity decreased after removal of the cells from a hypoxia chamber.

### Example 4—Post-Hypoxia Transgene Expression in Primary Human Macrophage

Transgene expression was measured after treatment under hypoxic conditions. GM-CSF differentiated and transduced monocyte derived macrophages were lysed. RNA was isolated from the cell extracts, and cDNA prepared from the isolated RNA. Levels of luciferase were measured relative to (3-actin loading controls. Levels were measured at days 0, 1, 2, 3 and 5, after removal of hypoxic conditions. Relative expression of luciferase transcripts were measured for human primary human macrophages transduced with: CD19t: a transgene encoding a truncated CD19 (CD19t); eGFP: a transgene including an eF1a promoter and a GFP/luciferase reporter gene (eF1a eGFP:ffluc-t2a-CD190; MiniTK: a transgene including a minimal thymidine kinase promoter and a GFP/luciferase reporter gene (MiniTK eGFP:ffluc-t2a-CD19t); and HRE: a transgene including hypoxia response elements, a minimal thymidine kinase promoter and a GFP/luciferase reporter gene (HRE MiniTK eGFP:ffluc-t2a-CD19t) at days 0, 1 and 2 after removal of hypoxic conditions, or continuation of normal conditions (N).

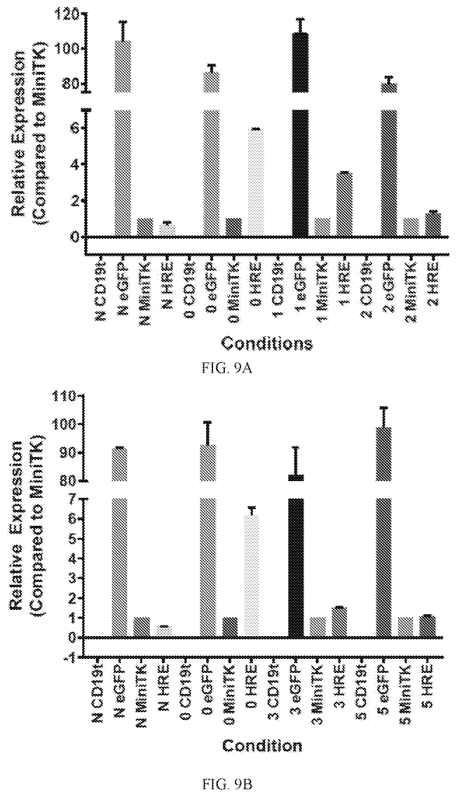

As shown in FIG. 9A and FIG. 9B, cells transduced with CD19t demonstrated no luciferase mRNA expression. Cells transduced with eGFP, a positive control, demonstrated high levels of expression at each time point after removal of hypoxic conditions. Cells transduced with Mini TK, negative control, demonstrated low basal low basal expression at each time point after removal of hypoxic conditions. Cells transduced with HRE demonstrated a reduction in luciferase expression to a level similar to that of cells transduced with MiniTK at 2, 3 and 5 days after removal of hypoxic conditions. Thus, reduction of luciferase expression in cells transduced with the HRE transgene persisted for at least 5 days after removal of hypoxic conditions.

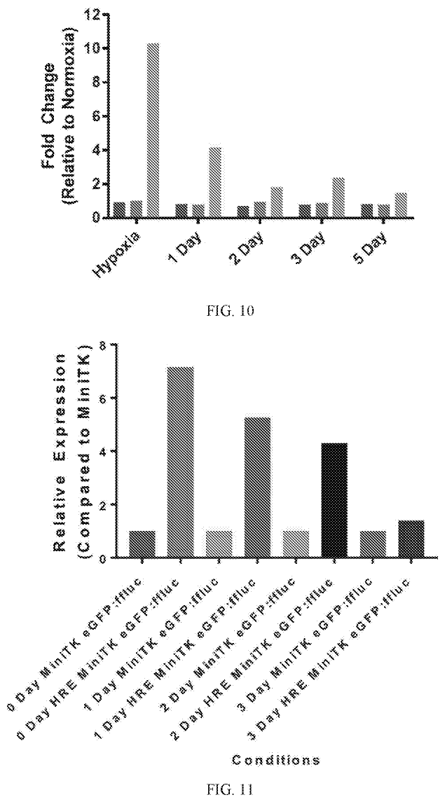

FIG. 10 depicts the results of an additional study and shows that after hypoxic conditions were removed, the relative expression of reporter gene decreased over 5 day period.

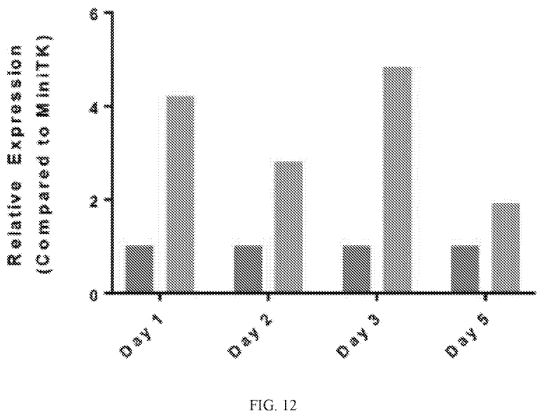

Levels of luciferase protein were measured after removal of hypoxic conditions. As shown in FIG. 11, the relative levels of luciferase protein expression in primary human macrophages transduced with the HRE transgene was reduced over 3 days, to levels comparable to those of the MiniTK control. FIG. 12 depicts an additional study in which luciferase protein levels were measured for 5 days after removal of hypoxic conditions. Thus, HRE was shown to drive expression in response to stimulus, and expression was reduced when the stimulus was removed.

### Example 5—In Vivo Induction of Transgenes with Hypoxia Response Elements

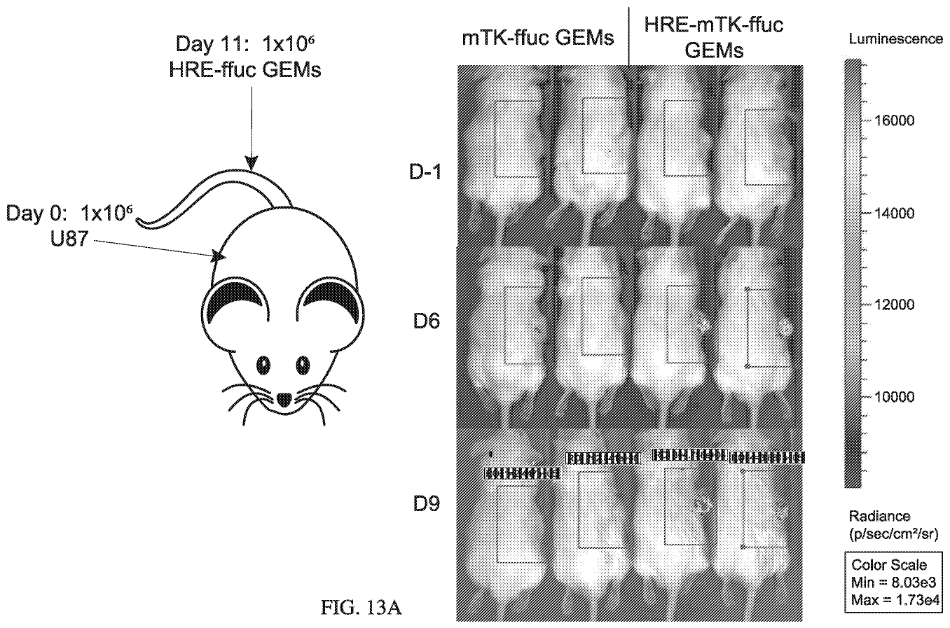

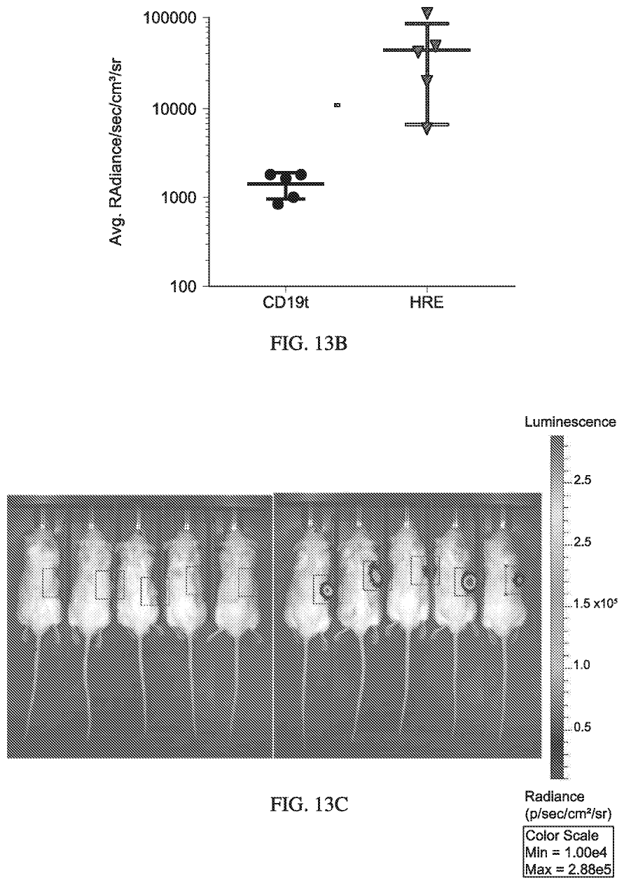

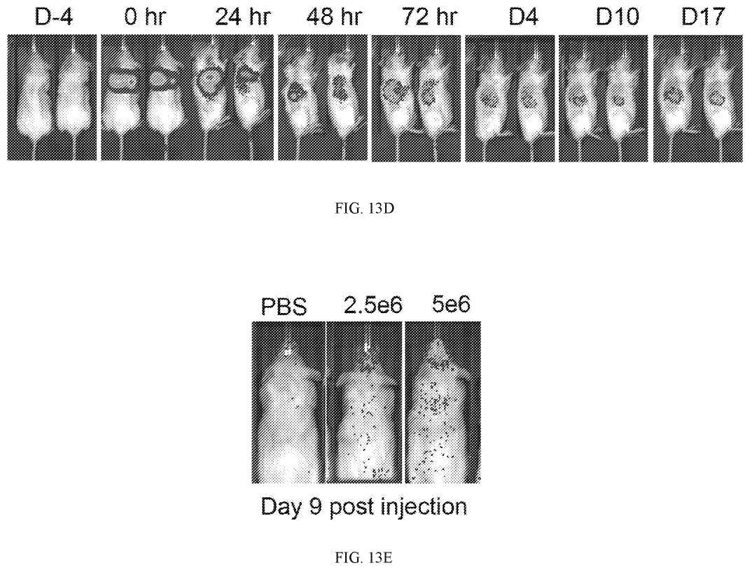

A hypoxic subcutaneous U87 model was developed. Mice were injected at day 0 with 1×10⁶ U87 cells (a human primary glioblastoma cell line) and injected at day 11 with 1×10⁶ genetically engineered macrophages (GEMs) containing a test transgene comprising hypoxia response elements and a luciferase reporter gene (HRE-mTK-ffluc), or a control transgene (mTK-ffluc). See FIG. 13A, left panel. Expression of transgene reporter gene, luciferase, was determined in treated subjects at day 1, day 6 and day 8 (FIG. 13A, right panel).

In mice injected with transgenes containing hypoxia response elements, signals from the transgene reporter genes were detected at days 6 and 9 at locations which corresponded to tumor locations.

In an additional study, GEMs were prepared by transduction with a construct containing a HRE MiniTK eGFP:ffluc-t2a-CD19t, or a construct containing a CD19t. Mice were injected subcutaneously at day 0 with 1×10⁶ U87 cells. At day 19, mice were injected with 1×10⁶ GEMs. Levels of luciferase expression were measured. Average radiance was measured at day 2 post-GEM injection (FIG. 13B). Location and level of expression was measured at day 21. As shown in FIG. 13C, mice injected with GEMs containing a HRE MiniTK eGFP:ffluc-t2a-CD19t showed luciferase expression localized to tumor (FIG. 13C, right panel), while mice injected with GEMs containing a CD19t showed no luciferase expression localized to tumor (FIG. 13C, left panel). Mice were imaged daily showing the path of luciferase expressing GEMs, which localize to a flank tumor within 4 days (FIG. 13D).

In another study, mice were injected with 200,000 U87-MG cells intracranially. 10 days later, mice were injected with escalating doses of luciferase expressing GEMs and imaged using bioluminescence; does included 2.5e6 GEMs, and 5e6 GEMs. As shown in FIG. 13E, luciferase expressing GEMS traffic to tumor tissue in a dose dependent fashion.

### Example 6—Hypoxia-Induced IL-21 Expression in Primary Human Macrophages In Vitro

Primary human macrophages were transduced with transgenes containing genes encoding IL-12 and driven by either an eF1a promoter (EF1a); a minimal thymidine kinase promoter (MiniTK); or hypoxia response elements and a minimal thymidine kinase promoter (HRE MiniTK). Transduced cells were incubated under hypoxic conditions, then hypoxic conditions were removed. Levels of expressed IL-12 were measured at day 0, then after removal of hypoxic conditions at days 1, 2, 3, 4, and 5.

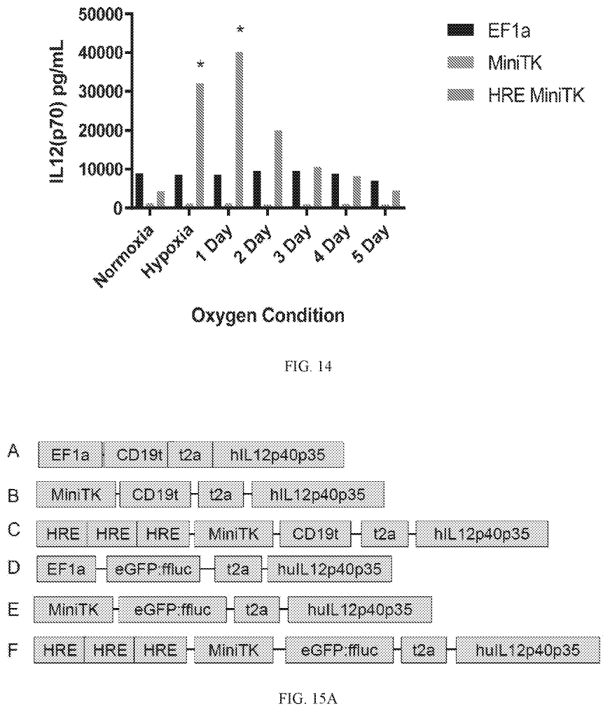

As shown in FIG. 14, hypoxic conditions induced significant IL-12 expression in cells transduced with transgenes containing HREs. After removal of hypoxic conditions, the levels of IL-12 expressed by cells transduced with transgenes containing HREs decreased to levels substantially similar to those of cells transduced with control transgenes.

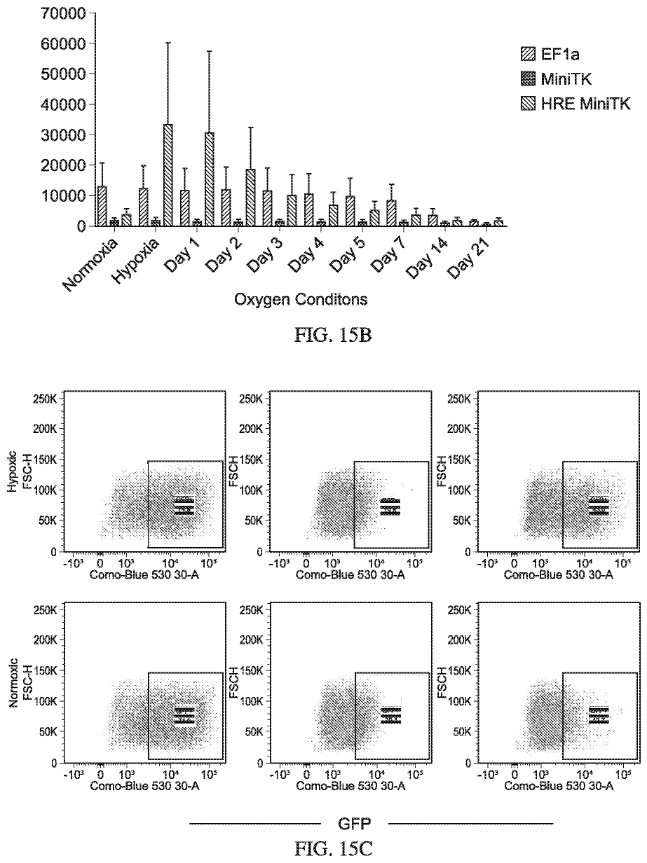

An additional in vitro study was performed in which primary human macrophages were transduced with transgenes containing genes encoding IL-12, and expression was measured for at least 21 days. Transgenes depicted in FIG. 15A were constructed, and included: (A) an EF1a construct including an eF1a promoter (EF1a), and encoding a truncated CD19 (CD19t), and human interleukin 12 p40 and p35 subunits (hIL21p40p35); (B) a miniTK construct including a minimal thymidine kinase promoter (miniTK) and encoding a CD19t, and hIL21p40p35; (C) an HRE miniTK construct including a series of three hypoxia response elements (EIRE), a miniTK promoter and encoding a CD19t, and hIL21p40p35; (D) an EF1a GFP-luciferase construct including an EF1a promoter, and encoding a GFP/luciferase reporter (eGFP:ffluc), and hIL21p40p35; (E) an miniTK GFP-luciferase construct including a miniTK promoter, and encoding eGFP:ffluc and hIL21p40p35; (F) an HRE miniTK GFP-luciferase construct including a series of three HREs, a miniTK promoter and encoding eGFP:ffluc and hIL21p40p35.

Primary human macrophages were transduced with lentiviral vectors containing constructs A, B, or C, and levels of IL-21 secreted into the medium was measured over 21 days and included before cells were placed in hypoxia conditions (normoxia); during hypoxia conditions (hypoxia) for 24 hours; and subsequent days after hypoxia. As shown in FIG. 15B, cells transduced with positive control construct (A) expressed IL-21 over the measured period; cells transduced with negative control construct (B) had minimal IL-21 expression over the measured period; and cells transduced with HRE construct (C) had a substantial IL-21 expression in response to hypoxia conditions, and the level of expression declined after removal of hypoxia conditions.

Primary human macrophages were transduced with lentiviral vectors containing constructs D, E, or F. Cells were treated to either hypoxic or normoxic conditions, and sorted according to GFP expression by flow cytometry. In FIG. 15C, left upper and lower panels represent sorted cells transduced with positive control EF1a (construct D); center upper and lower panels represent sorted cells transduced with negative control miniTK (construct E); and right upper and lower panels represent sorted cells transduced with HRE-miniTK (construct F). As shown in FIG. 15C, the HRE-miniTK construct demonstrated inducible expression under hypoxic conditions.

### Example 7—HRE-Driven Expression in Cultured Human Colorectal Tumors

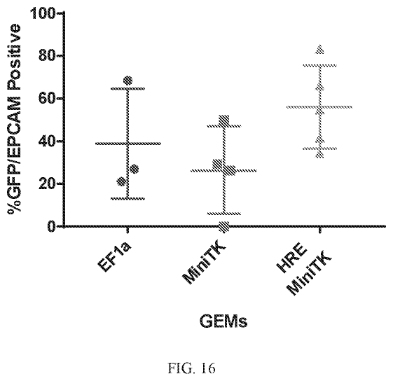

Colorectal carcinoma samples were obtained from patients, and resected to obtain 250 μM thick slices. The slices were cultured in hypoxic conditions with certain 100000 GEMs containing an EF1a construct, a MiniTK construct, or an HRE MiniTK eGFP:ffluc-t2a-CD19t construct. GFP expression in the cultured slices from the GEMs transduced with the constructs was measured. The levels of GFP expression in the cultures were measured a percentage of GFP+ and EPCAM+ cells. As shown in FIG. 16, cultures with GEMS containing the positive control construct (EF1a) or the HRE construct has a greater level of GFP expression that cultures with GEMS containing the negative control construct (miniTK).

### Example 8—Activity of Chimeric Receptors in Primary Human Macrophages In Vitro

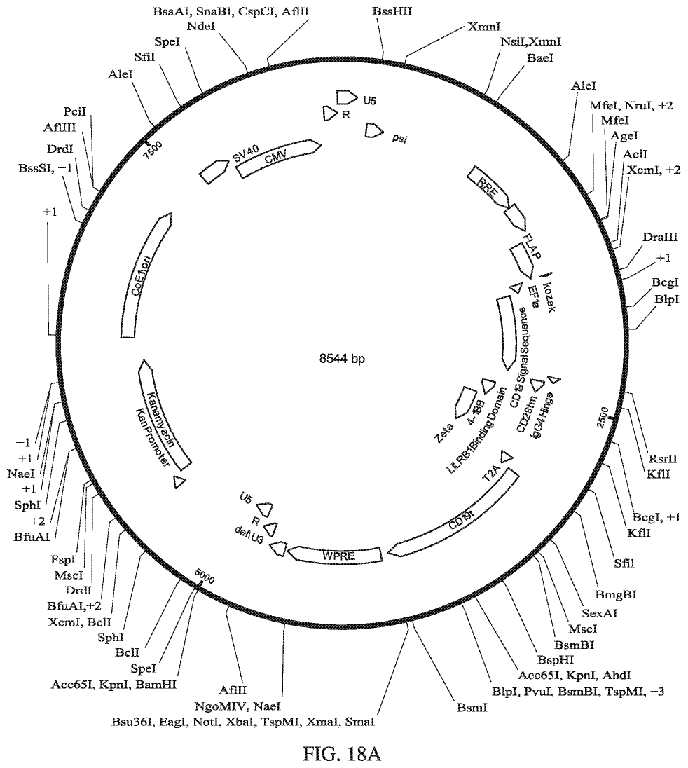

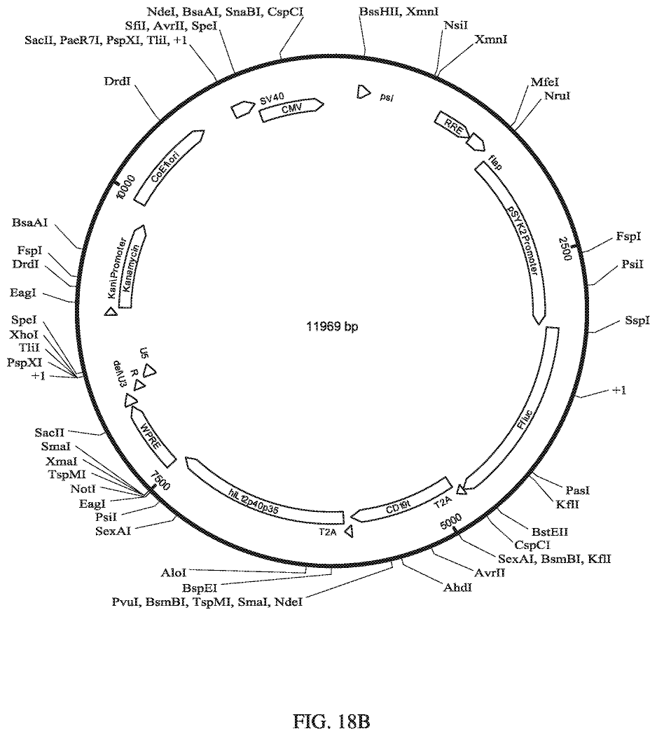

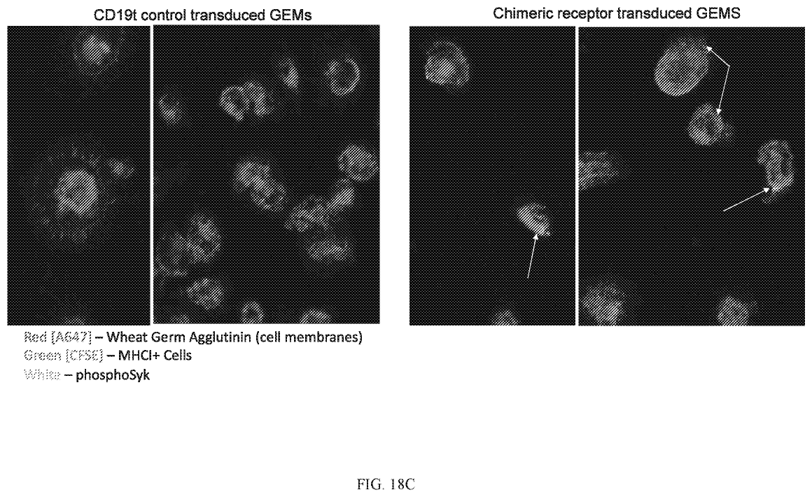

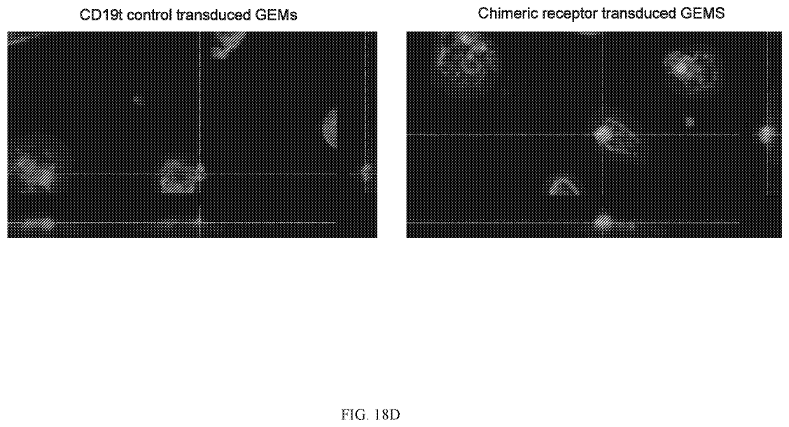

Primary human macrophages were transduced with transgenes encoding chimeric receptors containing a LILRB domain, a transmembrane linker, and a CD3ξ/41BB cytoplasmic domain. As shown in FIG. 17B, binding of MHC class I molecules to the LILRB domain can induce intracellular signaling from the CD3ξ/41BB domain, which can induce phosphorylation of SYK protein. A map of a vector containing an example polynucleotide for the chimeric receptor is shown in FIG. 18A. A map of a vector containing an example polynucleotide for transgene comprising regulatory elements response to phosphorylated Syk is shown in FIG. 18B. Control cells were transduced with a vector encoding a CD19t marker. Transduced cells were contacted with cells expressing MHC class I molecules.

As shown in FIG. 18C, phosphorylated Syk was detected in cells transduced with transgenes encoding chimeric receptors containing a LILRB domain, a transmembrane linker, and a CD3ξ/41BB cytoplasmic domain, and stimulated with cells expressing MHC class I molecules.

Phagocytosis can be a consequence of Syk phosphorylation (Morrissey et al, 2018: doi.org/10.7554/eLife.36688). Transduced cells with contacted with autologous carboxyfluorescein succinimidyl ester (CFSE) labeled T cells. As shown in FIG. 18D, Z stack analysis demonstrated that CFSE labeled cells were within the wheat germ agglutinin (WGA) stained membrane in chimeric receptor transduced macrophages.

### Example 9—Activity of Chimeric Receptors

Activity of chimeric receptors was tested in vitro. Human monocyte derived macrophages were transduced with candidate MCSF-RxTLR4 chimeric receptor-1 or -2 (CR-1 and CR-2). CR-1 is shown in FIG. 19A. CR-2 was substantially similar to CR-1 except it contained a MCSF receptor transmembrane domain, instead of a CD28 transmembrane domain. Nucleotide sequences included in CR-1 and CR-2 are listed in TABLE 4. Control cells were not transduced (UT). Cells were stimulated with LPS/IFNg (10 μg/ml and 100 U/ml) for 48 hours. Supernatant was collected and analyzed for pro-inflammatory cytokines. Stimulated cells containing chimeric receptors expressed TNF-alpha, and IL-12 (FIG. 19B).

Activity of a chimeric receptor was tested in vivo. Human monocyte derived macrophages were transduced with CD19t only or candidate MCSF-RxTLR4 chimeric receptor each upstream of eGFP/ffluc, and injected intratumorally into established U87 GBM intracranial tumors (FIG. 20A). 5 days later, animals were imaged for induction of luciferase expression using IVIS to detect bioluminescence (FIG. 20B).

### Example 10—Identification of Genes Activated in a Tumor Microenvironment

Single cell RNA sequencing was performed on monocytes isolated from peripheral blood, and on patient-matched tumor-associated macrophages obtained from patients undergoing resection of glioblastoma tumors. Over 400 genes were identified that were induced in tumor associated macrophage, but not in the monocytes, which suggested that the promoters of these genes were activated in the TME but are not activated in peripheral circulation.

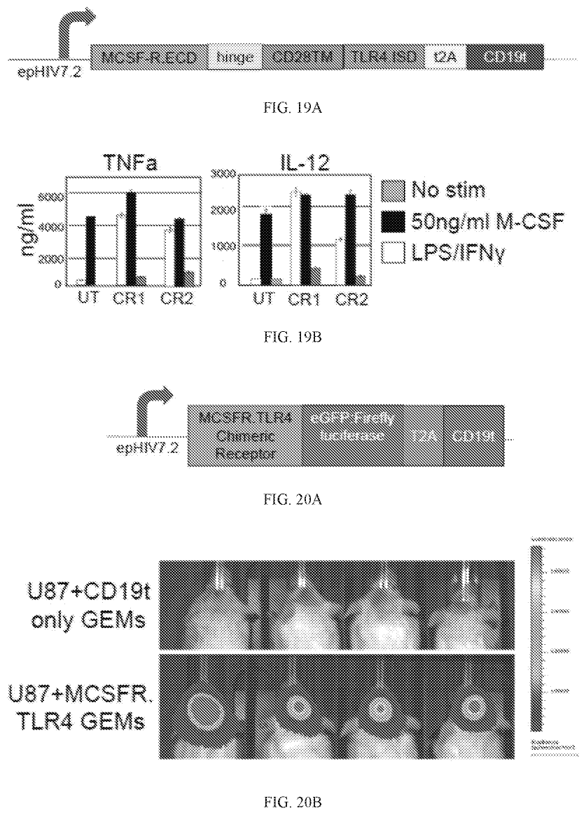

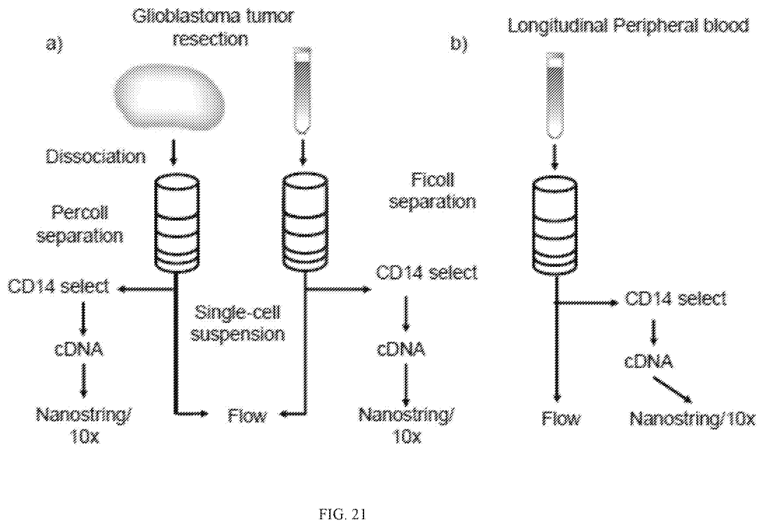

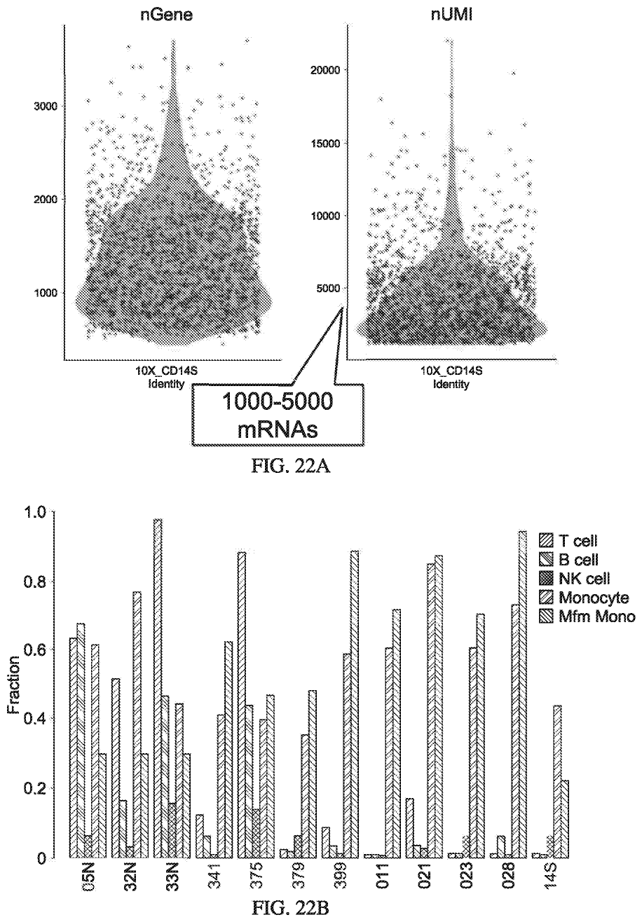

An example study protocol is depicted in FIG. 21. As shown in FIG. 21 (panel A), either glioblastoma (GBM) tumors were resected and dissociated, or peripheral blood were both obtained from a patient, cells of the samples were separated and CD14+ cells were selected, cDNA was generated and sequenced, and analyzed (NanoString Technologies, Inc., Seattle Wash.). As shown in FIG. 21 (panel B), peripheral blood was obtained from a healthy control subject, cells of the samples were separated and CD14+ cells were selected, cDNA was generated and sequenced, and analyzed (NanoString Technologies, Inc., Seattle Wash.). It was found that CD14+ selection increased the likelihood of identifying rare subpopulations expressing genes associated with tumor associated macrophage (TAM). In FIG. 22A, the proportion of cells expressing genes consistent with monocytes and TAM progenitors significantly increased following CD14 magnetic selection that was performed prior to 10× Genomics single cell RNA sequencing, yielding 1000-5000 known mRNAs in circulating monocytes, which are transcriptionally less active than TAMs (FIG. 22B). TABLE 5 lists percentage of CD14+ cells expressing representative TAM-specific genes in samples. Other examples include: CD206, CD209, EGFR, VEGFR, MARCO, VSIG4, HSP5A, HSPA6, HMOX1, LDHA, C5aR, TGFbR1,2,3, MICA, or MICB.

TABLE 5

| Gene | GBM tumor | GBM circulating | Healthy donor |
|---|---|---|---|
| ApoE | 99.9 | 0.7 | 0.1 |
| C1QC | 98.2 | 0.6 | 0.1 |
| C1QB | 97.8 | 0.1 | 5.2 |
| C1QA | 96.8 | 0.7 | 9.5 |
| C3 | 93.8 | 0.0 | 1.0 |
| HSPA1B | 92.2 | 0.5 | 0.6 |
| HSPA1A | 94.1 | 10.0 | 23.1 |
| HSPA6 | 72.5 | 1.3 | 4.3 |
| HSPB1 | 89.6 | 2.4 | 17.5 |
| HSPE1 | 79.7 | 12.1 | 25.6 |
| HLA-DQA1 | 75.4 | 1.1 | 37.9 |
| HLA-DQB1 | 83.1 | 15.2 | 60.5 |
| HLA-DPA1 | 98.2 | 32.9 | 72.1 |
| HLA-DMA | 90.1 | 28.4 | 51.3 |
| HLA-DPB1 | 96.3 | 37.1 | 78.3 |

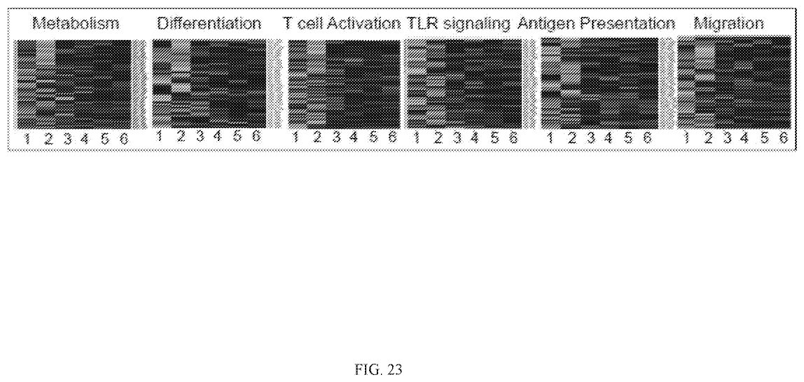

FIG. 23 depicts a nanostring analysis for TAM-associated genes compared to genes expressed in CD14+ monocytes, associated with certain pathways/functions in a myeloid panel of 770 genes, suggesting that patient TAMs differ significantly in pathways known to contribute to pro- and anti-inflammatory immune cell functions, including activation and suppression of cytotoxic immune cells.

### Example 11—Identification of Tumor Associated Macrophage Specific Genes

Additional candidate regulatory elements for expression of a transgene in a tumor microenvironment were identified. Glioma patient tumor samples and peripheral blood were collected from patients undergoing surgical resection. Within 4 hours of collection, samples were dissociated and Percoll gradient purified, and CD14+ cells were selected. Bulk CD14+ cells were lysed and total mRNA sequenced.

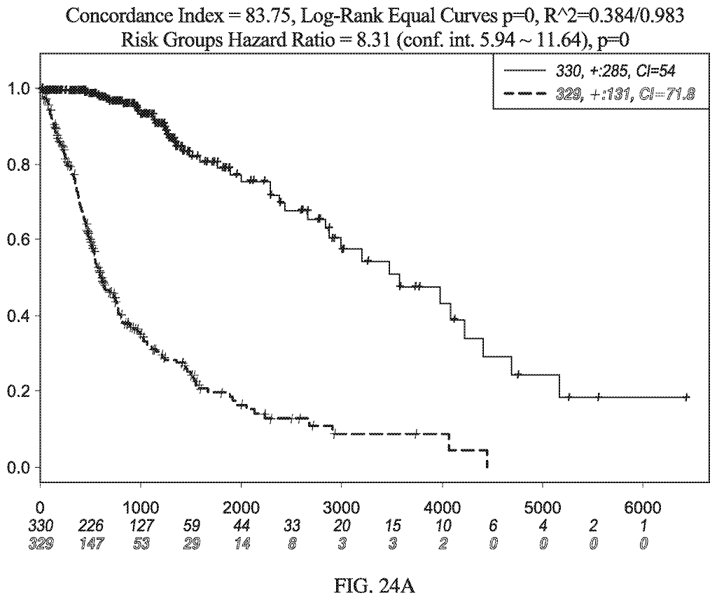

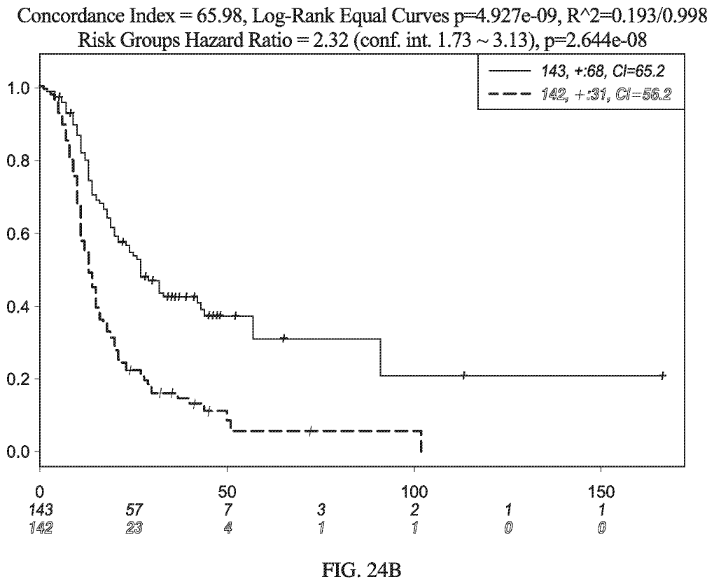

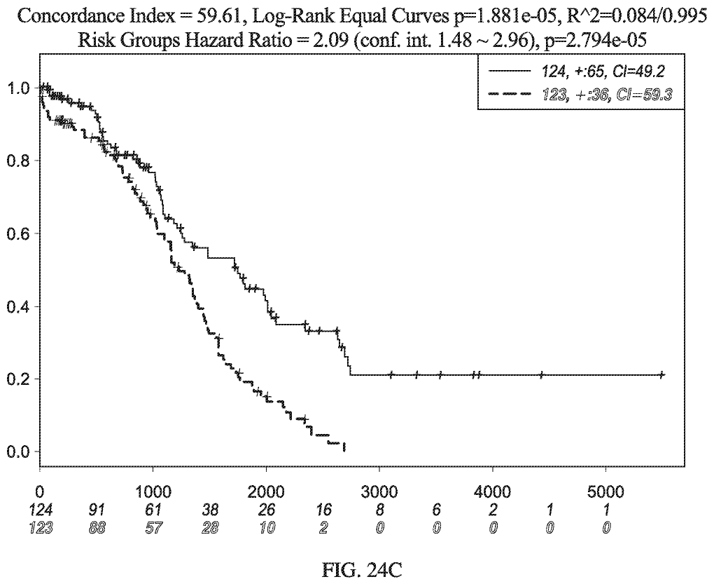

Expressed genes were analyzed to determine an association of gene expression with prognosis and role in disease progression. Expression was correlated with tumor expression in patients having glioma or ovarian tumors, in which the patients had poorer outcomes including shorter periods to relapse and/or survival. FIG. 24A, FIG. 24B, and FIG. 24C each depict a graph for relative level of expression for certain genes in either glioma patients over survival time, ovarian cancer patients over time to relapse, or ovarian cancer patients over survival time, respectively. In each of FIG. 24A-C, the upper line represents more highly expressed genes, while the lower line represents genes with lower levels of expression. Twenty-two genes were identified which included DNAJB1, DNASE2, B3GNT5, RGS1, HMOX1, HSPA5, RNASET2, CAPG, CITED2, NEU1, CYCS, CCL2, HSPA6, JUN, ID2, EGR1, ARID5A, ATF3, ADRB2, CDC42, LSM6, and VSIG4.

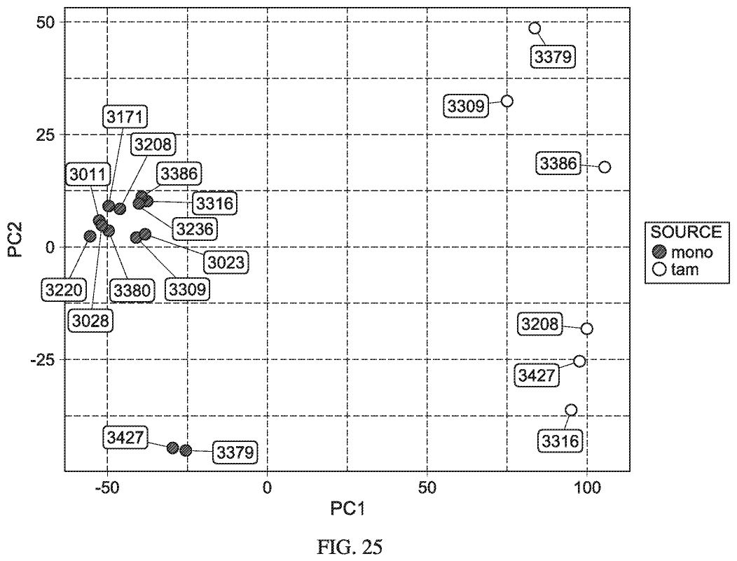

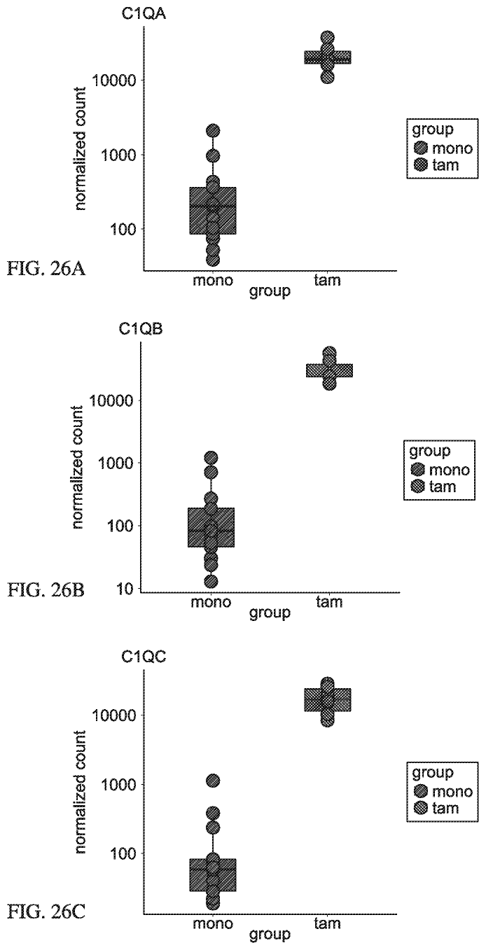

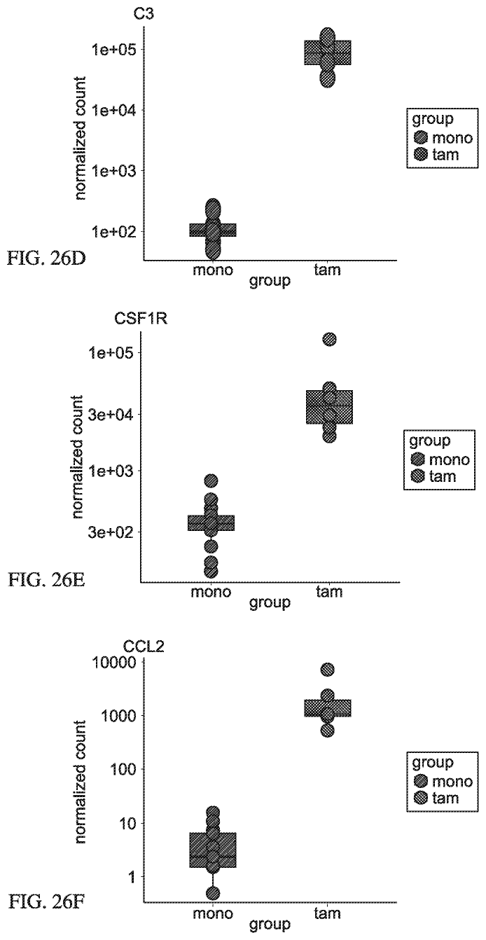

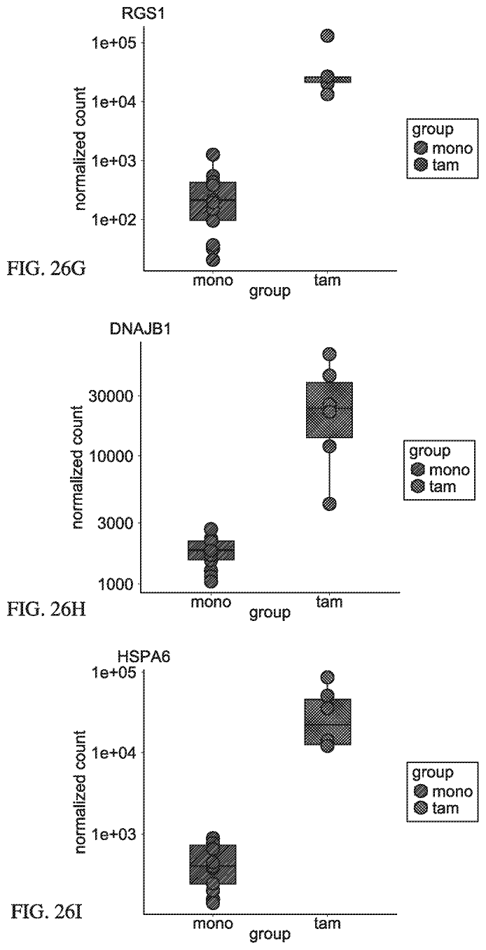

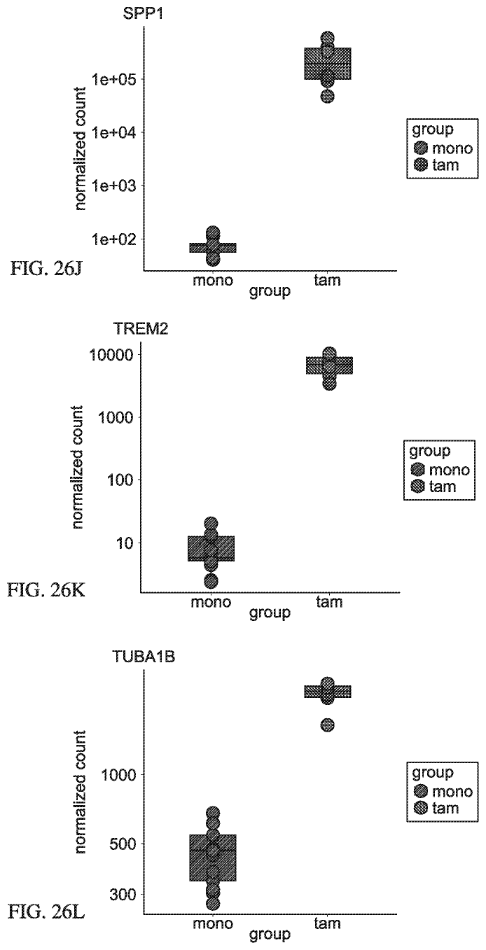

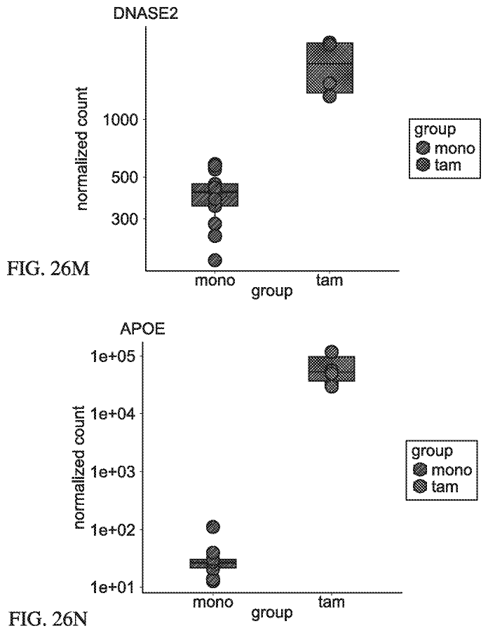

In order to increase confidence of interpretable RNA sequencing data, a principal component analysis was performed for genes in GBM patient TAMS n=6 (closed) and monocytes n=10 (open). The analysis compressed complex data sets to a linear scale. As shown in FIG. 25, principal component 1 (PC1) represented a compression of total gene expression signatures obtained from bulk RNA sequencing, plotted on the X axis, which showed disparate gene expression profiles between TAMs and monocytes, but relatively close associations across patients of TAMs and monocytes to other cells of the same source material (monocytes from peripheral blood and TAMs from patient tumors). Principal component 2 (PC2), shown on the Y axis of FIG. 25, illustrates transcript integrity number for each sample to correct for transcript degradation occurring during the processing and sequencing. This analysis validated the integrity and reproducibility of the material and expression profiles used to derive the plots in FIGS. 26A-26N. In particular, FIGS. 26A-26N depict relative levels of certain gene expression for circulating monocytes and tumor associated macrophages (TAMs), including genes: C1QA, C1QB, C1QC, C3, CSF1R, CCL2, RGS1, DNAJB1, HSPA6, SPP1, TREM2, TUBA1B, DNASE2, and APOE.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and embodiments coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

TABLE 1

| Promoter | |
|---|---|
| (SEQ ID NO) | SEQUENCE |
| APOE Promoter: | GAGGTGCTGGAATCTCATTTCACATGTGGGGAGGGGGCTCCC |
| GH19J044900 | CTGTGCTCAAGGTCACAACCAAAGAGGAAGCTGTGATTAAAA |
| (SEQ ID NO: 01) | CCCAGGTCCCATTTGCAAAGCCTCGACTTTTAGCAGGTGCAT |
| | CATACTGTTCCCACCCCTCCCATCCCACTTCTGTCCAGCCGCC |
| | TAGCCCCACTTTCTTTTTTTTCTTTTTTTGAGACAGTCTCCCTC |
| | TTGCTGAGGCTGGAGTGCAGTGGCGAGATCTCGGCTCACTGT |
| | AACCTCCGCCTCCCGGGTTCAAGCGATTCTCCTGCCTCAGCCT |
| | CCCAAGTAGCTAGGATTACAGGCGCCCGCCACCACGCCTGGC |
| | TAACTTTTGTATTTTTAGTAGAGATGGGGTTTCACCATGTTGG |
| | CCAGGCTGGTCTCAAACTCCTGACCTTAAGTGATTCGCCCACT |
| | GTGGCCTCCCAAAGTGCTGGGATTACAGGCGTGAGCTACCGC |
| | CCCCAGCCCCTCCCATCCCACTTCTGTCCAGCCCCCTAGCCCT |
| | ACTTTCTTTCTGGGATCCAGGAGTCCAGATCCCCAGCCCCCTC |
| | TCCAGATTACATTCATCCAGGCACAGGAAAGGACAGGGTCAG |
| | GAAAGGAGGACTCTGGGCGGCAGCCTCCACATTCCCCTTCCA |
| | CGCTTGGCCCCCAGAATGGAGGAGGGTGTCTGTATTACTGGG |
| | CGAGGTGTCCTCCCTTCCTGGGGACTGTGGGGGGTGGTCAAA |
| | AGACCTCTATGCCCCACCTCCTTCCTCCCTCTGCCCTGCTGTG |
| | CCTGGGGCAGGGGGAGAACAGCCCACCTCGTGACTGGGGGC |
| | TGGCCCAGCCCGCCCTATCCCTGGGGGAGGGGGCGGGACAG |
| | GGGGAGCCCTATAATTGGACAAGTCTGGGATCCTTGAGTCCT |
| | ACTCAGCCCCAGCGGAGGTGAAGGACGTCCTTCCCCAGGAGC |
| | CGGTGAGAAGCGCAGTCGGGGGCACGGGGATGAGCTCAGGG |
| | GCCTCTAGAAAGAGCTGGGACCCT |
| C1QA Promoter: | GTGAGCCAAAGCACCTAGACTTGCTGTTTTTTTAAATGTTGGC |
| GH01J022636 | AAAAGCTAGTGACTAAGTGTTCACCATGTGCCAAGTGTTTTG |
| (SEQ ID NO: 02) | TATGTGCAAGTCATTAAAATGTTATAATAATCAGATGATATA |
| | GGACCCCGTTCCCAGTGTACAGATAAAACTGAGGGCTCAGGA |
| | GGTCAAGTAATTTCCCAAAGACATGCAACTAGGAAGCAGCA |
| | AGTCTAGAAACAGATTGCTGTGCTTCCTTCTCACCAATGCACT |
| | GTTCTTTCTTCTTTGTATTTTCCAAGCTTTTGGCAATGAATTGC |
| | ATGTCACTTTCATAATCATGAAAAGTCCTACAAGGGACAGAT |
| | TTTTTTAAAGATTCCTTCCGCTCTGGTGTTCTGTGCTTCGCTGG |
| | TTCTCAGAACCTCACCCTGCACGGCCTCCACCCCACATCCTCA |
| | CAGGCACTGGCCTCACTGCCCCCACTCCCACACCAGCTGTTG |
| | CTGGGGCAGGACGCCCAATGTCCCAGTCTTGCTGAAGTCTGC |
| | TTGAAATGTCCCTGGTGAGCTTCTGGCCACTGGGGAAGTTCA |
| | GGGGGCAGGTCTGAAGAAGGGGAAGTAGGAAGGGATGTGAA |
| | ACTTGGCCACAGCCTGGAGCCACTCCTGCTGGGCAGCCCACA |
| | GGGTCCCTGGGCGGAGGGCAGGAGCATCCAGTTGGAGTTGA |
| | CAACAGGAGGCAGGTGAGGCCAGAGTCCCAGAGGGAGGGGG |
| | CTGCAGAGTTCTGGGACCCAGGGGAGCTGGCCCAGGAGGCT |
| | GGGCAGCTGAGGCAGGGAGGGAGGGAAATAACTGTGCCTGA |
| | T |
| SPP1 Promoter: | CAAAGCTAAGATTTTGCCTTTAGGAAAGTTTTCTTTCCTAATA |
| GH04J087974 | AAATAGTTTATTTGACAACTATTCTTTTTATTAGGATCATTCA |
| (SEQ ID NO: 03) | TATATTTGCTAAGCAAAGAGTAAATTTATTTTCCTTAAGATTC |
| | AATTTGAATATACTAAGAATATTAAAGCAAGTTAGATAAATT |
| | ACCCAATATATTTGTCAATTTGAAATTTGATAGACATTAGTTG |
| | TTTAATTCAATGGGCAGTTTTGAGCTGCAGTTTATACACACAT |
| | GCATAACAGAGTCACCTTTCAATTATCCATGTTAATAGGAAA |
| | GTGGTTATAGATTTTAGTACACACATTAAAATATGGATACTCT |
| | TCTCTTTTGATAAATCTCATTTCAAATAAAAAAACCAGTCTCA |
| | TAATTATGTATCTGTATCTATTACATCATTGAATTTAGTAAAT |
| | AATGTTTAATATGTATAAGGAAAAACAATGTTATTGACATGA |
| | AGATTATACTCACATATTTGGCTTGAAAATATCTATAAAAAT |
| | AATTTCTGTTGCAAAGTAAGAAATGTTCTTCAGAATGTTATTA |
| | ATCCCTGTGTTAAAAGAGAAATTGGAAGATGCTCACTTTAGC |
| | TCCTAAAAGCCATGGTATGTACTGTGAATGCAAAGATTCTGA |
| | AACTAAATAAAAAGAAAGATAGTAAAAGACTAATGTGCTAT |
| | AAAGGCTAAGGGAAAATAAAAACCCATATATTAATTTTCCCG |
| | GCCATCTTAATTTTCAGACCCTTCCAAGTAAGTCCAACGAAA |
| | GCCATGACCACATGGATGATATGGATGATGAAGATGATGATG |
| | ACCATGTGGACAGCCAGGACTCCATTGACTCGAACGACTCTG |
| | ATGATGTAGATGACACTGATGATTCTCACCAGTCTGATGAGT |
| | CTCACCATTCTGATGAATCTGATGAACTGGTCACTGATTTTCC |
| | CACGGACCTGCCAGCAACCGAAGTTTTCACTCCAGTTGTCCC |
| | CACAGTAGACACATATGATGGCCGAGGTGATAGTGTGGTTTA |
| | TGGACTGAGGTCAAAATCTAAGAAGTTTCGCAGACCTGACAT |
| | C |
| RGS1 promoter: | ACCTTGAGCAGTTTTTCCTCTGATACTGTGACCTTGGTATCAG |
| GH01J192575 | TATGTTTACAAATTATGTCATATTTGTATACAATTAAACATTT |
| (SEQ ID NO: 04) | ATTTATTTATGATTGGTTTTAATTAAACTTTATTTTTTCCTAAT |
| | ATGCAAAATATGCAAAAATCAAAGTTTGATATACTAGTTATA |
| | TTCTTAATAAATATGAAAATAATATTAAAAACCATGCACCCC |
| | TTAAAATCATCCTATGTATTGCCAGTCGATGTCTAGCACACAC |
| | CTTGGAAAATATTAACATGGAAATTTCCTGCCATGGAAATAA |
| | GGGATGAGAATTGGAGTGGGAAAATGTGAAGATAGCCTCTT |
| | ATAACTTTCCCTCCTATCAGATATGTGCAAAATAATTAAACA |
| | AAAATTATTTGTTTACTTTGCCCTGCTTCCTGCAGAAAAGAAG |
| | AGAACTCTTAAAATATTTTGATCAGATGAATTTTATAGTCAA |
| | GGACTATCCAAAGCATGGAGTAAGATACAACTCTATGTGATG |
| | AATTTCTATGTGAGTTTCTATATGATAAATTAATACGCCAAAT |
| | GAAACAGTTTATTGAAACATGCAACTTTTTTAAAATAAACAA |
| | ACATCCTCCAGAACCAAAGACTGATGCTGTTAATGCTTTAGA |
| | CATGTGACATATGTGTGTGTACCTCTGTATGTTTAATTTTCTTT |
| | CTTTCTAATCAAGTGACCTGTTCTCGTTTGGTTAAGTTTCAAC |
| | CACAAGACACCACTGTATGCTTTTTTTTTTTCAAGATACACGT |
| | CACAGCACACCAAGAAAAGGGGAACTTCCAGTGTCTGTGGTA |
| | ACATCACTTGATAAACAGACTCCTTTAAACAGCAAGTGCCTG |
| | TCTGCATTCTACTATATAAAGCAGCAGAGACGTTGACTAGCG |
| | CATATTTGCTAAGAGCACCATGCGCGCAGCAGCCATCTCCAC |
| | TCCAAAGTTAGACAAAATGCCAGG |
| C3 Promoter: | GGAGGAAGACCACCTTTACTGCTATACACATTTGTACCTTTTA |
| (SEQ ID NO: 05) | GATGTTGATCAATATGAATATATTATACACACAGACACACAC |
| | ACAGACACACACACACACACAAACAATACAATTTAATATCCT |
| | AAGAGGATATTGACATTAGACAGGTACAAAAGCTCTAGAAA |
| | TGAGGACTTTCCTCAGTGATGACTTTTTTCACCACCAAAGTCA |
| | CTCAGGCATCCTGACAAGGGTAAGTGAGGGGAGCCTCCTTGG |
| | AAAATAAACTCACTTGGATAGTGAACTCCTGCACATACCTCA |
| | AAGCCCATCTGAAATGTCCCCTCCTACAGGAAGTTTTCCCTG |
| | ACCCTCCAAGAAGCAGAGTTCTATTTCACTGGGGAAAACATT |
| | TCTTCTTCTTCTTTTTTTTCCCTGCCCTGCACATGAGCTAGAAA |
| | ACATTTCATGAAACTGGGAGTTTCTGTGCTGGGCTCTGTCCCT |
| | CCCCCATTCTACTTCCCCTCCCTCAGCATGGAAGCCTCTGGAA |
| | GTGGGGCTCTGACTCCCAGCCTACAGAGAGATTCCTAGGAAG |
| | TGTTCGACTGATAAACGCATGGCCAAAAGTGAACTGGGGATG |
| | AGGTCCAAGACATCTGCGGTGGGGGGTTCTCCAGACCTTAGT |
| | GTTCTTCCACTACAAAGTGGGTCCAACAGAGAAAGGTCTGTG |
| | TTCACCAGGTGGCCCTGACCCTGGGAGAGTCCAGGGCAGGGT |
| | GCAGCTGCATTCATGCTGCTGGGGAACATGCCCTCAGGTTAC |
| | TCACCCCATGGACATGTTGGCCCCAGGGACTGAAAAGCTTAG |
| | GAAATGGTATTGAGAAATCTGGGGCAGCCCCAAAAGGGGAG |
| | AGGCCATGGGGAGAAGGGGGGGCTGAGTGGGGGAAAGGCA |
| | GGAGCCAGATAAAAAGCCAGCTCCAGCAGGCGCTGCTCACTC |
| | CTCCCCATCCTCTCCCTCTGTCCCTCTGTCCCTCTGACCCTGCA |
| | CTGTCCCAGCACCATGGGACCCACCTCAGGTCCCAGCCTGCT |
| | GCTCCTGCTACTAACCCACCTCCCCCTGGCTCTGGGGAGTCCC |
| | ATGTGAGTGGTTATGACTCTACCCACAAACAGGGCT |
| HSPA1B Promoter: | GCCTTAAGGACGGCCTACATACTAAGGAAAATTTTTTTCTAA |
| GH06J031813 | CTCCTGGTTGCAGCTGAGGGGAGCGGCTGAGGGCGGGGACA |
| (SEQ ID NO: 06) | GGGGTGCGGCGGACCCACTGCTCCCATTACCCGACCAGCGCC |
| | TCCCTTCCTCCTTGGATGGGTGCCCCTGTCTTGCTAAGAACTG |
| | CCTGTTTACACAACTGCTTTCCTTGTGAAAATTTAAAGGCTCC |
| | TATTCCCAGTTGTTCTATCCTTGTAGGTTAAAGATTATGTCAA |
| | AAACTATATTGCATTATCTCTTTCCTTCTCCTTCCCATTAAGA |
| | CGGAAAAAACATCCGGGAGAGCCGGTCCGTTTCTCAGGCAG |
| | ACTAGGCCATTAGGTGCCTCGGAGAAAGGACCCAAGGCTGCT |
| | CCGTCCTTCACAGACACAGTCCAATCAGAGTTTCCCAGGCAC |
| | ATCGATGCACCGCCTCCTTCGAGAAACAAGGTAACTTTCGGG |
| | TTCTGGTTGTCTCCAAAGTCATCCGACCAATCTCGCACCGCCC |
| | AGAGCGGGCCCTTCCTGTCAATTACCTACTGAAGGGCAGGCG |
| | GCCAGCATCGCCATGGAGACCAACACCCTTCCCACCACCACT |
| | CCCCCTTTCTCTCAGGGCCCCTGTCCCCTCCAGTGAATCCCAG |
| | AAGACTCTGGAGAGTTCTGAGCAGAGGGCGGCACCCTGCCCT |
| | CTGATTGGTCCAAGGAAGGCTGGGGGGCAGGACGGGAGGCG |
| | AAACCCCTGGAATATTCCCGACCTGGCAGCCTCATCGAGCTT |
| | GGTGATTGGCTCAGAAGGGGAAAGGCGGGTCTCCACGACGA |
| | CTTATAAAAGCCGAGGGGCGCGCGGTCCGGAAAACGGCCAG |
| | CCTGAGGAGCTGCTGCGAGGGTCCGCTTCGTCTTTCGAGAGT |
| | GACTCCCGCGGTCCCAAGGCTTTCCAGAGCGAACCTGTGCGG |
| | CTGCAGGCACCGGCGTGTTGAGTTTCCGGCGTTCCGAAGGAC |
| | TGAGCTCTTGTCGCGGATCCCGTCCGCCGTTTCCAGCCCCCAG |
| | TCTC |
| TREM2 promoter: | TGATCAGGAGTTCAAGCATGTGTGTGCACAAAATAAACACCA |
| GH06J041163 | GTGTGAGCATGTGTGCACAGGAGACACCCAACAGTTCCAAGA |
| (SEQ ID NO: 07) | AGGCTAAACTTGGGCAGAAAATTCCAGGTGGGAGAGAAAAT |
| | TTTCTGTCTTATGGACAGCCCATTTCCCTTTTCCCTTCTAACTA |
| | GGATAATGGTAATAGTTAGTATTTGTTGAATGCTGTGTGTCA |
| | GGCCCTACTGGAAAGCACTTTACCTGTAGGAACCCATATGGT |
| | GCTCCTGATAACCCTTTGCACTATCATTATTCCCACTGTATAG |
| | ATCAGGGAACAGACACAGGTAGGTTTTGGATGTGTGGTTACA |
| | CACCCAGAAAGTCAGGAAGTCTGGCTCCAGAGCTGTGTACTT |
| | AACTGCTGCCACACTACAGGAATGACAGCCCTGGGGGGATG |
| | AACTAAGAGGTGCTGGATGAGGGTCCTGGCCTCTAAAGGCAC |
| | AGCTGTTCTCCAACTCTTGCAAGGCTGAAACCAGAAGATGGC |
| | GGGCATTGCAGCTGGTGGAGGGTCTGAATACAGCTGTGAGGA |
| | TAGTGATCCCTGGGCTAGGCTCTGCAAGGAAACTGAGCAGTG |
| | CAGGGCCTTACCAGCCCCAACCATCTGGGGGCCACCCTGGCT |
| | GGCACCAGCAGGAGGGTGGGCTGGCTTCTCAGAGGTCTGGG |
| | AGACTCAGCCTCCTTCTGCCAGGGCTGCAGTGGCCGACTCCT |
| | CCTCCCCTCTGTCCCCACCCTGCACCGCCTCCAGACCCCAGTC |
| | CTGACTATTGCTTAATCCCCAGGAGCCCAGTTCCTGTGGGCA |
| | GCGCCTGACATGCCTGATCCTCTCTTTTCTGCAGTTCAAGGGA |
| | AAGACGAGATCTTGCACAAGGCACTCTGCTTCTGCCCTTGGC |
| | TGGGGAAGGGTGGCATGGAGCCTCTCCGGCTGCTCATCTTAC |
| | TCTTTGT |
| IFNgamma promoter: | GCAGTGCTGATCTAGAGCAATTTGAAACTTGTGGTAGATATT |
| (SEQ ID NO: 08) | TTACTAACCAACTCTGATGAAGGACTTCCTCACCAAATTGTTC |
| | TTTTAACCGCATTCTTTCCTTGCTTTCTGGTCATTTGCAAGAA |
| | AAATTTTAAAAGGCTGCCCCTTTGTAAAGGTTTGAGAGGCCC |
| | TAGAATTTCGTTTTTCACTTGTTCCCAACCACAAGCAAATGAT |
| | CAATGTGCTTTGTGAATGAAGAGTCAACATTTTACCAGGGCG |
| | AAGTGGGGAGGTACAAAAAAATTTCCAGTCCTTGAATGGTGT |
| | GAAGTAAAAGTGCCTTCAAAGAATCCCACCAGAATGGCACA |
| | GGTGGGCATAATGGGTCTGTCTCATCGTCAAAGGACCCAAGG |
| | AGTCTAAAGGAAACTCTAACTACAACACCCAAATGCCACAAA |
| | ACCTTAGTTATTAATACAAACTATCATCCCTGCCTATCTGTCA |
| | CCATCTCATCTTAAAAAACTTGTGAAAATACGTAATCCTCAG |
| | GAGACTTCAATTAGGTATAAATACCAGCAGCCAGAGGAGGT |
| | GCAGCACATTGTTCTGATCATCTGAAGATCAGCTATTAGAAG |
| | AGAAAGATCAGTTAAGTCCTTTGGACCTGATCAGCTTGATAC |
| | AAGAACTACTGATTTCAACTTCTTTGGCTTAATTCTCTCGGAA |
| | ACGATGAAATATACAAGTTATATCTTGGCTTTTCAGCTCTGCA |
| | TCGTTTTGGGTTCTCTTGGCTGTTACTGCCAGGACCCATATGT |
| | AAAAGAAGCAGAAAACCTTAAGAAATATTTTGTAAGTATGAC |
| | TTTTTAATAGTACTTGTTTGTGGTTGAAAATGACTGAATATCG |
| | ACTTGCTGTAGCATCTCTGATAGGCTGTCATCTCTTGTAGGCA |
| | GTCATTTTGAGATTTGGT |
| TNFalpha promoter: | GAGGAATGGGTTACAGGAGACCTCTGGGGAGATGTGACCAC |
| (SEQ ID NO: 09) | AGCAATGGGTAGGAGAATGTCCAGGGCTATGGAAGTCGAGT |
| | ATGGGGACCCCCCCTTAACGAAGACAGGGCCATGTAGAGGG |
| | CCCCAGGGAGTGAAAGAGCCTCCAGGACCTCCAGGTATGGA |
| | ATACAGGGGACGTTTAAGAAGATATGGCCACACACTGGGGC |
| | CCTGAGAAGTGAGAGCTTCATGAAAAAAATCAGGGACCCCA |
| | GAGTTCCTTGGAAGCCAAGACTGAAACCAGCATTATGAGTCT |
| | CCGGGTCAGAATGAAAGAAGAAGGCCTGCCCCAGTGGGGTC |
| | TGTGAATTCCCGGGGGTGATTTCACTCCCCGGGGCTGTCCCA |
| | GGCTTGTCCCTGCTACCCCCACCCAGCCTTTCCTGAGGCCTCA |
| | AGCCTGCCACCAAGCCCCCAGCTCCTTCTCCCCGCAGGGACC |
| | CAAACACAGGCCTCAGGACTCAACACAGCTTTTCCCTCCAAC |
| | CCCGTTTTCTCTCCCTCAAGGACTCAGCTTTCTGAAGCCCCTC |
| | CCAGTTCTAGTTCTATCTTTTTCCTGCATCCTGTCTGGAAGTT |
| | AGAAGGAAACAGACCACAGACCTGGTCCCCAAAAGAAATGG |
| | AGGCAATAGGTTTTGAGGGGCATGGGGACGGGGTTCAGCCTC |
| | CAGGGTCCTACACACAAATCAGTCAGTGGCCCAGAAGACCCC |
| | CCTCGGAATCGGAGCAGGGAGGATGGGGAGTGTGAGGGGTA |
| | TCCTTGATGCTTGTGTGTCCCCAACTTTCCAAATCCCCGCCCC |
| | CGCGATGGAGAAGAAACCGAGACAGAAGGTGCAGGGCCCAC |
| | TACCGCTTCCTCCAGATGAGCTCATGGGTTTCTCCACCAAGG |
| | AAGTTTTCCGCTGGTTGAATGATTCTTTCCCCGCCCTCCTCTC |
| | GCCCCAGGGACATATAAAGGCAGTTGTTGGCACACCCAGCC |
| | CAATGGGTTTAATGTTGTCCAATGAACATAATGTCCTCCAGCT |
| IFNalpha promoter: | CCATCCATGTTCTTGCAAATGACAGGATCTCATTCTTTTTTAT |
| (SEQ ID NO: 10) | GGCTAAGTAGTACTCCATTGTGTATAAGTGCCATATTTTCTTT |
| | ATCCATTCATCTGTTAGACACCTAAGTTGCTTCCAAATCTTAG |
| | CTATTGTGAATAGTGCTGCAATAAACATGGGAGTGTAAATAT |
| | TTTGTTGACATACTGATTTCATTTCCTTTGGATAAATACCCAG |
| | TAGTGGGATTGCTGGATCATATGGGGGAAAATGGAGATGGCT |
| | AACGGGCACAAAAATATAGTTAGAAAAAATGAATATGATTT |
| | AGTATTCGATAGCACAATAGGATGACTACTGTTAATGATAAT |
| | TTATTATATATTATAAAATAACTAAAATAGTATAAATGGGAT |
| | GTATGTAGCAGAGAGAAATGATAAATGTTTGAAGCATTGGAT |
| | ACTCCATTCACCCTGCTGTGATTATTATGAATTGTCTGCCTAT |
| | ATAAAAATATTTCACTTATTCCATAAACACAGACGCCTCTTAT |
| | GTACCCACAAAAATCTATTTTCAAAAAAGTTGCTCTAAGAAT |
| | ATAGTTATCAAGTTAAGTAAAATGTCAATAGCCTTTTAATTTA |
| | ATTTTTAATTGTTTTATCATTCTTTGCAATAATAAAACATTAA |
| | CTTTATACTTTTTAATTTAATGTATAGAATAGAGATATACATA |
| | GGATATGTAAATAGATACACAGTGTATATGTGATTAAAATAT |
| | AATGGGAGATTCAATCAGAAAAAAGTTTCTAAAAAGGCTCTG |
| | GGGTAAAAGAGGAAGGAAACAATAATGAAAAAAATGTGGTG |
| | AGAAAAACAGCTGAAAACCCATGTAAAGAGTGCATAAAGAA |
| | AGCAAAAAGAGAAGTAGAAAGTAACACAGGGGCATTTGGAA |
| | AATGTAAACGAGTATGTTCCCTATTTAAGGCTAGGCACAAAG |
| | CAAGGTCTTCAGAGAACCTGGAGCCTAAGGTTTAGGCTCACC |
| | CATTTC |
| IL-6 promoter: | AGTCTAGAGCCCATTTGCATGAGACCAAGGATCCTCCTGCAA |
| (SEQ ID NO: 11) | GAGACACCATCCTGAGGGAAGAGGGCTTCTGAACCAGCTTGA |
| | CCCAATAAGAAATTCTTGGGTGCCGACGCGGAAGCAGATTCA |
| | GAGCCTAGAGCCGTGCCTGCGTCCGTAGTTTCCTTCTAGCTTC |
| | TTTTGATTTCAAATCAAGACTTACAGGGAGAGGGAGCGATAA |
| | ACACAAACTCTGCAAGATGCCACAAGGTCCTCCTTTGACATC |
| | CCCAACAAAGAGGTGAGTAGTATTCTCCCCCTTTCTGCCCTG |
| | AACCAAGTGGGCTTCAGTAATTTCAGGGCTCCAGGAGACCTG |
| | GGGCCCATGCAGGTGCCCCAGTGAAACAGTGGTGAAGAGAC |
| | TCAGTGGCAATGGGGAGAGCACTGGCAGCACAAGGCAAACC |
| | TCTGGCACAGAGAGCAAAGTCCTCACTGGGAGGATTCCCAAG |
| | GGGTCACTTGGGAGAGGGCAGGGCAGCAGCCAACCTCCTCTA |
| | AGTGGGCTGAAGCAGGTGAAGAAAGTGGCAGAAGCCACGCG |
| | GTGGCAAAAAGGAGTCACACACTCCACCTGGAGACGCCTTGA |
| | AGTAACTGCACGAAATTTGAGGATGGCCAGGCAGTTCTACAA |
| | CAGCCGCTCACAGGGAGAGCCAGAACACAGAAGAACTCAGA |
| | TGACTGGTAGTATTACCTTCTTCATAATCCCAGGCTTGGGGGG |
| | CTGCGATGGAGTCAGAGGAAACTCAGTTCAGAACATCTTTGG |
| | TTTTTACAAATACAAATTAACTGGAACGCTAAATTCTAGCCT |
| | GTTAATCTGGTCACTGAAAAAAAATTTTTTTTTTTTCAAAAAA |
| | CATAGCTTTAGCTTATTTTTTTTCTCTTTGTAAAACTTCGTGCA |
| | TGACTTCAGCTTTACTCTTTGTCAAGACATGCCAAAGTGCTGA |
| | GTCACTAATAAAAGAAAAAAAGAAAGTAAAGGAAGAGTGGT |
| | TCTGCTTCTTAGCGCTAGCCTCAATGACGACCTAAGCTGCACT |
| | TTTCCCCCTAGTTGTGTCTTGCCATGCTAAAGGACGTCACATT |
| | GCACAATCTTAATAAGGTTTCCAATCAGCCCCACCCGCTCTG |
| | GCCCCACCCTCACCCTCCAACAAAGATTTATCAAATGTGGGA |
| | TTTTCCCATGAGTCTCAATATTAGAGTCTCAACCCCCAATAAA |
| | TATAGGACTGGAGATGTCTGAGGCTCATTCTGCC |
| IL-12 promoter: | CACTTTGATTTTCAGGGGTTCTGGACCCTGAACATGGGTTAA |
| GH05J159330 | ACCAGTGGTTCTCAAGGTGTGGTCTTAGCGCCAGCAGCATCT |
| (SEQ ID NO: 12) | GCTTCCCCTGGAAACTTTCTAGAAATGCATATTCTCAGGCCCT |
| | CATGCCTGCTGAATCAGACACTCTGGGGGTGGGACTCAGCCG |
| | TCTGTTGTAGCAGTGCTTCCAGGTTATCCTGACAGTCACTCAA |
| | ATTTTAGAACCACTAGGTTCTCTATATGGGAGAGAGTAGTCT |
| | TTGAACTTGGAAAACAAGAGAAGCTAAACCCCTACAGCAAG |
| | GGCTGGTGACCAGGTCGTTGCCAGAACCTGAAAGTTCGCCTC |
| | TGTATTACCGTTCCTGTCCCTAACCCAAGTCCTTCAGTTCTGG |
| | GTGCTCCAGCACACACTGCTTTGTGCTGCAGTGATACAAATG |
| | TATGGCTCATCTCCCCAGCTGGCGGGGAGGCATTTAACACAC |
| | TGACTTAATAAATATTTATTGAGTAAAAGTATTTGCTCCTAGG |
| | AAGCGGGATCCAGGTAAGCCCTTTTTTTCTCTCTCAACTGCTT |
| | CTAGCCCAGTGCTCTTTATGTAGTAAGCACTAAATAAACAAC |
| | TGCTAGATGTTGATCCAGAAAGTCACATTCCTTCTCTAAGCTT |
| | TAAGTTTCTCATCTTAAAAATAAGAGGATTGTATCAGATGGC |
| | TTGCCTTAGGTCTCTTTCAGCTCCAGAGCCCCAAATACCCTAT |
| | GGTTCTCTATTTAGAGATGTTCTTCCCCACAGACTGCCATAGA |
| | ACTCCTGTAATTTACTTAGTATTTGCTTGACAGTATGGAGAAG |
| | AAAGGGGAGAATCAAGATTTTATTTAAAAAAAAAGTAGCTA |
| | GAATGTGTATATGGTTCACAAAGGTAACAAGAATTATTGACA |
| | TTCTTTCTTCTCTTTTTTCTTCCTCTTCCTTCTCTTTTCCTCCTT |
| | CTCTTCCCCCTGCTTCTCTCCCTTCTTATAGATGTGTCACCAGC |
| | AGTTGGTCATCTCTTGGTTTT |

TABLE 2

| | Domain | |
|---|---|---|
| Receptor | (SEQ ID NO) | SEQUENCE |
| M-CSF R | Extracellular: aa | IPVIEPSVPELVVKPGATVTLRCVGNGSVEWDGPPSPHWT |
| | 20-517 | LYSDGSSSILSTNNATFQNTGTYRCTEPGDPLGGSAAIHL |
| | (SEQ ID NO: 13) | YVKDPARPWNVLAQEVVVFEDQDALLPCLLTDPVLEAG |
| | | VSLVRVRGRPLMRHTNYSFSPWHGFTIHRAKFIQSQDYQ |
| | | CSALMGGRKVMSISIRLKVQKVIPGPPALTLVPAELVRIR |
| | | GEAAQIVCSASSVDVNFDVFLQHNNTKLAIPQQSDFHNN |
| | | RYQKVLTLNLDQVDFQHAGNYSCVASNVQGKHSTSMFF |
| | | RVVESAYLNLSSEQNLIQEVTVGEGLNLKVMVEAYPGLQ |
| | | GFNWTYLGPFSDHQPEPKLANATTKDTYRHTFTLSLPRL |
| | | KPSEAGRYSFLARNPGGWRALTFELTLRYPPEVSVIWTFI |
| | | NGSGTLLCAASGYPQPNVTWLQCSGHTDRCDEAQVLQV |
| | | WDDPYPEVLSQEPFHKVTVQSLLTVETLEHNQTYECRAH |
| | | NSVGSGSWAFIPISAGAHTHPPDEFLFTP |
| | Transmembrane: | VVVACMSIMALLLLLLLLLLY |
| | aa 518-538 | |
| | (SEQ ID NO: 14) | |
| | Intracellular: aa | LQFGKTLGAGAFGKVVEATAFGLGKEDAVLKVAVKML |
| | 586-910 | KSTAHADEKEALMSELKIMSHLGQHENIVNLLGACTHGG |
| | (SEQ ID NO: 15) | PVLVITEYCCYGDLLNFLRRKAEAMLGPSLSPGQDPEGG |
| | | VDYKNIHLEKKYVRRDSGFSSQGVDTYVEMRPVSTSSND |
| | | SFSEQDLDKEDGRPLELRDLLHFSSQVAQGMAFLASKNCI |
| | | HRDVAARNVLLTNGHVAKIGDFGLARDIMNDSNYIVKG |
| | | NARLPVKWMAPESIFDCVYTVQSDVWSYGILLWEIFSLG |
| | | LNPYPGILVNSKFYKLVKDGYQMAQPAFAPKNIYSIMQA |
| | | CWALEPTHRPTFQQICSFL |
| CXCR4 | Total | MEGISIYTSDNYTEEMGSGDYDSMKEPCFREENANFNKIF |
| | (SEQ ID NO: 16) | LPTIYSIIFLTGIVGNGLVILVMGYQKKLRSMTDKYRLHLS |
| | | VADLLFVITLPFWAVDAVANWYFGNFLCKAVHVIYTVN |
| | | LYSSVLILAFISLDRYLAIVHATNSQRPRKLLAEKVVYVG |
| | | VWIPALLLTIPDFIFANVSEADDRYICDRFYPNDLWVVVF |
| | | QFQHIMVGLILPGIVILSCYCIIISKLSHSKGHQKRKALKTT |
| | | VILILAFFACWLPYYIGISIDSFILLEIIKQGCEFENTVHKWI |
| | | SITEALAFFHCCLNPILYAFLGAKFKTSAQHALTSVSRGSS |
| | | LKILSKGKRGGHSSVSTESESSSFHSS |
| Neuropilin | Extracellular: aa | RGQPDPPCGGRLNSKDAGYITSPGYPQDYPSHQNCEWIV |
| (NRP2): | 21-864 | YAPEPNQKIVLNFNPHFEIEKHDCKYDFIEIRDGDSESADL |
| Binds | (SEQ ID NO: 17) | LGKHCGNIAPPTIISSGSMLYIKFTSDYARQGAGFSLRYEI |
| SEMA3A | | FKTGSEDCSKNFTSPNGTIESPGFPEKYPHNLDCTFTILAK |
| | | PKMEIILQFLIFDLEHDPLQVGEGDCKYDWLDIWDGIPHV |
| | | GPLIGKYCGTKTPSELRSSTGILSLTFHTDMAVAKDGFSA |
| | | RYYLVHQEPLENFQCNVPLGMESGRIANEQISASSTYSDG |
| | | RWTPQQSRLHGDDNGWTPNLDSNKEYLQVDLRFLTMLT |
| | | AIATQGAISRETQNGYYVKSYKLEVSTNGEDWMVYRHG |
| | | KNHKVFQANNDATEVVLNKLHAPLLTRFVRIRPQTWHS |
| | | GIALRLELFGCRVTDAPCSNMLGMLSGLIADSQISASSTQ |
| | | EYLWSPSAARLVSSRSGWFPRIPQAQPGEEWLQVDLGTP |
| | | KTVKGVIIQGARGGDSITAVEARAFVRKFKVSYSLNGKD |
| | | WEYIQDPRTQQPKLFEGNMHYDTPDIRRFDPIPAQYVRV |
| | | YPERWSPAGIGMRLEVLGCDWTDSKPTVETLGPTVKSEE |
| | | TTTPYPTEEEATECGENCSFEDDKDLQLPSGFNCNFDFLE |
| | | EPCGWMYDHAKWLRTTWASSSSPNDRTFPDDRNFLRLQ |
| | | SDSQREGQYARLISPPVHLPRSPVCMEFQYQATGGRGVA |
| | | LQVVREASQESKLLWVIREDQGGEWKHGRIILPSYDMEY |
| | | QIVFEGVIGKGRSGEIAIDDIRISTDVPLENCMEPISAFAGE |
| | | NFKVDIPEIHEREGYEDEIDDEYEVDWSNSSSATSGSGAP |
| | | STDKEKSWLYTLDP |
| | Transmembrane: | ILITIIAMSSLGVLLGATCAGLLLY |
| | aa 865-889 | |
| | (SEQ ID NO: 18) | |
| | Cytoplasmic: aa | CTCSYSGLSSRSCTTLENYNFELYDGLKHKVKMNHQKCC |
| | 890-931 | SEA |
| | (SEQ ID NO: 19) | |
| Epidermal | Extracellular: aa | LEEKKVCQGTSNKLTQLGTFEDHFLSLQRMFNNCEVVLG |
| Growth | 25-645 | NLEITYVQRNYDLSFLKTIQEVAGYVLIALNTVERIPLENL |
| Factor | (SEQ ID NO: 20) | QIIRGNMYYENSYALAVLSNYDANKTGLKELPMRNLQEI |
| receptor: | | LHGAVRFSNNPALCNVESIQWRDIVSSDFLSNMSMDFQN |
| EGFR: | | HLGSCQKCDPSCPNGSCWGAGEENCQKLTKIICAQQCSG |
| | | RCRGKSPSDCCHNQCAAGCTGPRESDCLVCRKFRDEATC |
| | | KDTCPPLMLYNPTTYQMDVNPEGKYSFGATCVKKCPRN |
| | | YVVTDHGSCVRACGADSYEMEEDGVRKCKKCEGPCRK |
| | | VCNGIGIGEFKDSLSINATNIKHFKNCTSISGDLHILPVAFR |
| | | GDSFTHTPPLDPQELDILKTVKEITGFLLIQAWPENRTDLH |
| | | AFENLEIIRGRTKQHGQFSLAVVSLNITSLGLRSLKEISDG |
| | | DVIISGNKNLCYANTINWKKLFGTSGQKTKIISNRGENSC |
| | | KATGQVCHALCSPEGCWGPEPRDCVSCRNVSRGRECVD |
| | | KCNLLEGEPREFVENSECIQCHPECLPQAMNITCTGRGPD |
| | | NCIQCAHYIDGPHCVKTCPAGVMGENNTLVWKYADAG |
| | | HVCHLCHPNCTYGCTGPGLEGCPTNGPKIPS |
| | Transmembrane: | IATGMVGALLLLLVVALGIGLFM |
| | aa 646-668 | |
| | (SEQ ID NO: 21) | |
| | Intracellular: | RRRHIVRKRTLRRLLQERELVEPLTPSGEAPNQALLRILKE |
| | (SEQ ID NO: 22) | TEFKKIKVLGSGAFGTVYKGLWIPEGEKVKIPVAIKELRE |
| | | ATSPKANKEILDEAYVMASVDNPHVCRLLGICLTSTVQLI |
| | | TQLMPFGCLLDYVREHKDNIGSQYLLNWCVQIAKGMNY |
| | | LEDRRLVHRDLAARNVLVKTPQHVKITDFGLAKLLGAEE |
| | | KEYHAEGGKVPIKWMALESILHRIYTHQSDVWSYGVTV |
| | | WELMTFGSKPYDGIPASEISSILEKGERLPQPPICTIDVYMI |
| | | MVKCWMIDADSRPKFRELIIEFSKMARDPQRYLVIQGDE |
| | | RMHLPSPTDSNFYRALMDEEDMDDVVDADEYLIPQQGF |
| | | FSSPSTSRTPLLSSLSATSNNSTVACIDRNGLQSCPIKEDSF |
| | | LQRYSSDPTGALTEDSIDDTFLPVPEYINQSVPKRPAGSVQ |
| | | NPVYHNQPLNPAPSRDPHYQDPHSTAVGNPEYLNTVQPT |
| | | CVNSTFDSPAHWAQKGSHQISLDNPDYQQDFFPKEAKPN |
| | | GIFKGSTAENAEYLRVAPQSSEFIGA |
| Vascular | Extracellular: aa | ASVGLPSVSLDLPRLSIQKDILTIKANTTLQITCRGQRDLD |
| Endothelial | 20-764 | WLWPNNQSGSEQRVEVTECSDGLFCKTLTIPKVIGNDTG |
| Growth | (SEQ ID NO: 23) | AYKCFYRETDLASVIYVYVQDYRSPFIASVSDQHGVVYIT |
| Factor | | ENKNKTVVIPCLGSISNLNVSLCARYPEKRFVPDGNRISW |
| Receptor | | DSKKGFTIPSYMISYAGMVFCEAKINDESYQSIMYIVVVV |
| 2 | | GYRIYDVVLSPSHGIELSVGEKLVLNCTARTELNVGIDFN |
| | | WEYPSSKHQHKKLVNRDLKTQSGSEMKKFLSTLTIDGVT |
| | | RSDQGLYTCAASSGLMTKKNSTFVRVHEKPFVAFGSGM |
| | | ESLVEATVGERVRIPAKYLGYPPPEIKWYKNGIPLESNHTI |
| | | KAGHVLTIMEVSERDTGNYTVILTNPISKEKQSHVVSLVV |
| | | YVPPQIGEKSLISPVDSYQYGTTQTLTCTVYAIPPPHHIHW |
| | | YWQLEEECANEPSQAVSVTNPYPCEEWRSVEDFQGGNKI |
| | | EVNKNQFALIEGKNKTVSTLVIQAANVSALYKCEAVNKV |
| | | GRGERVISFHVTRGPEITLQPDMQPTEQESVSLWCTADRS |
| | | TFENLTWYKLGPQPLPIHVGELPTPVCKNLDTLWKLNAT |
| | | MFSNSTNDILIMELKNASLQDQGDYVCLAQDRKTKKRH |
| | | CVVRQLTVLERVAPTITGNLENQTTSIGESIEVSCTASGNP |
| | | PPQIMWFKDNETLVEDSGIVLKDGNRNLTIRRVRKEDEG |
| | | LYTCQACSVLGCAKVEAFFIIEGAQEKTNLE |
| | Transmembrane. | IIILVGTAVIAMFFWLLLVII |
| | aa 765-785 | |
| | (SEQ ID NO: 24) | |
| | Intracellular: aa | LRTVKRANGGELKTGYLSIVMDPDELPLDEHCERLPYDA |
| | 786-1356: | SKWEFPRDRLKLGKPLGRGAFGQVIEADAFGIDKTATCR |
| | (SEQ ID NO: 25) | TVAVKMLKEGATHSEHRALMSELKILIHIGHHLNVVNLL |
| | | GACTKPGGPLMVIVEFCKFGNLSTYLRSKRNEFVPYKTK |
| | | GARFRQGKDYVGAIPVDLKRRLDSITSSQSSASSGFVEEK |
| | | SLSDVEEEEAPEDLYKDFLTLEHLICYSFQVAKGMEFLAS |
| | | RKCIHRDLAARNILLSEKNVVKICDFGLARDIYKDPDYVR |
| | | KGDARLPLKWMAPETIFDRVYTIQSDVWSFGVLLWEIFS |
| | | LGASPYPGVKIDEEFCRRLKEGTRMRAPDYTTPEMYQTM |
| | | LDCWHGEPSQRPTFSELVEHLGNLLQANAQQDGKDYIVL |
| | | PISETLSMEEDSGLSLPTSPVSCMEEEEVCDPKFHYDNTA |
| | | GISQYLQNSKRKSRPVSVKTFEDIPLEEPEVKVIPDDNQTD |
| | | SGMVLASEELKTLEDRTKLSPSFGGMVPSKSRESVASEGS |
| | | NQTSGYQSGYHSDDTDTTVYSSEEAELLKLIEIGVQTGST |
| | | AQILQPDSGTTLSSPPV |
| Transforming | Extracellular: aa | TIPPHVQKSVNNDMIVTDNNGAVKFPQLCKFCDVRFSTC |
| Growth | 23-166 | DNQKSCMSNCSITSICEKPQEVCVAVWRKNDENITLETV |
| Factor | (SEQ ID NO: 26) | CHDPKLPYHDFILEDAASPKCIMKEKKKPGETFFMCSCSS |
| beta | | DECNDNIIFSEEYNTSNPDLLLVIFQ |
| Receptor | Transmembrane | VTGISLLPPLGVAISVIIIFY |
| 2: P37173 | aa 167-187 | |
| | (SEQ ID NO: 27) | |
| | Intracellular: aa | CYRVNRQQKLSSTWETGKTRKLMEFSEHCAIILEDDRSDI |
| | 188-567 | SSTCANNINHNTELLPIELDTLVGKGRFAEVYKAKLKQNT |
| | (SEQ ID NO: 28) | SEQFETVAVKIFPYEEYASWKTEKDIFSDINLKHENILQFL |
| | | TAEERKTELGKQYWLITAFHAKGNLQEYLTRHVISWEDL |
| | | RKLGSSLARGIAHLHSDHTPCGRPKNIPIVHRDLKSSNILV |
| | | KNDLTCCLCDFGLSLRLDPTLSVDDLANSGQVGTARYM |
| | | APEVLESRMNLENVESFKQTDVYSMALVLWEMTSRCNA |
| | | VGEVKDYEPPFGSKVREHPCVESMKDNVLRDRGRPEIPS |
| | | FWLNHQGIQMVCETLTECWDHDPEARLTAQCVAERFSE |
| | | LEHLDRLSGRSCSEEKIPEDGSLNTTK |
| Tumor | Extracellular: aa | LVPHLGDREKRDSVCPQGKYIHPQNNSICCTKCHKGTYL |
| necrosis | 30-211: | YNDCPGPGQDTDCRECESGSFTASENHLRHCLSCSKCRK |
| factor | (SEQ ID NO: 29) | EMGQVEISSCTVDRDTVCGCRKNQYRHYWSENLFQCFN |
| alpha | | CSLCLNGTVHLSCQEKQNTVCTCHAGFFLRENECVSCSN |
| receptor | | CKKSLECTKLCLPQIENVKGTEDSGTT |
| (P19348) | Transmembrane: | VLLPLVIFFGLCLLSLLFIGL |
| | aa 212-232 | |
| | (SEQ ID NO: 30) | |
| | Intracellular: aa | MYRYQRWKSKLYSIVCGKSTPEKEGELEGTTTKPLAPNP |
| | 233-455 | SFSPTPGFTPTLGFSPVPSSTFTSSSTYTPGDCPNFAAPRRE |
| | (SEQ ID NO: 31) | VAPPYQGADPILATALASDPIPNPLQKWEDSAHKPQSLDT |
| | | DDPATLYAVVENVPPLRWKEFVRRLGLSDHEIDRLELQN |
| | | GRCLREAQYSMLATWRRRTPRREATLELLGRVLRDMDL |
| | | LGCLEDIEEALCGPAALPPAPSLLR |
| Interleukin | Extracellular: aa | LAPRRCPAQEVARGVLTSLPGDSVTLTCPGVEPEDNATV |
| 6 | 20-365: | HWVLRKPAAGSHPSRWAGMGRRLLLRSVQLHDSGNYSC |
| receptor | (SEQ ID NO: 32) | YRAGRPAGTVHLLVDVPPEEPQLSCFRKSPLSNVVCEWG |
| (P08887): | | PRSTPSLTTKAVLLVRKFQNSPAEDFQEPCQYSQESQKFS |
| | | CQLAVPEGDSSFYIVSMCVASSVGSKFSKTQTFQGCGILQ |
| | | PDPPANITVTAVARNPRWLSVTWQDPHSWNSSFYRLRFE |
| | | LRYRAERSKTFTTWMVKDLQHHCVIHDAWSGLRHVVQL |
| | | RAQEEFGQGEWSEWSPEAMGTPWTESRSPPAENEVSTPM |
| | | QALTTNKDDDNILFRDSANATSLPVQDSSSVPLP |
| | Transmembrane: | TFLVAGGSLAFGTLLCIAIVL |
| | aa 366-386 | |
| | (SEQ ID NO: 33) | |
| | Intracellular: aa | RFKKTWKLRALKEGKTSMHPPYSLGQLVPERPRPTPVLV |
| | 387-468 | PLISPPVSPSSLGSDNTSSHNRPDARDPRSPYDISNTDYFFP |
| | (SEQ ID NO: 34) | R |
| Interferon | Extracellular: aa | EMGTADLGPSSVPTPTNVTIESYNMNPIVYWEYQIMPQV |
| gamma | 18-245 | PVFTVEVKNYGVKNSEWIDACINISHHYCNISDHVGDPSN |
| receptor 2 | (SEQ ID NO: 35) | SLWVRVKARVGQKESAYAKSEEFAVCRDGKIGPPKLDIR |
| (P15260): | | KEEKQIMIDIFHPSVFVNGDEQEVDYDPETTCYIRVYNVY |
| | | VRMNGSEIQYKILTQKEDDCDEIQCQLAIPVSSLNSQYCV |
| | | SAEGVLHVWGVTTEKSKEVCITIFNSSIKG |
| | Transmembrane: | SLWIPVVAALLLFLVLSLVFI |
| | aa 246-266 | |
| | (SEQ ID NO: 36) | |
| | Cytoplasmic: aa | CFYIKKINPLKEKSIILPKSLISVVRSATLETKPESKYVSLIT |
| | 267-489 | SYQPFSLEKEVVCEEPLSPATVPGMHTEDNPGKVEHTEEL |
| | (SEQ ID NO: 37) | SSITEVVTTEENIPDVVPGSHLTPIERESSSPLSSNQSEPGSI |
| | | ALNSYHSRNCSESDHSRNGFDTDSSCLESHSSLSDSEFPPN |
| | | NKGEIKTEGQELITVIKAPTSFGYDKPHVLVDLLVDDSGK |
| | | ESLIGYRPTEDSKEFS |
| Granulocyte- | Extracellular: aa | EKSDLRTVAPASSLNVRFDSRTMNLSWDCQENTTFSKCF |
| macrophages | 23-320 | LTDKKNRVVEPRLSNNECSCTFREICLHEGVTFEVHVNTS |
| colony- | (SEQ ID NO: 38) | QRGFQQKLLYPNSGREGTAAQNFSCFIYNADLMNCTWA |
| stimulating | | RGPTAPRDVQYFLYIRNSKRRREIRCPYYIQDSGTHVGCH |
| factor | | LDNLSGLTSRNYFLVNGTSREIGIQFFDSLLDTKKIERFNP |
| receptor | | PSNVTVRCNTTHCLVRWKQPRTYQKLSYLDFQYQLDVH |
| subunit | | RKNTQPGTENLLINVSGDLENRYNFPSSEPRAKHSVKIRA |
| alpha | | ADVRILNWSSWSEAIEFGSDDG |
| (P15509) | Transmembrane: | NLGSVYIYVLLIVGTLVCGIVLGFLF |
| | aa 321-346 | |
| | (SEQ ID NO: 39) | |
| | Cytoplasmic: aa | KRFLRIQRLFPPVPQIKDKLNDNHEVEDEIIWEEFTPEEGK |
| | 347-400 | GYREEVLTVKEIT |
| | (SEQ ID NO: 40) | |
| Toll Like | Extracellular aa | ESWEPCVEVVPNITYQCMELNFYKIPDNLPFSTKNLDLSF |
| Receptor | 24-631 | NPLRHLGSYSFFSFPELQVLDLSRCEIQTIEDGAYQSLSHL |
| 4: | (SEQ ID NO: 41) | STLILTGNPIQSLALGAFSGLSSLQKLVAVETNLASLENFPI |
| | | GHLKTLKELNVAHNLIQSFKLPEYFSNLTNLEHLDLSSNK |
| | | IQSIYCTDLRVLHQMPLLNLSLDLSLNPMNFIQPGAFKEIR |
| | | LHKLTLRNNFDSLNVMKTCIQGLAGLEVHRLVLGEFRNE |
| | | GNLEKFDKSALEGLCNLTIEEFRLAYLDYYLDDIIDLFNC |
| | | LTNVSSFSLVSVTIERVKDFSYNFGWQHLELVNCKFGQFP |
| | | TLKLKSLKRLTFTSNKGGNAFSEVDLPSLEFLDLSRNGLS |
| | | FKGCCSQSDFGTTSLKYLDLSFNGVITMSSNFLGLEQLEH |
| | | LDFQHSNLKQMSEFSVFLSLRNLIYLDISHTHTRVAFNGIF |
| | | NGLSSLEVLKMAGNSFQENFLPDIFTELRNLTFLDLSQCQ |
| | | LEQLSPTAFNSLSSLQVLNMSHNNFFSLDTFPYKCLNSLQ |
| | | VLDYSLNHIMTSKKQELQHFPSSLAFLNLTQNDFACTCEH |
| | | QSFLQWIKDQRQLLVEVERMECATPSDKQGMPVLSLNIT |
| | | CQMNK |
| | Transmembrane: | TIIGVSVLSVLVVSVVAVLVY |
| | aa 632-652 | |
| | (SEQ ID NO: 42) | |
| | Cytoplasmic: aa | KFYFHLMLLAGCIKYGRGENIYDAFVIYSSQDEDWVRNE |
| | 653-839: | LVKNLEEGVPPFQLCLHYRDFIPGVAIAANIIHEGFHKSRK |
| | (SEQ ID NO: 43) | VIVVVSQHFIQSRWCIFEYEIAQTWQFLSSRAGIIFIVLQKV |
| | | EKTLLRQQVELYRLLSRNTYLEWEDSVLGRHIFWRRLRK |
| | | ALLDGKSWNPEGTVGTGCNWQEATSI |

TABLE 3

| Element | |
|---|---|
| (SEQ ID NO) | SEQUENCE |
| HRE | TGTCACGTCCTGCACGACTCTAGT |
| (SEQ ID NO: 44) | |
| HRE-MiniTK- | TCGAGATCCGGCCCCGCCCAGCGTCTTGTCATTGGCGAATTC |
| luciferase | GAACACGCAGATGCAGTCGGGGCGGCGCGGTCCGAGGTCCA |
| (SEQ ID NO: 45) | CTTCGCATATTAAGGTGACGCGTGTGGCCTCGAACACCGAGC |
| | GACCCTGCAGCGACCCGCTTAACAGCGTCAACAGCGTGCCGC |
| | AGATCTAAGTAAGCTTGGCATTCCGGTACTGTTGGTAAAATG |
| | GAAGACGCCAAAAACATAAAGAAAGGCCCGGCGCCATTCTA |
| | TCCTCTAGAGGATGGAACCGCTGGAGAGCAACTGCATAAGGC |
| | TATGAAGAGATACGCCCTGGTTCCTGGAACAATTGCTTTTAC |
| | AGATGCACATATCGAGGTGAACATCACGTACGCGGAATACTT |
| | CGAAATGTCCGTTCGGTTGGCAGAAGCTATGAAACGATATGG |
| | GCTGAATACAAATCACAGAATCGTCGTATGCAGTGAAAACTC |
| | TCTTCAATTCTTTATGCCGGTGTTGGGCGCGTTATTTATCGGA |
| | GTTGCAGTTGCGCCCGCGAACGACATTTATAATGAACGTGAA |
| | TTGCTCAACAGTATGAACATTTCGCAGCCTACCGTAGTGTTTG |
| | TTTCCAAAAAGGGGTTGCAAAAAATTTTGAACGTGCAAAAAA |
| | AATTACCAATAATCCAGAAAATTATTATCATGGATTCTAAAA |
| | CGGATTACCAGGGATTTCAGTCGATGTACACGTTCGTCACAT |
| | CTCATCTACCTCCCGGTTTTAATGAATACGATTTTGTACCAGA |
| | GTCCTTTGATCGTGACAAAACAATTGCACTGATAATGAATTC |
| | CTCTGGATCTACTGGGTTACCTAAGGGTGTGGCCCTTCCGCAT |
| | AGAACTGCCTGCGTCAGATTCTCGCATGCCAGAGATCCTATT |
| | TTTGGCAATCAAATCATTCCGGATACTGCGATTTTAAGTGTTG |
| | TTCCATTCCATCACGGTTTTGGAATGTTTACTACACTCGGATA |
| | TTTGATATGTGGATTTCGAGTCGTCTTAATGTATAGATTTGAA |
| | GAAGAGCTGTTTTTACGATCCCTTCAGGATTACAAAATTCAA |
| | AGTGCGTTGCTAGTACCAACCCTATTTTCATTCTTCGCCAAAA |
| | GCACTCTGATTGACAAATACGATTTATCTAATTTACACGAAA |
| | TTGCTTCTGGGGGCGCACCTCTTTCGAAAGAAGTCGGGGAAG |
| | CGGTTGCAAAACGCTTCCATCTTCCAGGGATACGACAAGGAT |
| | ATGGGCTCACTGAGACTACATCAGCTATTCTGATTACACCCG |
| | AGGGGGATGATAAACCGGGCGCGGTCGGTAAAGTTGTTCCAT |
| | TTTTTGAAGCGAAGGTTGTGGATCTGGATACCGGGAAAACGC |
| | TGGGCGTTAATCAGAGAGGCGAATTATGTGTCAGAGGACCTA |
| | TGATTATGTCCGGTTATGTAAACAATCCGGAAGCGACCAACG |
| | CCTTGATTGACAAGGATGGATGGCTACATTCTGGAGACATAG |
| | CTTACTGGGACGAAGACGAACACTTCTTCATAGTTGACCGCT |
| | TGAAGTCTTTAATTAAATACAAAGGATATCAGGTGGCCCCCG |
| | CTGAATTGGAATCGATATTGTTACAACACCCCAACATCTTCG |
| | ACGCGGGCGTGGCAGGTCTTCCCGACGATGACGCCGGTGAAC |
| | TTCCCGCCGCCGTTGTTGTTTTGGAGCACGGAAAGACGATGA |
| | CGGAAAAAGAGATCGTGGATTACGTCGCCAGTCAAGTAACA |
| | ACCGCGAAAAAGTTGCGCGGAGGAGTTGTGTTTGTGGACGAA |
| | GTACCGAAAGGTCTTACCGGAAAACTCGACGCAAGAAAAAT |
| | CAGAGAGATCCTCATAAAGGCCAAGAAGGGCGGAAAGTCCA |
| | AATTGTAAAATGTAACTGTATTCAGCGATGACGAAATTCTTA |
| | GCTATTGTAATACTGCGATGAGTGGCAGGGCGGGGCGTAATT |
| | TTTTTAAGGCAGTTATTGGTGCCCTTAAACGCCTGGTTGCTAC |
| | GCCTGAATAAGTGATAATAAGCGGATGAATGGCAGAAATTC |
| | GCCGGATCTTTGTGAAGGAACCTTACTTCTGTGGTGTGACAT |
| | AATTGGACAAACTACCTACAGAGATTTAAAGCTCTAAGGTAA |
| | ATATAAAATTTTTAAGTGTATAATGTGTTAAACTACTGATTCT |
| | AATTGTTTGTGTATTTTAGATTCCAACCTATGGAACTGATGAA |
| | TGGGAGCAGTGGTGGAATGCCTTTAATGAGGAAAACCTGTTT |
| | TGCTCAGAAGAAATGCCATCTAGTGATGATGAGGCTACTGCT |
| | GACTCTCAACATTCTACTCCTCCAAAAAAGAAGAGAAAGGTA |
| | GAAGACCCCAAGGACTTTCCTTCAGAATTGCTAAGTTTTTTGA |
| | GTCATGCTGTGTTTAGTAATAGAACTCTTGCTTGCTTTGCTAT |
| | TTACACCACAAAGGAAAAAGCTGCACTGCTATACAAGAAAA |
| | TTATGGAAAAATATTCTGTAACCTTTATAAGTAGGCATAACA |
| | GTTATAATCATAACATACTGTTTTTTCTTACTCCACACAGGCA |
| | TAGAGTGTCTGCTATTAATAACTATGCTCAAAAATTGTGTACC |
| | TTTAGCTTTTTAATTTGTAAAGGGGTTAATAAGGAATATTTGA |
| | TGTATAGTGCCTTGACTAGAGATCATAATCAGCCATACCACA |
| | TTTGTAGAGGTTTTACTTGCTTTAAAAAACCTCCCACACCTCC |
| | CCCTGAACCTGAAACATAAAATGAATGCAATTGTTGTTGTTA |
| | ACTTGTTTATTGCAGCTTATAATGGTTACAAATAAAGCAATA |
| | GCATCACAAATTTCACAAATAAAGCATTTTTTTCACTGCATTC |
| | TAGTTGTGGTTTGTCCAAACTCATCAATGTATCTTATCATGTC |
| | TGGATCCGTCGACCGATGCCCTTGAGAGCCTTCAACCCAGTC |
| | AGCTCCTTCCGGTGGGCGCGGGGCATGACTATCGTCGCCGCA |
| | CTTATGACTGTCTTCTTTATCATGCAACTCGTAGGACAGGTGC |
| | CGGCAGCGCTCTTCCGCTTCCTCGCTCACTGACTCGCTGCGCT |
| | CGGTCGTTCGGCTGCGGCGAGCGGTATCAGCTCACTCAAAGG |
| | CGGTAATACGGTTATCCACAGAATCAGGGGATAACGCAGGA |
| | AAGAACATGTGAGCAAAAGGCCAGCAAAAGGCCAGGAACCG |
| | TAAAAAGGCCGCGTTGCTGGCGTTTTTCCATAGGCTCCGCCC |
| | CCCTGACGAGCATCACAAAAATCGACGCTCAAGTCAGAGGTG |
| | GCGAAACCCGACAGGACTATAAAGATACCAGGCGTTTCCCCC |
| | TGGAAGCTCCCTCGTGCGCTCTCCTGTTCCGACCCTGCCGCTT |
| | ACCGGATACCTGTCCGCCTTTCTCCCTTCGGGAAGCGTGGCG |
| | CTTTCTCATAGCTCACGCTGTAGGTATCTCAGTTCGGTGTAGG |
| | TCGTTCGCTCCAAGCTGGGCTGTGTGCACGAACCCCCCGTTC |
| | AGCCCGACCGCTGCGCCTTATCCGGTAACTATCGTCTTGAGTC |
| | CAACCCGGTAAGACACGACTTATCGCCACTGGCAGCAGCCAC |
| | TGGTAACAGGATTAGCAGAGCGAGGTATGTAGGCGGTGCTAC |
| | AGAGTTCTTGAAGTGGTGGCCTAACTACGGCTACACTAGAAG |
| | AACAGTATTTGGTATCTGCGCTCTGCTGAAGCCAGTTACCTTC |
| | GGAAAAAGAGTTGGTAGCTCTTGATCCGGCAAACAAACCACC |
| | GCTGGTAGCGGTGGTTTTTTTGTTTGCAAGCAGCAGATTACGC |
| | GCAGAAAAAAAGGATCTCAAGAAGATCCTTTGATCTTTTCTA |
| | CGGGGTCTGACGCTCAGTGGAACGAAAACTCACGTTAAGGG |
| | ATTTTGGTCATGAGATTATCAAAAAGGATCTTCACCTAGATC |
| | CTTTTAAATTAAAAATGAAGTTTTAAATCAATCTAAAGTATAT |
| | ATGAGTAAACTTGGTCTGACAGTTACCAATGCTTAATCAGTG |
| | AGGCACCTATCTCAGCGATCTGTCTATTTCGTTCATCCATAGT |
| | TGCCTGACTCCCCGTCGTGTAGATAACTACGATACGGGAGGG |
| | CTTACCATCTGGCCCCAGTGCTGCAATGATACCGCGAGACCC |
| | ACGCTCACCGGCTCCAGATTTATCAGCAATAAACCAGCCAGC |
| | CGGAAGGGCCGAGCGCAGAAGTGGTCCTGCAACTTTATCCGC |
| | CTCCATCCAGTCTATTAATTGTTGCCGGGAAGCTAGAGTAAG |
| | TAGTTCGCCAGTTAATAGTTTGCGCAACGTTGTTGCCATTGCT |
| | ACAGGCATCGTGGTGTCACGCTCGTCGTTTGGTATGGCTTCAT |
| | TCAGCTCCGGTTCCCAACGATCAAGGCGAGTTACATGATCCC |
| | CCATGTTGTGCAAAAAAGCGGTTAGCTCCTTCGGTCCTCCGA |
| | TCGTTGTCAGAAGTAAGTTGGCCGCAGTGTTATCACTCATGG |
| | TTATGGCAGCACTGCATAATTCTCTTACTGTCATGCCATCCGT |
| | AAGATGCTTTTCTGTGACTGGTGAGTACTCAACCAAGTCATTC |
| | TGAGAATAGTGTATGCGGCGACCGAGTTGCTCTTGCCCGGCG |
| | TCAATACGGGATAATACCGCGCCACATAGCAGAACTTTAAAA |
| | GTGCTCATCATTGGAAAACGTTCTTCGGGGCGAAAACTCTCA |
| | AGGATCTTACCGCTGTTGAGATCCAGTTCGATGTAACCCACT |
| | CGTGCACCCAACTGATCTTCAGCATCTTTTACTTTCACCAGCG |
| | TTTCTGGGTGAGCAAAAACAGGAAGGCAAAATGCCGCAAAA |
| | AAGGGAATAAGGGCGACACGGAAATGTTGAATACTCATACT |
| | CTTCCTTTTTCAATATTATTGAAGCATTTATCAGGGTTATTGT |
| | CTCATGAGCGGATACATATTTGAATGTATTTAGAAAAATAAA |
| | CAAATAGGGGTTCCGCGCACATTTCCCCGAAAAGTGCCACCT |
| | GACGCGCCCTGTAGCGGCGCATTAAGCGCGGCGGGTGTGGTG |
| | GTTACGCGCAGCGTGACCGCTACACTTGCCAGCGCCCTAGCG |
| | CCCGCTCCTTTCGCTTTCTTCCCTTCCTTTCTCGCCACGTTCGC |
| | CGGCTTTCCCCGTCAAGCTCTAAATCGGGGGCTCCCTTTAGG |
| | GTTCCGATTTAGTGCTTTACGGCACCTCGACCCCAAAAAACTT |
| | GATTAGGGTGATGGTTCACGTAGTGGGCCATCGCCCTGATAG |
| | ACGGTTTTTCGCCCTTTGACGTTGGAGTCCACGTTCTTTAATA |
| | GTGGACTCTTGTTCCAAACTGGAACAACACTCAACCCTATCT |
| | CGGTCTATTCTTTTGATTTATAAGGGATTTTGCCGATTTCGGC |
| | CTATTGGTTAAAAAATGAGCTGATTTAACAAAAATTTAACGC |
| | GAATTTTAACAAAATATTAACGCTTACAATTTGCCATTCGCCA |
| | TTCAGGCTGCGCAACTGTTGGGAAGGGCGATCGGTGCGGGCC |
| | TCTTCGCTATTACGCCAGCCCAAGCTACCATGATAAGTAAGT |
| | AATATTAAGGTACGTGGAGGTTTTACTTGCTTTAAAAAACCT |
| | CCCACACCTCCCCCTGAACCTGAAACATAAAATGAATGCAAT |
| | TGTTGTTGTTAACTTGTTTATTGCAGCTTATAATGGTTACAAA |
| | TAAAGCAATAGCATCACAAATTTCACAAATAAAGCATTTTTT |
| | TCACTGCATTCTAGTTGTGGTTTGTCCAAACTCATCAATGTAT |
| | CTTATGGTACTGTAACTGAGCTAACATAACCCGGGAGGTACC |
| | GAGCTCTGTCACGTCCTGCACGACTCTAGTTGTCACGTCCTGC |
| | ACGACTCTAGTTGTCACGTCCTGCACGACGCTAGC |

TABLE 4

| Element | SEQUENCE |
|---|---|
| M-C SFR | ATCCCAGTGATAGAGCCCAGTGTCCCCGAGCTGGTCGTGAAG |
| extracellular domain | CCAGGAGCAACGGTGACCTTGCGATGTGTGGGCAATGGCAGC |
| (SEQ ID NO: 46) | GTGGAATGGGATGGCCCCCCATCACCTCACTGGACCCTGTAC |
| | TCTGATGGCTCCAGCAGCATCCTCAGCACCAACAACGCTACC |
| | TTCCAAAACACGGGGACCTATCGCTGCACTGAGCCTGGAGAC |
| | CCCCTGGGAGGCAGCGCCGCCATCCACCTCTATGTCAAAGAC |
| | CCTGCCCGGCCCTGGAACGTGCTAGCACAGGAGGTGGTCGTG |
| | TTCGAGGACCAGGACGCACTACTGCCCTGTCTGCTCACAGAC |
| | CCGGTGCTGGAAGCAGGCGTCTCGCTGGTGCGTGTGCGTGGC |
| | CGGCCCCTCATGCGCCACACCAACTACTCCTTCTCGCCCTGGC |
| | ATGGCTTCACCATCCACAGGGCCAAGTTCATTCAGAGCCAGG |
| | ACTATCAATGCAGTGCCCTGATGGGTGGCAGGAAGGTGATGT |
| | CCATCAGCATCCGGCTGAAAGTGCAGAAAGTCATCCCAGGGC |
| | CCCCAGCCTTGACACTGGTGCCTGCAGAGCTGGTGCGGATTC |
| | GAGGGGAGGCTGCCCAGATCGTGTGCTCAGCCAGCAGCGTTG |
| | ATGTTAACTTTGATGTCTTCCTCCAACACAACAACACTAAGCT |
| | CGCAATCCCTCAACAATCTGACTTTCATAATAACCGTTACCA |
| | AAAAGTCCTGACCCTCAACCTCGATCAAGTAGATTTCCAACA |
| | TGCCGGCAACTACTCCTGCGTGGCCAGCAACGTGCAGGGCAA |
| | GCACTCCACCTCCATGTTCTTCCGGGTGGTAGAGAGTGCCTA |
| | CTTGAACTTGAGCTCTGAGCAGAACCTCATCCAGGAGGTGAC |
| | CGTGGGGGAGGGGCTCAACCTCAAAGTCATGGTGGAGGCCT |
| | ACCCAGGCCTGCAAGGTTTTAACTGGACCTACCTGGGACCCT |
| | TTTCTGACCACCAGCCTGAGCCCAAGCTTGCTAATGCTACCA |
| | CCAAGGACACATACAGGCACACCTTCACCCTCTCTCTGCCCC |
| | GCCTGAAGCCCTCTGAGGCTGGCCGCTACTCCTTCCTGGCCA |
| | GAAACCCAGGAGGCTGGAGAGCTCTGACGTTTGAGCTCACCC |
| | TTCGATACCCCCCAGAGGTAAGCGTCATATGGACATTCATCA |
| | ACGGCTCTGGCACCCTTTTGTGTGCTGCCTCTGGGTACCCCCA |
| | GCCCAACGTGACATGGCTGCAGTGCAGTGGCCACACTGATAG |
| | GTGTGATGAGGCCCAAGTGCTGCAGGTCTGGGATGACCCATA |
| | CCCTGAGGTCCTGAGCCAGGAGCCCTTCCACAAGGTGACGGT |
| | GCAGAGCCTGCTGACTGTTGAGACCTTAGAGCACAACCAAAC |
| | CTACGAGTGCAGGGCCCACAACAGCGTGGGGAGTGGCTCCTG |
| | GGCCTTCATACCCATCTCTGCAGGAGCCCACACGCATCCCCC |
| | GGATGAGTTCCTCTTCACACCA |
| CD28 | GAGAGCAAGTACGGACCGCCCTGCCCCCCTTGCCCT |
| transmembrane | |
| domain | |
| (SEQ ID NO: 47) | |
| M-CSFR | GTGGTGGTGGCGTGCATGAGCATTATGGCGCTGCTGCTGCTG |
| transmembrane | CTGCTGCTGCTGCTGCTGTAT |
| domain | |
| (SEQ ID NO: 48) | |
| IgG4 hinge domain | ATGTTCTGGGTGCTGGTGGTGGTCGGAGGCGTGCTGGCCTGC |
| (SEQ ID NO: 49) | TACAGCCTGCTGGTCACCGTGGCCTTCATCATCTTTTGGGTG |
| TLR4 cytoplasmic | AAGTTCTATTTTCACCTGATGCTTCTTGCTGGCTGCATAAAGT |
| signaling domain | ATGGTAGAGGTGAAAACATCTATGATGCCTTTGTTATCTACTC |
| (SEQ ID NO: 50) | AAGCCAGGATGAGGACTGGGTAAGGAATGAGCTAGTAAAGA |
| | ATTTAGAAGAAGGGGTGCCTCCATTTCAGCTCTGCCTTCACTA |
| | CAGAGACTTTATTCCCGGTGTGGCCATTGCTGCCAACATCATC |
| | CATGAAGGTTTCCATAAAAGCCGAAAGGTGATTGTTGTGGTG |
| | TCCCAGCACTTCATCCAGAGCCGCTGGTGTATCTTTGAATATG |
| | AGATTGCTCAGACCTGGCAGTTTCTGAGCAGTCGTGCTGGTA |
| | TCATCTTCATTGTCCTGCAGAAGGTGGAGAAGACCCTGCTCA |
| | GGCAGCAGGTGGAGCTGTACCGCCTTCTCAGCAGGAACACTT |
| | ACCTGGAGTGGGAGGACAGTGTCCTGGGGCGGCACATCTTCT |
| | GGAGACGACTCAGAAAAGCCCTGCTGGATGGTAAATCATGG |
| | AATCCAGAAGGAACAGTGGGTACAGGATGCAATTGGCAGGA |
| | AGCAACATCTATC |
| T2A | GGCGGCGGAGAGGGCAGAGGAAGTCTTCTAACATGCGGTGA |
| (SEQ ID NO: 51) | CGTGGAGGAGAATCCCGGCCCT |
| CD19t marker | ATGCCACCTCCTCGCCTCCTCTTCTTCCTCCTCTTCCTCACCCC |
| (SEQ ID NO: 52) | CATGGAAGTCAGGCCCGAGGAACCTCTAGTGGTGAAGGTGG |
| | AAGAGGGAGATAACGCTGTGCTGCAGTGCCTCAAGGGGACC |
| | TCAGATGGCCCCACTCAGCAGCTGACCTGGTCTCGGGAGTCC |
| | CCGCTTAAACCCTTCTTAAAACTCAGCCTGGGGCTGCCAGGC |
| | CTGGGAATCCACATGAGGCCCCTGGCCATCTGGCTTTTCATCT |
| | TCAACGTCTCTCAACAGATGGGGGGCTTCTACCTGTGCCAGC |
| | CGGGGCCCCCCTCTGAGAAGGCCTGGCAGCCTGGCTGGACAG |
| | TCAATGTGGAGGGCAGCGGGGAGCTGTTCCGGTGGAATGTTT |
| | CGGACCTAGGTGGCCTGGGCTGTGGCCTGAAGAACAGGTCCT |
| | CAGAGGGCCCCAGCTCCCCTTCCGGGAAGCTCATGAGCCCCA |
| | AGCTGTATGTGTGGGCCAAAGACCGCCCTGAGATCTGGGAGG |
| | GAGAGCCTCCGTGTGTCCCACCGAGGGACAGCCTGAACCAGA |
| | GCCTCAGCCAGGACCTCACCATGGCCCCTGGCTCCACACTCT |
| | GGCTGTCCTGTGGGGTACCCCCTGACTCTGTGTCCAGGGGCC |
| | CCCTCTCCTGGACCCATGTGCACCCCAAGGGGCCTAAGTCAT |
| | TGCTGAGCCTAGAGCTGAAGGACGATCGCCCGGCCAGAGAT |
| | ATGTGGGTAATGGAGACGGGTCTGTTGTTGCCCCGGGCCACA |
| | GCTCAAGACGCTGGAAAGTATTATTGTCACCGTGGCAACCTG |
| | ACCATGTCATTCCACCTGGAGATCACTGCTCGGCCAGTACTA |
| | TGGCACTGGCTGCTGAGGACTGGTGGCTGGAAGGTCTCAGCT |
| | GTGACTTTGGCTTATCTGATCTTCTGCCTGTGTTCCCTTGTGG |
| | GCATTCTTCATCTTCAAAGAGCCCTGGTCCTGAGGAGGAAAA |
| | GATAA |
| epHIV7.2 vector | GTTAGACCAGATCTGAGCCTGGGAGCTCTCTGGCTAACTAGG |
| sequences with | GAACCCACTGCTTAAGCCTCAATAAAGCTTGCCTTGAGTGCT |
| MCSFRxTLR4 | TCAAGTAGTGTGTGCCCGTCTGTTGTGTGACTCTGGTAACTAG |
| chimeric receptor | AGATCCCTCAGACCCTTTTAGTCAGTGTGGAAAATCTCTAGC |
| including: M-CSFR | AGTGGCGCCCGAACAGGGACTTGAAAGCGAAAGGGAAACCA |
| extracellular domain, | GAGGAGCTCTCTCGACGCAGGACTCGGCTTGCTGAAGCGCGC |
| CD28 | ACGGCAAGAGGCGAGGGGCGGCGACTGGTGAGTACGCCAAA |
| transmembrane | AATTTTGACTAGCGGAGGCTAGAAGGAGAGAGATGGGTGCG |
| domain, IgG4 hinge | AGAGCGTCAGTATTAAGCGGGGGAGAATTAGATCGATGGGA |
| domain, TLR4 | AAAAATTCGGTTAAGGCCAGGGGGAAAGAAAAAATATAAAT |
| cytoplasmic signaling | TAAAACATATAGTATGGGCAAGCAGGGAGCTAGAACGATTC |
| domain, T2A | GCAGTTAATCCTGGCCTGTTAGAAACATCAGAAGGCTGTAGA |
| sequence, and CD19t | CAAATACTGGGACAGCTACAACCATCCCTTCAGACAGGATCA |
| marker | GAAGAACTTAGATCATTATATAATACAGTAGCAACCCTCTAT |
| (SEQ ID NO: 53) | TGTGTGCATCAAAGGATAGAGATAAAAGACACCAAGGAAGC |
| | TTTAGACAAGATAGAGGAAGAGCAAAACAAAAGTAAGAAAA |
| | AAGCACAGCAAGCAGCAGCTGACACAGGACACAGCAATCAG |
| | GTCAGCCAAAATTACCCTATAGTGCAGAACATCCAGGGGCAA |
| | ATGGTACATCAGGCCATATCACCTAGAACTTTAAATGCATGG |
| | GTAAAAGTAGTAGAAGAGAAGGCTTTCAGCCCAGAAGTGAT |
| | ACCCATGTTTTCAGCATTATCAGAAGGAGCCACCCCACAAGA |
| | TTTAAACACCATGCTAAACACAGTGGGGGGACATCAAGCAGC |
| | CATGCAAATGTTAAAAGAGACCATCAATGAGGAAGCTGCAG |
| | GCAAAGAGAAGAGTGGTGCAGAGAGAAAAAAGAGCAGTGG |
| | GAATAGGAGCTTTGTTCCTTGGGTTCTTGGGAGCAGCAGGAA |
| | GCACTATGGGCGCAGCGTCAATGACGCTGACGGTACAGGCCA |
| | GACAATTATTGTCTGGTATAGTGCAGCAGCAGAACAATTTGC |
| | TGAGGGCTATTGAGGCGCAACAGCATCTGTTGCAACTCACAG |
| | TCTGGGGCATCAAGCAGCTCCAGGCAAGAATCCTGGCTGTGG |
| | AAAGATACCTAAAGGATCAACAGCTCCTGGGGATTTGGGGTT |
| | GCTCTGGAAAACTCATTTGCACCACTGCTGTGCCTTGGATCTA |
| | CAAATGGCAGTATTCATCCACAATTTTAAAAGAAAAGGGGGG |
| | ATTGGGGGGTACAGTGCAGGGGAAAGAATAGTAGACATAAT |
| | AGCAACAGACATACAAACTAAAGAATTACAAAAACAAATTA |
| | CAAAAATTCAAAATTTTCGGGTTTATTACAGGGACAGCAGAG |
| | ATCCAGTTTGGGGATCAATTGCATGAAGAATCTGCTTAGGGT |
| | TAGGCGTTTTGCGCTGCTTCGCGAGGATCTGCGATCGCTCCG |
| | GTGCCCGTCAGTGGGCAGAGCGCACATCGCCCACAGTCCCCG |
| | AGAAGTTGGGGGGAGGGGTCGGCAATTGAACCGGTGCCTAG |
| | AGAAGGTGGCGCGGGGTAAACTGGGAAAGTGATGTCGTGTA |
| | CTGGCTCCGCCTTTTTCCCGAGGGTGGGGGAGAACCGTATAT |
| | AAGTGCAGTAGTCGCCGTGAACGTTCTTTTTCGCAACGGGTTT |
| | GCCGCCAGAACACAGCTGGGCTAGCCGCCACCATGCCACCTC |
| | CTCGCCTCCTCTTCTTCCTCCTCTTCCTCACCCCCATGGAAGTC |
| | AGGATCCCAGTGATAGAGCCCAGTGTCCCCGAGCTGGTCGTG |
| | AAGCCAGGAGCAACGGTGACCTTGCGATGTGTGGGCAATGG |
| | CAGCGTGGAATGGGATGGCCCCCCATCACCTCACTGGACCCT |
| | GTACTCTGATGGCTCCAGCAGCATCCTCAGCACCAACAACGC |
| | TACCTTCCAAAACACGGGGACCTATCGCTGCACTGAGCCTGG |
| | AGACCCCCTGGGAGGCAGCGCCGCCATCCACCTCTATGTCAA |
| | AGACCCTGCCCGGCCCTGGAACGTGCTAGCACAGGAGGTGGT |
| | CGTGTTCGAGGACCAGGACGCACTACTGCCCTGTCTGCTCAC |
| | AGACCCGGTGCTGGAAGCAGGCGTCTCGCTGGTGCGTGTGCG |
| | TGGCCGGCCCCTCATGCGCCACACCAACTACTCCTTCTCGCCC |
| | TGGCATGGCTTCACCATCCACAGGGCCAAGTTCATTCAGAGC |
| | CAGGACTATCAATGCAGTGCCCTGATGGGTGGCAGGAAGGTG |
| | ATGTCCATCAGCATCCGGCTGAAAGTGCAGAAAGTCATCCCA |
| | GGGCCCCCAGCCTTGACACTGGTGCCTGCAGAGCTGGTGCGG |
| | ATTCGAGGGGAGGCTGCCCAGATCGTGTGCTCAGCCAGCAGC |
| | GTTGATGTTAACTTTGATGTCTTCCTCCAACACAACAACACTA |
| | AGCTCGCAATCCCTCAACAATCTGACTTTCATAATAACCGTTA |
| | CCAAAAAGTCCTGACCCTCAACCTCGATCAAGTAGATTTCCA |
| | ACATGCCGGCAACTACTCCTGCGTGGCCAGCAACGTGCAGGG |
| | CAAGCACTCCACCTCCATGTTCTTCCGGGTGGTAGAGAGTGC |
| | CTACTTGAACTTGAGCTCTGAGCAGAACCTCATCCAGGAGGT |
| | GACCGTGGGGGAGGGGCTCAACCTCAAAGTCATGGTGGAGG |
| | CCTACCCAGGCCTGCAAGGTTTTAACTGGACCTACCTGGGAC |
| | CCTTTTCTGACCACCAGCCTGAGCCCAAGCTTGCTAATGCTAC |
| | CACCAAGGACACATACAGGCACACCTTCACCCTCTCTCTGCC |
| | CCGCCTGAAGCCCTCTGAGGCTGGCCGCTACTCCTTCCTGGCC |
| | AGAAACCCAGGAGGCTGGAGAGCTCTGACGTTTGAGCTCACC |
| | CTTCGATACCCCCCAGAGGTAAGCGTCATATGGACATTCATC |
| | AACGGCTCTGGCACCCTTTTGTGTGCTGCCTCTGGGTACCCCC |
| | AGCCCAACGTGACATGGCTGCAGTGCAGTGGCCACACTGATA |
| | GGTGTGATGAGGCCCAAGTGCTGCAGGTCTGGGATGACCCAT |
| | ACCCTGAGGTCCTGAGCCAGGAGCCCTTCCACAAGGTGACGG |
| | TGCAGAGCCTGCTGACTGTTGAGACCTTAGAGCACAACCAAA |
| | CCTACGAGTGCAGGGCCCACAACAGCGTGGGGAGTGGCTCCT |
| | GGGCCTTCATACCCATCTCTGCAGGAGCCCACACGCATCCCC |
| | CGGATGAGTTCCTCTTCACACCAGAGAGCAAGTACGGACCGC |
| | CCTGCCCCCCTTGCCCTATGTTCTGGGTGCTGGTGGTGGTCGG |
| | AGGCGTGCTGGCCTGCTACAGCCTGCTGGTCACCGTGGCCTT |
| | CATCATCTTTTGGGTGAAGTTCTATTTTCACCTGATGCTTCTT |
| | GCTGGCTGCATAAAGTATGGTAGAGGTGAAAACATCTATGAT |
| | GCCTTTGTTATCTACTCAAGCCAGGATGAGGACTGGGTAAGG |
| | AATGAGCTAGTAAAGAATTTAGAAGAAGGGGTGCCTCCATTT |
| | CAGCTCTGCCTTCACTACAGAGACTTTATTCCCGGTGTGGCCA |
| | TTGCTGCCAACATCATCCATGAAGGTTTCCATAAAAGCCGAA |
| | AGGTGATTGTTGTGGTGTCCCAGCACTTCATCCAGAGCCGCT |
| | GGTGTATCTTTGAATATGAGATTGCTCAGACCTGGCAGTTTCT |
| | GAGCAGTCGTGCTGGTATCATCTTCATTGTCCTGCAGAAGGT |
| | GGAGAAGACCCTGCTCAGGCAGCAGGTGGAGCTGTACCGCCT |
| | TCTCAGCAGGAACACTTACCTGGAGTGGGAGGACAGTGTCCT |
| | GGGGCGGCACATCTTCTGGAGACGACTCAGAAAAGCCCTGCT |
| | GGATGGTAAATCATGGAATCCAGAAGGAACAGTGGGTACAG |
| | GATGCAATTGGCAGGAAGCAACATCTATCGGCGGCGGAGAG |
| | GGCAGAGGAAGTCTTCTAACATGCGGTGACGTGGAGGAGAA |
| | TCCCGGCCCTATGCCACCTCCTCGCCTCCTCTTCTTCCTCCTCT |
| | TCCTCACCCCCATGGAAGTCAGGCCCGAGGAACCTCTAGTGG |
| | TGAAGGTGGAAGAGGGAGATAACGCTGTGCTGCAGTGCCTC |
| | AAGGGGACCTCAGATGGCCCCACTCAGCAGCTGACCTGGTCT |
| | CGGGAGTCCCCGCTTAAACCCTTCTTAAAACTCAGCCTGGGG |
| | CTGCCAGGCCTGGGAATCCACATGAGGCCCCTGGCCATCTGG |
| | CTTTTCATCTTCAACGTCTCTCAACAGATGGGGGGCTTCTACC |
| | TGTGCCAGCCGGGGCCCCCCTCTGAGAAGGCCTGGCAGCCTG |
| | GCTGGACAGTCAATGTGGAGGGCAGCGGGGAGCTGTTCCGGT |
| | GGAATGTTTCGGACCTAGGTGGCCTGGGCTGTGGCCTGAAGA |
| | ACAGGTCCTCAGAGGGCCCCAGCTCCCCTTCCGGGAAGCTCA |
| | TGAGCCCCAAGCTGTATGTGTGGGCCAAAGACCGCCCTGAGA |
| | TCTGGGAGGGAGAGCCTCCGTGTGTCCCACCGAGGGACAGCC |
| | TGAACCAGAGCCTCAGCCAGGACCTCACCATGGCCCCTGGCT |
| | CCACACTCTGGCTGTCCTGTGGGGTACCCCCTGACTCTGTGTC |
| | CAGGGGCCCCCTCTCCTGGACCCATGTGCACCCCAAGGGGCC |
| | TAAGTCATTGCTGAGCCTAGAGCTGAAGGACGATCGCCCGGC |
| | CAGAGATATGTGGGTAATGGAGACGGGTCTGTTGTTGCCCCG |
| | GGCCACAGCTCAAGACGCTGGAAAGTATTATTGTCACCGTGG |
| | CAACCTGACCATGTCATTCCACCTGGAGATCACTGCTCGGCC |
| | AGTACTATGGCACTGGCTGCTGAGGACTGGTGGCTGGAAGGT |
| | CTCAGCTGTGACTTTGGCTTATCTGATCTTCTGCCTGTGTTCC |
| | CTTGTGGGCATTCTTCATCTTCAAAGAGCCCTGGTCCTGAGGA |
| | GGAAAAGATAAGCGGCCGCTCTAGACCCGGGCTGCAGGAAT |
| | TCGATATCAAGCTTATCGATAATCAACCTCTGGATTACAAAA |
| | TTTGTGAAAGATTGACTGGTATTCTTAACTATGTTGCTCCTTT |
| | TACGCTATGTGGATACGCTGCTTTAATGCCTTTGTATCATGCT |
| | ATTGCTTCCCGTATGGCTTTCATTTTCTCCTCCTTGTATAAATC |
| | CTGGTTGCTGTCTCTTTATGAGGAGTTGTGGCCCGTTGTCAGG |
| | CAACGTGGCGTGGTGTGCACTGTGTTTGCTGACGCAACCCCC |
| | ACTGGTTGGGGCATTGCCACCACCTGTCAGCTCCTTTCCGGG |
| | ACTTTCGCTTTCCCCCTCCCTATTGCCACGGCGGAACTCATCG |
| | CCGCCTGCCTTGCCCGCTGCTGGACAGGGGCTCGGCTGTTGG |
| | GCACTGACAATTCCGTGGTGTTGTCGGGGAAATCATCGTCCT |
| | TTCCTTGGCTGCTCGCCTGTGTTGCCACCTGGATTCTGCGCGG |
| | GACGTCCTTCTGCTACGTCCCTTCGGCCCTCAATCCAGCGGAC |
| | CTTCCTTCCCGCGGCCTGCTGCCGGCTCTGCGGCCTCTTCCGC |
| | GTCTTCGCCTTCGCCCTCAGACGAGTCGGATCTCCCTTTGGGC |
| | CGCCTCCCCGCATCGATACCGTCGACTAGCCGTACCTTTAAG |
| | ACCAATGACTTACAAGGCAGCTGTAGATCTTAGCCACTTTTT |
| | AAAAGAAAAGGGGGGACTGGAAGGGCTAATTCACTCCCAAA |
| | GAAGACAAGATCTGCTTTTTGCCTGTACTGGGTCTCTCTGGTT |
| | AGACCAGATCTGAGCCTGGGAGCTCTCTGGCTAACTAGGGAA |
| | CCCACTGCTTAAGCCTCAATAAAGCTTGCCTTGAGTGCTTCAA |
| | GTAGTGTGTGCCCGTCTGTTGTGTGACTCTGGTAACTAGAGAT |
| | CCCTCAGACCCTTTTAGTCAGTGTGGAAAATCTCTAGCAGAA |
| | TTCGATATCAAGCTTATCGATACCGTCGACCTCGAGGGGGGG |
| | CCCGGTACCGAGCTCGGATCCACTAGTCCAGTGTGGTGGAAT |
| | TCTGCAGATATCCAGCACAGTGGCGGCCACTCAAGTCTGGAG |
| | GGCACGTTAAAACCCGCTGATCAGCCTCGACTGTGCCTTCTA |
| | GTTGCCAGCCATCTGTTGTTTGCCCCTCCCCCGTGCCTTCCTT |
| | GACCCTGGAAGGTGCCACTCCCACTGTCCTTTCCTAATAAAA |
| | TGAGGAAATTGCATCGCATTGTCTGAGTAGGTGTCATTCTATT |
| | CTGGGGGGTGGGGTGGGGCAGGACAGCAAGGGGGAGGATTG |
| | GGAAGACAATAGCAGGCATGCTGGGGATGCGGTGGGCTCTA |
| | TGGCTTCTACTGGGCGGTTTTATGGACAGCAAGCGAACCGGA |
| | ATTGCCAGCTGGGGCGCCCTCTGGTAAGGTTGGGAAGCCCTG |
| | CAAAGTAAACTGGATGGCTTTCTTGCCGCCAAGGATCTGATG |
| | GCGCAGGGGATCAAGCTCTGATCAAGAGACAGGATGAGGAT |
| | CGTTTCGCATGATTGAACAAGATGGATTGCACGCAGGTTCTC |
| | CGGCCGCTTGGGTGGAGAGGCTATTCGGCTATGACTGGGCAC |
| | AACAGACAATCGGCTGCTCTGATGCCGCCGTGTTCCGGCTGT |
| | CAGCGCAGGGGCGCCCGGTTCTTTTTGTCAAGACCGACCTGT |
| | CCGGTGCCCTGAATGAACTGCAAGACGAGGCAGCGCGGCTAT |
| | CGTGGCTGGCCACGACGGGCGTTCCTTGCGCAGCTGTGCTCG |
| | ACGTTGTCACTGAAGCGGGAAGGGACTGGCTGCTATTGGGCG |
| | AAGTGCCGGGGCAGGATCTCCTGTCATCTCACCTTGCTCCTGC |
| | CGAGAAAGTATCCATCATGGCTGATGCAATGCGGCGGCTGCA |
| | TACGCTTGATCCGGCTACCTGCCCATTCGACCACCAAGCGAA |
| | ACATCGCATCGAGCGAGCACGTACTCGGATGGAAGCCGGTCT |
| | TGTCGATCAGGATGATCTGGACGAAGAGCATCAGGGGCTCGC |
| | GCCAGCCGAACTGTTCGCCAGGCTCAAGGCGAGCATGCCCGA |
| | CGGCGAGGATCTCGTCGTGACCCATGGCGATGCCTGCTTGCC |
| | GAATATCATGGTGGAAAATGGCCGCTTTTCTGGATTCATCGA |
| | CTGTGGCCGGCTGGGTGTGGCAGACCGCTATCAGGACATAGC |
| | GTTGGCTACCCGTGATATTGCTGAAGAGCTTGGCGGCGAATG |
| | GGCTGACCGCTTCCTCGTGCTTTACGGTATCGCCGCTCCCGAT |
| | TCGCAGCGCATCGCCTTCTATCGCCTTCTTGACGAGTTCTTCT |
| | GAATTATTAACGCTTACAATTTCCTGATGCGGTATTTTCTCCT |
| | TACGCATCTGTGCGGTATTTCACACCGCATACAGGTGGCACT |
| | TTTCGGGGAAATGTGCGCGGAACCCCTATTTGTTTATTTTTCT |
| | AAATACATTCAAATATGTATCCGCTCATGACCAAAATCCCTT |
| | AACGTGAGTTTTCGTTCCACTGAGCGTCAGACCCCGTAGAAA |
| | AGATCAAAGGATCTTCTTGAGATCCTTTTTTTCTGCGCGTAAT |
| | CTGCTGCTTGCAAACAAAAAAACCACCGCTACCAGCGGTGGT |
| | TTGTTTGCCGGATCAAGAGCTACCAACTCTTTTTCCGAAGGTA |
| | ACTGGCTTCAGCAGAGCGCAGATACCAAATACTGTTCTTCTA |
| | GTGTAGCCGTAGTTAGGCCACCACTTCAAGAACTCTGTAGCA |
| | CCGCCTACATACCTCGCTCTGCTAATCCTGTTACCAGTGGCTG |
| | CTGCCAGTGGCGATAAGTCGTGTCTTACCGGGTTGGACTCAA |
| | GACGATAGTTACCGGATAAGGCGCAGCGGTCGGGCTGAACG |
| | GGGGGTTCGTGCACACAGCCCAGCTTGGAGCGAACGACCTAC |
| | ACCGAACTGAGATACCTACAGCGTGAGCTATGAGAAAGCGC |
| | CACGCTTCCCGAAGGGAGAAAGGCGGACAGGTATCCGGTAA |
| | GCGGCAGGGTCGGAACAGGAGAGCGCACGAGGGAGCTTCCA |
| | GGGGGAAACGCCTGGTATCTTTATAGTCCTGTCGGGTTTCGC |
| | CACCTCTGACTTGAGCGTCGATTTTTGTGATGCTCGTCAGGGG |
| | GGCGGAGCCTATGGAAAAACGCCAGCAACGCGGCCTTTTTAC |
| | GGTTCCTGGCCTTTTGCTGGCCTTTTGCTCACATGTTCTTTCCT |
| | GCGTTATCCCCTGATTCTGTGGATAACCGTATTACCGCCTTTG |
| | AGTGAGCTGATACCGCTCGCCGCAGCCGAACGACCGAGCGC |
| | AGCGAGTCAGTGAGCGAGGAAGCGGAAGAGCGCCCAATACG |
| | CAAACCGCCTCTCCCCGCGCGTTGGCCGATTCATTAATGCAG |
| | CTGGCACGACAGGTTTCCCGACTGGAAAGCGGGCAGTGAGC |
| | GCAACGCAATTAATGTGAGTTAGCTCACTCATTAGGCACCCC |
| | AGGCTTTACACTTTATGCTTCCGGCTCGTATGTTGTGTGGAAT |
| | TGTGAGCGGATAACAATTTCACACAGGAAACAGCTATGACCA |
| | TGATTACGCCAAGCTCGAAATTAACCCTCACTAAAGGGAACA |
| | AAAGCTGGAGCTCCACCGCGGTGGCGGCCTCGAGGTCGAGAT |
| | CCGGTCGACCAGCAACCATAGTCCCGCCCCTAACTCCGCCCA |
| | TCCCGCCCCTAACTCCGCCCAGTTCCGCCCATTCTCCGCCCCA |
| | TGGCTGACTAATTTTTTTTATTTATGCAGAGGCCGAGGCCGCC |
| | TCGGCCTCTGAGCTATTCCAGAAGTAGTGAGGAGGCTTTTTT |
| | GGAGGCCTAGGCTTTTGCAAAAAGCTTCGACGGTATCGATTG |
| | GCTCATGTCCAACATTACCGCCATGTTGACATTGATTATTGAC |
| | TAGTTATTAATAGTAATCAATTACGGGGTCATTAGTTCATAGC |
| | CCATATATGGAGTTCCGCGTTACATAACTTACGGTAAATGGC |
| | CCGCCTGGCTGACCGCCCAACGACCCCCGCCCATTGACGTCA |
| | ATAATGACGTATGTTCCCATAGTAACGCCAATAGGGACTTTC |
| | CATTGACGTCAATGGGTGGAGTATTTACGGTAAACTGCCCAC |
| | TTGGCAGTACATCAAGTGTATCATATGCCAAGTACGCCCCCT |
| | ATTGACGTCAATGACGGTAAATGGCCCGCCTGGCATTATGCC |
| | CAGTACATGACCTTATGGGACTTTCCTACTTGGCAGTACATCT |
| | ACGTATTAGTCATCGCTATTACCATGGTGATGCGGTTTTGGCA |
| | GTACATCAATGGGCGTGGATAGCGGTTTGACTCACGGGGATT |
| | TCCAAGTCTCCACCCCATTGACGTCAATGGGAGTTTGTTTTGG |
| | CACCAAAATCAACGGGACTTTCCAAAATGTCGTAACAACTCC |
| | GCCCCATTGACGCAAATGGGCGGTAGGCGTGTACGGAATTCG |
| | GAGTGGCGAGCCCTCAGATCCTGCATATAAGCAGCTGCTTTT |
| | TGCCTGTACTGGGTCTCTCTG |

1. A polynucleotide comprising:
a first nucleic acid comprising a regulatory element, wherein the regulatory element is capable of or is configured to induce transcription of a therapeutic payload in a cell in an in vivo microenvironment; and
a second nucleic acid encoding the payload, wherein the therapeutic payload is operably-linked to the first nucleic acid.

2-56. (canceled)


SEQUENCE LISTING (54 sequences)

SEQ ID NO: 1 - DNA, 996 bp, Artificial Sequence
  APOE Promoter GH19J044900
gaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccct

SEQ ID NO: 2 - DNA, 800 bp, Artificial Sequence
  C1QA Promoter GH01J022636
gtgagccaaagcacctagacttgctgtttttttaaatgttggcaaaagctagtgactaagtgttcaccatgtgccaagtgttttgtatgtgcaagtcattaaaatgttataataatcagatgatataggaccccgttcccagtgtacagataaaactgagggctcaggaggtcaagtaatttcccaaagacatgcaactaggaagcagcaagtctagaaacagattgctgtgcttccttctcaccaatgcactgttctttcttctttgtattttccaagcttttggcaatgaattgcatgtcactttcataatcatgaaaagtcctacaagggacagatttttttaaagattccttccgctctggtgttctgtgcttcgctggttctcagaacctcaccctgcacggcctccaccccacatcctcacaggcactggcctcactgcccccactcccacaccagctgttgctggggcaggacgcccaatgtcccagtcttgctgaagtctgcttgaaatgtccctggtgagcttctggccactggggaagttcagggggcaggtctgaagaaggggaagtaggaagggatgtgaaacttggccacagcctggagccactcctgctgggcagcccacagggtccctgggcggagggcaggagcatccagttggagttgacaacaggaggcaggtgaggccagagtcccagagggagggggctgcagagttctgggacccaggggagctggcccaggaggctgggcagctgaggcagggagggagggaaataactgtgcctgat

SEQ ID NO: 3 - DNA, 1060 bp, Artificial Sequence
  SPP1 Promoter GH04J087974
caaagctaagattttgcctttaggaaagttttctttcctaataaaatagtttatttgacaactattctttttattaggatcattcatatatttgctaagcaaagagtaaatttattttccttaagattcaatttgaatatactaagaatattaaagcaagttagataaattacccaatatatttgtcaatttgaaatttgatagacattagttgtttaattcaatgggcagttttgagctgcagtttatacacacatgcataacagagtcacctttcaattatccatgttaataggaaagtggttatagattttagtacacacattaaaatatggatactcttctcttttgataaatctcatttcaaataaaaaaaccagtctcataattatgtatctgtatctattacatcattgaatttagtaaataatgtttaatatgtataaggaaaaacaatgttattgacatgaagattatactcacatatttggcttgaaaatatctataaaaataatttctgttgcaaagtaagaaatgttcttcagaatgttattaatccctgtgttaaaagagaaattggaagatgctcactttagctcctaaaagccatggtatgtactgtgaatgcaaagattctgaaactaaataaaaagaaagatagtaaaagactaatgtgctataaaggctaagggaaaataaaaacccatatattaattttcccggccatcttaattttcagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatc

SEQ ID NO: 4 - DNA, 958 bp, Artificial Sequence
  RGS1 promoter GH01J192575
accttgagcagtttttcctctgatactgtgaccttggtatcagtatgtttacaaattatgtcatatttgtatacaattaaacatttatttatttatgattggttttaattaaactttattttttcctaatatgcaaaatatgcaaaaatcaaagtttgatatactagttatattcttaataaatatgaaaataatattaaaaaccatgcaccccttaaaatcatcctatgtattgccagtcgatgtctagcacacaccttggaaaatattaacatggaaatttcctgccatggaaataagggatgagaattggagtgggaaaatgtgaagatagcctcttataactttccctcctatcagatatgtgcaaaataattaaacaaaaattatttgtttactttgccctgcttcctgcagaaaagaagagaactcttaaaatattttgatcagatgaattttatagtcaaggactatccaaagcatggagtaagatacaactctatgtgatgaatttctatgtgagtttctatatgataaattaatacgccaaatgaaacagtttattgaaacatgcaacttttttaaaataaacaaacatcctccagaaccaaagactgatgctgttaatgctttagacatgtgacatatgtgtgtgtacctctgtatgtttaattttctttctttctaatcaagtgacctgttctcgtttggttaagtttcaaccacaagacaccactgtatgctttttttttttcaagatacacgtcacagcacaccaagaaaaggggaacttccagtgtctgtggtaacatcacttgataaacagactcctttaaacagcaagtgcctgtctgcattctactatataaagcagcagagacgttgactagcgcatatttgctaagagcaccatgcgcgcagcagccatctccactccaaagttagacaaaatgccagg

SEQ ID NO: 5 - DNA, 1091 bp, Artificial Sequence
  C3 Promoter
ggaggaagaccacctttactgctatacacatttgtaccttttagatgttgatcaatatgaatatattatacacacagacacacacacagacacacacacacacacaaacaatacaatttaatatcctaagaggatattgacattagacaggtacaaaagctctagaaatgaggactttcctcagtgatgacttttttcaccaccaaagtcactcaggcatcctgacaagggtaagtgaggggagcctccttggaaaataaactcacttggatagtgaactcctgcacatacctcaaagcccatctgaaatgtcccctcctacaggaagttttccctgaccctccaagaagcagagttctatttcactggggaaaacatttcttcttcttcttttttttccctgccctgcacatgagctagaaaacatttcatgaaactgggagtttctgtgctgggctctgtccctcccccattctacttcccctccctcagcatggaagcctctggaagtggggctctgactcccagcctacagagagattcctaggaagtgttcgactgataaacgcatggccaaaagtgaactggggatgaggtccaagacatctgcggtggggggttctccagaccttagtgttcttccactacaaagtgggtccaacagagaaaggtctgtgttcaccaggtggccctgaccctgggagagtccagggcagggtgcagctgcattcatgctgctggggaacatgccctcaggttactcaccccatggacatgttggccccagggactgaaaagcttaggaaatggtattgagaaatctggggcagccccaaaaggggagaggccatggggagaagggggggctgagtgggggaaaggcaggagccagataaaaagccagctccagcaggcgctgctcactcctccccatcctctccctctgtccctctgtccctctgaccctgcactgtcccagcaccatgggacccacctcaggtcccagcctgctgctcctgctactaacccacctccccctggctctggggagtcccatgtgagtggttatgactctacccacaaacagggct

SEQ ID NO: 6 - DNA, 1014 bp, Artificial Sequence
  HSPA1B Promoter GH06J031813
gccttaaggacggcctacatactaaggaaaatttttttctaactcctggttgcagctgaggggagcggctgagggcggggacaggggtgcggcggacccactgctcccattacccgaccagcgcctcccttcctccttggatgggtgcccctgtcttgctaagaactgcctgtttacacaactgctttccttgtgaaaatttaaaggctcctattcccagttgttctatccttgtaggttaaagattatgtcaaaaactatattgcattatctctttccttctccttcccattaagacggaaaaaacatccgggagagccggtccgtttctcaggcagactaggccattaggtgcctcggagaaaggacccaaggctgctccgtccttcacagacacagtccaatcagagtttcccaggcacatcgatgcaccgcctccttcgagaaacaaggtaactttcgggttctggttgtctccaaagtcatccgaccaatctcgcaccgcccagagcgggcccttcctgtcaattacctactgaagggcaggcggccagcatcgccatggagaccaacacccttcccaccaccactccccctttctctcagggcccctgtcccctccagtgaatcccagaagactctggagagttctgagcagagggcggcaccctgccctctgattggtccaaggaaggctggggggcaggacgggaggcgaaacccctggaatattcccgacctggcagcctcatcgagcttggtgattggctcagaaggggaaaggcgggtctccacgacgacttataaaagccgaggggcgcgcggtccggaaaacggccagcctgaggagctgctgcgagggtccgcttcgtctttcgagagtgactcccgcggtcccaaggctttccagagcgaacctgtgcggctgcaggcaccggcgtgttgagtttccggcgttccgaaggactgagctcttgtcgcggatcccgtccgccgtttccagcccccagtctc

SEQ ID NO: 7 - DNA, 933 bp, Artificial Sequence
  TREM2 promoter GH06J041163
tgatcaggagttcaagcatgtgtgtgcacaaaataaacaccagtgtgagcatgtgtgcacaggagacacccaacagttccaagaaggctaaacttgggcagaaaattccaggtgggagagaaaattttctgtcttatggacagcccatttcccttttcccttctaactaggataatggtaatagttagtatttgttgaatgctgtgtgtcaggccctactggaaagcactttacctgtaggaacccatatggtgctcctgataaccctttgcactatcattattcccactgtatagatcagggaacagacacaggtaggttttggatgtgtggttacacacccagaaagtcaggaagtctggctccagagctgtgtacttaactgctgccacactacaggaatgacagccctggggggatgaactaagaggtgctggatgagggtcctggcctctaaaggcacagctgttctccaactcttgcaaggctgaaaccagaagatggcgggcattgcagctggtggagggtctgaatacagctgtgaggatagtgatccctgggctaggctctgcaaggaaactgagcagtgcagggccttaccagccccaaccatctgggggccaccctggctggcaccagcaggagggtgggctggcttctcagaggtctgggagactcagcctccttctgccagggctgcagtggccgactcctcctcccctctgtccccaccctgcaccgcctccagaccccagtcctgactattgcttaatccccaggagcccagttcctgtgggcagcgcctgacatgcctgatcctctcttttctgcagttcaagggaaagacgagatcttgcacaaggcactctgcttctgcccttggctggggaagggtggcatggagcctctccggctgctcatcttactctttgt

SEQ ID NO: 8 - DNA, 907 bp, Artificial Sequence
  IFNgamma promoter
gcagtgctgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttgtaagtatgactttttaatagtacttgtttgtggttgaaaatgactgaatatcgacttgctgtagcatctctgataggctgtcatctcttgtaggcagtcattttgagatttggt

SEQ ID NO: 9 - DNA, 960 bp, Artificial Sequence
  TNFalpha promoter
gaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagcc

SEQ ID NO: 10 - DNA, 1020 bp, Artificial Sequence
  IFNalpha promoter
caatgggtttaatgttgtccaatgaacataatgtcctccagctccatccatgttcttgcaaatgacaggatctcattcttttttatggctaagtagtactccattgtgtataagtgccatattttctttatccattcatctgttagacacctaagttgcttccaaatcttagctattgtgaatagtgctgcaataaacatgggagtgtaaatattttgttgacatactgatttcatttcctttggataaatacccagtagtgggattgctggatcatatgggggaaaatggagatggctaacgggcacaaaaatatagttagaaaaaatgaatatgatttagtattcgatagcacaataggatgactactgttaatgataatttattatatattataaaataactaaaatagtataaatgggatgtatgtagcagagagaaatgataaatgtttgaagcattggatactccattcaccctgctgtgattattatgaattgtctgcctatataaaaatatttcacttattccataaacacagacgcctcttatgtacccacaaaaatctattttcaaaaaagttgctctaagaatatagttatcaagttaagtaaaatgtcaatagccttttaatttaatttttaattgttttatcattctttgcaataataaaacattaactttatactttttaatttaatgtatagaatagagatatacataggatatgtaaatagatacacagtgtatatgtgattaaaatataatgggagattcaatcagaaaaaagtttctaaaaaggctctggggtaaaagaggaaggaaacaataatgaaaaaaatgtggtgagaaaaacagctgaaaacccatgtaaagagtgcataaagaaagcaaaaagagaagtagaaagtaacacaggggcatttggaaaatgtaaacgagtatgttccctatttaaggctaggcacaaagcaaggtcttcagagaacctggagcctaaggtttaggctcacccatttc

SEQ ID NO: 11 - DNA, 1214 bp, Artificial Sequence
  IL-6 promoter
agtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggtgagtagtattctccccctttctgccctgaaccaagtgggcttcagtaatttcagggctccaggagacctggggcccatgcaggtgccccagtgaaacagtggtgaagagactcagtggcaatggggagagcactggcagcacaaggcaaacctctggcacagagagcaaagtcctcactgggaggattcccaaggggtcacttgggagagggcagggcagcagccaacctcctctaagtgggctgaagcaggtgaagaaagtggcagaagccacgcggtggcaaaaaggagtcacacactccacctggagacgccttgaagtaactgcacgaaatttgaggatggccaggcagttctacaacagccgctcacagggagagccagaacacagaagaactcagatgactggtagtattaccttcttcataatcccaggcttggggggctgcgatggagtcagaggaaactcagttcagaacatctttggtttttacaaatacaaattaactggaacgctaaattctagcctgttaatctggtcactgaaaaaaaattttttttttttcaaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgccatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgcc

SEQ ID NO: 12 - DNA, 999 bp, Artificial Sequence
  IL-12 promoter GH05J159330
cactttgattttcaggggttctggaccctgaacatgggttaaaccagtggttctcaaggtgtggtcttagcgccagcagcatctgcttcccctggaaactttctagaaatgcatattctcaggccctcatgcctgctgaatcagacactctgggggtgggactcagccgtctgttgtagcagtgcttccaggttatcctgacagtcactcaaattttagaaccactaggttctctatatgggagagagtagtctttgaacttggaaaacaagagaagctaaacccctacagcaagggctggtgaccaggtcgttgccagaacctgaaagttcgcctctgtattaccgttcctgtccctaacccaagtccttcagttctgggtgctccagcacacactgctttgtgctgcagtgatacaaatgtatggctcatctccccagctggcggggaggcatttaacacactgacttaataaatatttattgagtaaaagtatttgctcctaggaagcgggatccaggtaagccctttttttctctctcaactgcttctagcccagtgctctttatgtagtaagcactaaataaacaactgctagatgttgatccagaaagtcacattccttctctaagctttaagtttctcatcttaaaaataagaggattgtatcagatggcttgccttaggtctctttcagctccagagccccaaataccctatggttctctatttagagatgttcttccccacagactgccatagaactcctgtaatttacttagtatttgcttgacagtatggagaagaaaggggagaatcaagattttatttaaaaaaaaagtagctagaatgtgtatatggttcacaaaggtaacaagaattattgacattctttcttctcttttttcttcctcttccttctcttttcctccttctcttccccctgcttctctcccttcttatagatgtgtcaccagcagttggtcatctcttggtttt

SEQ ID NO: 13 - protein, 498 aa, Artificial Sequence
  M-CSF R, extracellular aa 20-517
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTP

SEQ ID NO: 14 - protein, 21 aa, Artificial Sequence
  M-CSF R, transmembrane aa 518-538
VVVACMSIMALLLLLLLLLLY

SEQ ID NO: 15 - protein, 329 aa, Artificial Sequence
  M-CSF R, intracellular aa 586-910
LQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFL

SEQ ID NO: 16 - protein, 352 aa, Artificial Sequence
  CXCR4
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 17 - protein, 844 aa, Artificial Sequence
  Neuropilin (NRP2) Binds SEMA3A, extracellular aa 21-864
RGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDP

SEQ ID NO: 18 - protein, 25 aa, Artificial Sequence
  Neuropilin (NRP2) Binds SEMA3A, transmembrane aa 865-889
ILITIIAMSSLGVLLGATCAGLLLY

SEQ ID NO: 19 - protein, 42 aa, Artificial Sequence
  Neuropilin (NRP2) Binds SEMA3A, cytoplasmic aa 890-931
CTCSYSGLSSRSCTTLENYNFELYDGLKHKVKMNHQKCCSEA

SEQ ID NO: 20 - protein, 621 aa, Artificial Sequence [sequence omitted]

SEQ ID NO: 21 - protein, 23 aa, Artificial Sequence
  Epidermal Growth Factor receptor EGFR transmembrane aa 646-668
IATGMVGALLLLLVVALGIGLFM

SEQ ID NO: 22 - protein, 542 aa, Artificial Sequence
  Epidermal Growth Factor receptor EGFR Intracellular
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 23 - protein, 745 aa, Artificial Sequence
  Vascular Endothelial Growth Factor Receptor 2, extracellular aa 20-764
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 24 - protein, 21 aa, Artificial Sequence
  Vascular Endothelial Growth Factor Receptor 2, transmembrane aa 765-785
IIILVGTAVIAMFFWLLLVII

SEQ ID NO: 25 - protein, 571 aa, Artificial Sequence
  Vascular Endothelial Growth Factor Receptor 2, intracellular aa 786-1356
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 26 - protein, 144 aa, Artificial Sequence
  Transforming Growth Factor beta Receptor 2 P37173, extracellular aa 23-166
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 27 - protein, 21 aa, Artificial Sequence
  Transforming Growth Factor beta Receptor 2 P37173, transmembrane aa 167-187
VTGISLLPPLGVAISVIIIFY

SEQ ID NO: 28 - protein, 380 aa, Artificial Sequence
  Transforming Growth Factor beta Receptor 2 P37173, intracellular  aa 188-567
CYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 29 - protein, 182 aa, Artificial Sequence
  Tumor necrosis factor alpha receptor (P19348) , extracellular aa 30-211
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 30 - protein, 21 aa, Artificial Sequence
  Tumor necrosis factor alpha receptor (P19348), transmembrane aa 212-232
VLLPLVIFFGLCLLSLLFIGL

SEQ ID NO: 31 - protein, 223 aa, Artificial Sequence
  Tumor necrosis factor alpha receptor (P19348), intracellular aa 233-455
MYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 32 - protein, 346 aa, Artificial Sequence
  Interleukin 6 receptor (P08887) extracellular aa 20-365
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLP

SEQ ID NO: 33 - protein, 21 aa, Artificial Sequence
  Interleukin 6 receptor (P08887) transmembrane aa 366-386
TFLVAGGSLAFGTLLCIAIVL

SEQ ID NO: 34 - protein, 82 aa, Artificial Sequence
  Interleukin 6 receptor (P08887) intracellular aa 387-468
RFKKTWKLRALKEGKTSMHPPYSLGQLVPERPRPTPVLVPLISPPVSPSSLGSDNTSSHNRPDARDPRSPYDISNTDYFFPR

SEQ ID NO: 35 - protein, 228 aa, Artificial Sequence [sequence omitted]

SEQ ID NO: 36 - protein, 21 aa, Artificial Sequence
  Interferon gamma receptor 2 (P15260) transmembrane aa 246-266
SLWIPVVAALLLFLVLSLVFI

SEQ ID NO: 37 - protein, 223 aa, Artificial Sequence
  Interferon gamma receptor 2 (P15260) cytoplasmic aa 267-489
CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 38 - protein, 298 aa, Artificial Sequence
  Granulocyte-macrophages colony-stimulating factor receptor subunit alpha (P15509), extracellular aa 23-320
EKSDLRTVAPASSLNVRFDSRTMNLSWDCQENTTFSKCFLTDKKNRVVEPRLSNNECSCTFREICLHEGVTFEVHVNTSQRGFQQKLLYPNSGREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQLDVHRKNTQPGTENLLINVSGDLENRYNFPSSEPRAKHSVKIRAADVRILNWSSWSEAIEFGSDDG

SEQ ID NO: 39 - protein, 26 aa, Artificial Sequence
  Granulocyte-macrophages colony-stimulating factor receptor subunit alpha (P15509), transmembrane aa 321-346
NLGSVYIYVLLIVGTLVCGIVLGFLF

SEQ ID NO: 40 - protein, 54 aa, Artificial Sequence
  Granulocyte-macrophages colony-stimulating factor receptor subunit alpha (P15509), cytoplasmic aa 347-400
KRFLRIQRLFPPVPQIKDKLNDNHEVEDEIIWEEFTPEEGKGYREEVLTVKEIT

SEQ ID NO: 41 - protein, 608 aa, Artificial Sequence
  Toll Like Receptor 4 extracellular aa 24-631
ESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNK

SEQ ID NO: 42 - protein, 21 aa, Artificial Sequence
  Toll Like Receptor 4  transmembrane aa 632-652
TIIGVSVLSVLVVSVVAVLVY

SEQ ID NO: 43 - protein, 187 aa, Artificial Sequence
  Toll Like Receptor 4  cytoplasmic aa 653-839
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 44 - DNA, 24 bp, Artificial Sequence
  HRE
tgtcacgtcctgcacgactctagt

SEQ ID NO: 45 - DNA, 5820 bp, Artificial Sequence
  HRE-MiniTK- luciferase
tcgagatccggccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatctaagtaagcttggcattccggtactgttggtaaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatactgcgatgagtggcagggcggggcgtaatttttttaaggcagttattggtgcccttaaacgcctggttgctacgcctgaataagtgataataagcggatgaatggcagaaattcgccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataacccgggaggtaccgagctctgtcacgtcctgcacgactctagttgtcacgtcctgcacgactctagttgtcacgtcctgcacgacgctagc

SEQ ID NO: 46 - DNA, 1494 bp, Artificial Sequence
  M-CSFR extracellular domain
atcccagtgatagagcccagtgtccccgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacactaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacacca

SEQ ID NO: 47 - DNA, 36 bp, Artificial Sequence
  CD28 transmembrane domain
gagagcaagtacggaccgccctgccccccttgccct

SEQ ID NO: 48 - DNA, 63 bp, Artificial Sequence
  M-CSFR transmembrane domain
gtggtggtggcgtgcatgagcattatggcgctgctgctgctgctgctgctgctgctgctgtat

SEQ ID NO: 49 - DNA, 84 bp, Artificial Sequence
  IgG4 hinge domain
atgttctgggtgctggtggtggtcggaggcgtgctggcctgctacagcctgctggtcaccgtggccttcatcatcttttgggtg

SEQ ID NO: 50 - DNA, 561 bp, Artificial Sequence
  TLR4 cytoplasmic signaling domain
aagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatc

SEQ ID NO: 51 - DNA, 63 bp, Artificial Sequence
  T2A
ggcggcggagagggcagaggaagtcttctaacatgcggtgacgtggaggagaatcccggccct

SEQ ID NO: 52 - DNA, 972 bp, Artificial Sequence
  CD19t marker
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagataa

SEQ ID NO: 53 - DNA, 9534 bp, Artificial Sequence
  epHIV7.2 vector sequences with MCSFRxTLR4 chimeric receptor including M-CSFR extracellular domain, CD28 transmembrane domain, IgG4 hinge domain, TLR4 cytoplasmic signaling domain, T2A sequence, and CD19t marker
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgccacctcctcgcctcctcttcttcctcctcttcctcacccccatggaagtcaggatcccagtgatagagcccagtgtccccgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacactaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagagagcaagtacggaccgccctgccccccttgccctatgttctgggtgctggtggtggtcggaggcgtgctggcctgctacagcctgctggtcaccgtggccttcatcatcttttgggtgaagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatcggcggcggagagggcagaggaagtcttctaacatgcggtgacgtggaggagaatcccggccctatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagataagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 54 - DNA, 26 bp, Artificial Sequence
  HRE from an EPO gene
ccgggtagctggcgtacgtgctgcag